SEQUENCE LISTING (47 sequences)

SEQ ID NO: 1 - protein, 10 aa, synthetic construct
LALGETTRPA

SEQ ID NO: 2 - protein, 10 aa, synthetic construct
LANETITRPA

SEQ ID NO: 3 - protein, 10 aa, synthetic construct
LAKAGQANNA

SEQ ID NO: 4 - protein, 10 aa, synthetic construct
LAKDPKTTNA

SEQ ID NO: 5 - protein, 7 aa, synthetic construct
KDTDTTR

SEQ ID NO: 6 - protein, 7 aa, synthetic construct
RAGGSVG

SEQ ID NO: 7 - protein, 7 aa, synthetic construct
AVDTTKF

SEQ ID NO: 8 - protein, 7 aa, synthetic construct
STGKVPN

SEQ ID NO: 9 - protein, 10 aa, synthetic construct
LAKDTDTTRA

SEQ ID NO: 10 - protein, 10 aa, synthetic construct
LARAGGSVGA

SEQ ID NO: 11 - protein, 10 aa, synthetic construct
LAAVDTTKFA

SEQ ID NO: 12 - protein, 10 aa, synthetic construct
LASTGKVPNA

SEQ ID NO: 13 - protein, 735 aa, Adeno-associated dependoparvovirus A [sequence omitted]

SEQ ID NO: 14 - protein, 7 aa, synthetic construct
LGETTRP

SEQ ID NO: 15 - protein, 7 aa, synthetic construct
NETITRP

SEQ ID NO: 16 - protein, 7 aa, synthetic construct
KAGQANN

SEQ ID NO: 17 - protein, 7 aa, synthetic construct
KDPKTTN

SEQ ID NO: 18 - protein, 7 aa, synthetic construct
KDTDTTR

SEQ ID NO: 19 - protein, 7 aa, synthetic construct
RAGGSVG

SEQ ID NO: 20 - protein, 7 aa, synthetic construct
AVDTTKF

SEQ ID NO: 21 - protein, 7 aa, synthetic construct
STGKVPN

SEQ ID NO: 22 - DNA, 293 bp, synthetic construct
  source 1..293: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattacca

SEQ ID NO: 23 - DNA, 220 bp, synthetic construct
  source 1..220: mol_type = other DNA
tgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 24 - DNA, 318 bp, synthetic construct
  source 1..318: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcga

SEQ ID NO: 25 - DNA, 102 bp, synthetic construct
  source 1..102: mol_type = other DNA
ccagctgttggggtgagtactccctctcaaaagcgggcattacttctgcgctaagattgtcagtttccaaaaacgaggaggatttgatattcacctggcccg

SEQ ID NO: 26 - DNA, 810 bp, synthetic construct
  source 1..810: mol_type = other DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 27 - DNA, 202 bp, synthetic construct
  source 1..202: mol_type = other DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 28 - DNA, 60 bp, synthetic construct
  source 1..60: mol_type = other DNA
aggcggtcttgagacggcggatggtcgaggtgaggtgtggcaggcttgagatccagctgt

SEQ ID NO: 29 - DNA, 129 bp, synthetic construct
  source 1..129: mol_type = other DNA
cgctgttttgacctccatagtggacaccgggaccgatccagcctccgcgtctcaggggagatctcgtttagtgaaccgtcagatcctcactctcttccgcatcgctgtctgcgagggccagctgttggg

SEQ ID NO: 30 - DNA, 36 bp, synthetic construct
  source 1..36: mol_type = other DNA
ttgatattcacctggcccgatctggccatacacttg

SEQ ID NO: 31 - DNA, 22 bp, synthetic construct
  source 1..22: mol_type = other DNA
cccaggtccaagtttaaacgcc

SEQ ID NO: 32 - DNA, 77 bp, synthetic construct
  source 1..77: mol_type = other DNA
tctttgggctttctatatacattttgaaatgaggttgacaagttacctaggaaaactgtcttcctgcccgggtggca

SEQ ID NO: 33 - DNA, 1172 bp, synthetic construct
  source 1..1172: mol_type = other DNA
ctctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatg

SEQ ID NO: 34 - DNA, 1043 bp, synthetic construct
  source 1..1043: mol_type = other DNA
actgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgaagacagtcag

SEQ ID NO: 35 - protein, 431 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - DNA, 1377 bp, synthetic construct
  source 1..1377: mol_type = other DNA
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 38 - protein, 250 aa, synthetic construct
FSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKN

SEQ ID NO: 39 - DNA, 3916 bp, synthetic construct
  source 1..3916: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggtgtcatactgggatactggagtcttgctttgtgccctgctgtcctgcctcctcctgactggctccagctcgggctcagataccggtcgccccttcgtggagatgtactccgagatcccggaaattatccacatgactgaggggcgcgaacttgtgatcccctgccgggtcaccagcccgaacattactgtgactttgaagaagttccccctggacaccctgattccggatgggaagagaattatctgggattcacggaagggattcatcatcagcaacgcgacctacaaggaaattggcctcctcacttgcgaagccactgtgaacggacacttgtacaagaccaactacctgacccaccgccagaccaacaccatcatcgacgtcgtcctgtccccttcgcacgggatcgagctctcggtgggagagaagttggtgcttaactgcaccgcccggacggaactgaatgtgggaatcgacttcaactgggaatacccgtccagcaagcatcagcataagaagctggtgaaccgggacctcaagactcagtccggcagcgaaatgaagaagttcctgtcgaccctcactattgacggagtgaccagatccgaccagggcctctacacttgcgccgcttccagcggactcatgaccaagaagaacagcactttcgtgagggtgcatgagaaggacaagacccacacgtgtccgccgtgcccagccccagagctgctgggaggcccttccgtgttcctgtttccgcccaagccaaaggataccctgatgatctcaaggacccctgaggtcacatgcgtcgtggtggatgtgtcgcacgaggaccctgaagtcaaattcaattggtatgtggacggagtggaagtccacaacgcgaaaaccaagccgagagaagaacagtacaattccacctaccgggtggtgtcggtgctgactgtgctgcaccaggactggctcaacggaaaggagtacaagtgcaaggtgtccaacaaggctctgcccgcacctattgaaaagaccatctccaaggccaagggtcaacctcgcgagcctcaggtgtacactctgcctccaagccgggacgaactgactaagaaccaagtctctctgacctgtttggtgaagggcttctacccgtcagacatcgcagtggagtgggagtcaaacggtcagccggagaacaactacaaaacaaccccccccgtgctggactccgacggctccttcttcctgtactccaagcttaccgtggataagagccgctggcaacagggcaacgtgttttcctgctccgtcatgcacgaagccctgcacaaccattatacccagaagtccctgtcgctgtcccccgggaaatagtgactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 40 - DNA, 1377 bp, synthetic construct
  source 1..1377: mol_type = other DNA
atggtgtcatactgggatactggagtcttgctttgtgccctgctgtcctgcctcctcctgactggctccagctcgggctcagataccggtcgccccttcgtggagatgtactccgagatcccggaaattatccacatgactgaggggcgcgaacttgtgatcccctgccgggtcaccagcccgaacattactgtgactttgaagaagttccccctggacaccctgattccggatgggaagagaattatctgggattcacggaagggattcatcatcagcaacgcgacctacaaggaaattggcctcctcacttgcgaagccactgtgaacggacacttgtacaagaccaactacctgacccaccgccagaccaacaccatcatcgacgtcgtcctgtccccttcgcacgggatcgagctctcggtgggagagaagttggtgcttaactgcaccgcccggacggaactgaatgtgggaatcgacttcaactgggaatacccgtccagcaagcatcagcataagaagctggtgaaccgggacctcaagactcagtccggcagcgaaatgaagaagttcctgtcgaccctcactattgacggagtgaccagatccgaccagggcctctacacttgcgccgcttccagcggactcatgaccaagaagaacagcactttcgtgagggtgcatgagaaggacaagacccacacgtgtccgccgtgcccagccccagagctgctgggaggcccttccgtgttcctgtttccgcccaagccaaaggataccctgatgatctcaaggacccctgaggtcacatgcgtcgtggtggatgtgtcgcacgaggaccctgaagtcaaattcaattggtatgtggacggagtggaagtccacaacgcgaaaaccaagccgagagaagaacagtacaattccacctaccgggtggtgtcggtgctgactgtgctgcaccaggactggctcaacggaaaggagtacaagtgcaaggtgtccaacaaggctctgcccgcacctattgaaaagaccatctccaaggccaagggtcaacctcgcgagcctcaggtgtacactctgcctccaagccgggacgaactgactaagaaccaagtctctctgacctgtttggtgaagggcttctacccgtcagacatcgcagtggagtgggagtcaaacggtcagccggagaacaactacaaaacaaccccccccgtgctggactccgacggctccttcttcctgtactccaagcttaccgtggataagagccgctggcaacagggcaacgtgttttcctgctccgtcatgcacgaagccctgcacaaccattatacccagaagtccctgtcgctgtcccccgggaaatag

SEQ ID NO: 41 - protein, 458 aa, synthetic construct
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 23 bp, synthetic construct
  source 1..23: mol_type = other DNA
gagtgacaatgacatccactttg

SEQ ID NO: 43 - DNA, 22 bp, synthetic construct
  source 1..22: mol_type = other DNA
agactccagtatcccagtatga

SEQ ID NO: 44 - DNA, 24 bp, synthetic construct
  source 1..24: mol_type = other DNA
aaacttggacctgggagtggacac

SEQ ID NO: 45 - DNA, 20 bp, synthetic construct
  source 1..20: mol_type = other DNA
tgcccgcacctattgaaaag

SEQ ID NO: 46 - DNA, 18 bp, synthetic construct
  source 1..18: mol_type = other DNA
gaggctcgcgaggttgac

SEQ ID NO: 47 - DNA, 16 bp, synthetic construct
  source 1..16: mol_type = other DNA
catctccaaggccaag

(19) United States
(12) Patent Application Publication
(10) Pub. No.: US 2025/0137011 A1
(43) Pub. Date: May 1, 2025

(54) METHODS OF TREATING OCULAR NEOVASCULAR DISEASES USING AAV2 VARIANTS ENCODING AFLIBERCEPT

(71) Applicant: Adverum Biotechnologies, Inc., Redwood City, CA (US)

(72) Inventors: Kelly Hanna, Redwood City, CA (US); Kellie Schaefer-Swale, Redwood City, CA (US); Brigit Riley, Redwood City, CA (US); Ruslan Grishanin, Redwood City, CA (US); Adam Turpcu, Redwood City, CA (US); Richard Beckman, Redwood City, CA (US)

(73) Assignee: Adverum Biotechnologies, Inc., Redwood City, CA (US)

(21) Appl. No.: 18/834,864

(22) PCT Filed: Feb. 1, 2023
(86) PCT No.: PCT/US2023/061769

§ 371 Date: Jul. 31, 2024

Related U.S. Application Data
(60) Provisional application No. 63/305,838, filed on Feb. 2, 2022.
(60) Provisional application No. 63/336,191, filed on Apr. 28, 2022.
(60) Provisional application No. 63/336,789, filed on Apr. 29, 2022.
(60) Provisional application No. 63/339,795, filed on May 9, 2022.
(60) Provisional application No. 63/435,103, filed on Dec. 23, 2022.

(51) Int. Cl. C12N 15/86; A61K 38/00; A61K 48/00; C07K 14/005; C07K 14/71

(52) U.S. Cl. CPC C12N 15/86; A61K 48/0075; A61K 48/0083; C07K 14/005; C07K 14/71; A61K 38/00; C07K 2319/30; C12N 2750/14122; C12N 2750/14143

(57) ABSTRACT

Provided are methods for treating an ocular neovascular disease in an individual, comprising administering a unit dose of recombinant adeno-associated virus (rAAV) particles to an eye of the individual, wherein the rAAV particles comprise: a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

### CROSS-REFERENCE TO RELATED APPLICATIONS

This application is a 371 National Stage filing and claims the benefit under 35 U.S.C. § 120 of International Application No. PCT/US2023/061769 filed Feb. 1, 2023 which claims priority under 35 U.S.C. § 119 (e) to U.S. Provisional Application No. 63/435,103 filed Dec. 23, 2022; U.S. Provisional Application No. 63/339,795 filed May 9, 2022; U.S. Provisional Application No. 63/336,789 filed Apr. 29, 2022; U.S. Provisional Application No. 63/336,191 filed Apr. 28, 2022; and U.S. Provisional Application No. 63/305,838, filed Feb. 2, 2022; each of which is incorporated by reference in its entirety.

### STATEMENT REGARDING THE SEQUENCE LISTING

The official copy of the Sequence Listing is submitted concurrently with the specification as an WIPO Standard ST.26 formatted XML file with file name “17234.036US1.xml”, a creation date of Jul. 31, 2024, and a size of 57,344 bytes. This Sequence Listing filed via USPTO Patent Center is part of the specification and is incorporated in its entirety by reference herein.

### FIELD

The present disclosure relates to methods of treating ocular neovascular disease and disorders in an individual that comprise administering a single unit dose of a recombinant adeno associated virus (rAAV) particles encoding an anti-VEGF agent (e.g., aflibercept) to an eye of an individual.

### BACKGROUND

Age-related macular degeneration (AMD) is a degenerative ocular disease affecting the macula, a light sensitive, small area in the center of the retina that is responsible for reading and fine vision. Conditions affecting the macula reduce central vision while leaving peripheral vision intact. In severe cases, the disease can lead to central blindness. AMD is a notable cause of vision loss in the US population among persons 65 years and older, and the estimated prevalence of any AMD among persons over 40 years of age is approximately 6.5% (Klein et al., (2011) Arch Ophthalmol, 129 (1): 75-80). Neovascular or exudative or wet AMD (nAMD, wAMD, or nwAMD) is an advanced form of AMD. The hallmark of wAMD is choroidal neovascularization (CNV), which is the infiltration of abnormal blood vessels in the retina from the underlying choroid layer, resulting in retinal cell damage and central blindness. This abnormal angiogenic process is modulated by growth factors, in particular, vascular endothelial growth factor (VEGF). The standard of care of wAMD is a class of molecules that bind to and sequester VEGF, such as ranibizumab (Lucentis) and aflibercept (Eylea).

Diabetic retinopathy (DR) is a major complication of diabetes mellitus and is a leading cause of visual loss in the working age population. DR may be non-proliferative (NPDR), with no new blood vessel growth, or proliferative DR (PDR), with new abnormal blood vessel growth within the retina or choroid. Diabetic macular edema (DME) is a complication of DR and is another example of an ocular disease affecting the macula. DME affects up to 10% of people with diabetes and is caused by fluid accumulation in the macula. DME is the most frequent cause of sight loss in people with DR. Available therapies for treating DME include laser and anti-vascular endothelial growth factor (anti-VEGF) drugs such as aflibercept.

Aflibercept is a recombinant fusion protein that acts as a decoy receptor for vascular endothelial growth factor subtypes A and B (VEGF-A and VEGF-B) and placental growth factor (PGF). By binding to these ligands, aflibercept is able to prevent them from binding to vascular endothelial growth factor receptors (VEGFR), VEGFR-1 and VEGFR-2, to suppress neovascularization and decrease vascular permeability. Aflibercept consists of domain 2 of VEGFR-1 and domain 3 of VEGFR-2 fused with the Fc fragment of IgG1.

Current standard of care anti-VEGF agents such as aflibercept need to be re-administered via intravitreal (IVT) injection every 4 to 8 weeks to achieve optimal therapeutic outcomes and maintain visual acuity. Compliance with such a regimen is burdensome to patients, their caregivers, and the healthcare system, and most patients fall out of compliance with the optimal regimen over time, which is correlated with vision loss (Khanani A M, et al.). In addition, there are complications including endophthalmitis, retinal detachments, traumatic cataract, and elevated intraocular pressure (IOP); the risks of these complications are likely to increase with repeated IVT injections (Falavarjani et al., (2013) Eye (Lond), 27 (7): 787-794).

Therefore, there is a need in the art for therapies for ocular neovascular diseases such as wAMD, DR, or DME that are effective, reduce the risk of adverse effects, and are amenable to high long-term patient compliance.

### SUMMARY OF THE DISCLOSURE

In one aspect, provided herein is a method for treating an ocular neovascular disease in an individual, the method comprising administering a unit dose of about 6×10¹¹ vector genomes (vg) or less of recombinant adeno-associated virus (rAAV) particles to one eye of the individual, wherein the individual is a human, and wherein the rAAV particles comprise: (a) a nucleic acid encoding a polypeptide comprising an amino acid sequence with at least about 95% identity to the amino acid sequence of SEQ ID NO: 35 and flanked by AAV2 inverted terminal repeats (ITRs), and (b) an AAV2 capsid protein comprising, or consisting of, an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the method comprises reducing retinal fluid in an eye of the individual.

In another aspect, provided herein is a method for reducing retinal fluid in an eye of an individual with an ocular neovascular disease, the method comprising administering a unit dose of rAAV particles to one eye of the individual, wherein the individual is a human, and wherein the rAAV particles comprise: (a) a nucleic acid encoding a polypeptide comprising an amino acid sequence with at least about 95% identity to the amino acid sequence of SEQ ID NO: 35 and flanked by AAV2 inverted terminal repeats (ITRs), and (b) an AAV2 capsid protein comprising, or consisting of, an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the individual has received at least one treatment of an anti-VEGF agent in about last 12 weeks prior to administration of the unit dose of rAAV particles. In some embodiments, the amount or presence of retinal fluid in the one eye of the individual is refractory to prior treatment with an anti-VEGF agent. In some embodiments, the anti-VEGF agent is aflibercept. In some embodiments, the retinal fluid in the one eye is reduced by at least about 60%. In some embodiments, the retinal fluid in the one eye is reduced by about 80% compared to the level of retinal fluid in the one eye of the individual prior to administration of the rAAV to the individual. In some embodiments, the retinal fluid is subretinal fluid (SRF) or intraretinal fluid (IRF). In some embodiments, the unit dose of rAAV particles is about 6×10¹¹ vector genomes per eye (vg/eye) or less.

In another aspect, provided herein is a method for treating an ocular neovascular disease in an individual, the method comprising: (a) administering an anti-VEGF agent to one eye of the individual; and (b) administering a unit dose of about 6×10¹¹ vector genomes (vg) or less of recombinant adeno-associated virus (rAAV) particles to the one eye of the individual after administration of the anti-VEGF agent, wherein the individual is a human, and wherein the rAAV particles comprise: (i) a nucleic acid encoding a polypeptide comprising an amino acid sequence with at least about 95% identity to the amino acid sequence of SEQ ID NO: 35 and flanked by AAV2 inverted terminal repeats (ITRs), and (ii) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. In some embodiments, the method comprises administering the unit dose of rAAV particles to the one eye of the individual about 1 week or about 7 days after administration of the anti-VEGF agent. In some embodiments, the method comprises administering the unit dose of rAAV particles to the one eye of the individual about 1 week to about 2 weeks after administration of the anti-VEGF agent. In some embodiments, the method comprises administering the unit dose of rAAV particles to the one eye of the individual about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days after administration of the anti-VEGF agent. In some embodiments, the method comprises administering the anti-VEGF agent to the one eye of the individual on Day 1, and administering the unit dose of rAAV particles to the one eye of the individual on Day 8. In some embodiments, the anti-VEGF agent comprises aflibercept. In some embodiments, the aflibercept is administered at a dose of about 2 mg by intravitreal injection. In some embodiments, the method further comprises administering a topical steroid treatment. In some embodiments, the topical steroid treatment is a difluprednate treatment. In some embodiments, the topical steroid treatment comprises about four administrations of topical steroid per day for about four weeks, followed by about three administrations of topical steroid per day for about one week, followed by about two administrations of topical steroid per day for about one week, and followed by about one administration of topical steroid per day for about one week; timing starting with and following administration of the anti-VEGF agent. In some embodiments, the topical steroid treatment comprises about four administrations of topical steroid per day for about one month, followed by about three administrations of topical steroid per day for about one month, followed by about two administrations of topical steroid per day for about one month, and followed by about one administration of topical steroid per day for about one month; timing starting with and following administration of the anti-VEGF agent. In some embodiments, the topical steroid treatment comprises about four administrations of topical steroid per day for about one month, followed by about three administrations of topical steroid per day for about one month, followed by about two administrations of topical steroid per day for about one month, and followed by about one administration of topical steroid per day for about one month; timing starting with and following administration of the rAAV particles. In some embodiments, the topical steroid comprises difluprednate 0.05% at a dose of about 1 μg to about 3 μg. In some embodiments, the topical steroid comprises difluprednate 0.05% at a dose of about 2.5 μg.

In some embodiments that may be combined with any of the preceding embodiments, the unit dose of rAAV particles is about 6×10¹⁰ to about 2×10¹¹ vector genomes per eye (vg/eye). In some embodiments, the unit dose of rAAV particles is about 2×10¹¹ or about 6×10¹⁰ vector genomes per eye (vg/eye). In some embodiments that may be combined with any of the preceding embodiments, the unit dose of rAAV particles is between about 6×10¹⁰ to about 6×10¹¹ vector genomes per eye (vg/eye). In some embodiments that may be combined with any of the preceding embodiments, the unit dose of rAAV particles is between about 6×10¹⁰ to about 2×10¹¹ vector genomes per eye (vg/eye). In some embodiments that may be combined with any of the preceding embodiments, the unit dose of rAAV particles is between about 2×10¹¹ to about 6×10¹¹ vector genomes per eye (vg/eye). In some embodiments, the unit dose of rAAV particles is about 2×10¹¹ or about 6×10¹⁰ vector genomes per eye (vg/eye). In some embodiments, the unit dose of rAAV particles is about 2×10¹¹ vg/eye. In some embodiments, the unit dose of rAAV particles is about 6×10¹¹ vg/eye.

In some embodiments that may be combined with any of the preceding embodiments, the individual has one or more symptoms of an ocular neovascular disease in the contralateral eye.

In some embodiments that may be combined with any of the preceding embodiments, the methods provided herein further comprise administering a unit dose of rAAV particles to the contralateral eye of the individual. In some embodiments, the administering the unit dose of rAAV particles to the contralateral eye is up to about 2 weeks after administering the unit dose of rAAV particles to the one eye. In some embodiments, the administering the unit dose of rAAV particles to the contralateral eye is on the same day as the administering the unit dose of rAAV particles to the one eye; or the administering the unit dose of rAAV particles to the contralateral eye is between about 1 day to about 14 days after administering the unit dose of rAAV particles to the one eye. In some embodiments, the unit dose of rAAV particles administered to the contralateral eye of the individual comprises the same or less vector genomes per eye (vg/eye) than the unit dose of rAAV particles administered to the one eye of the individual. In some embodiments, the administering the unit dose of rAAV particles to the contralateral eye is at least about 2 weeks after administering the unit dose of rAAV particles to the one eye. In some embodiments, the unit dose of rAAV particles administered to the contralateral eye of the individual comprises more vector genomes per eye (vg/eye) than the unit dose of rAAV particles administered to the one eye of the individual.

In some embodiments that may be combined with any of the preceding embodiments, the nucleic acid comprises the nucleic acid sequence of SEQ ID NO: 40 or a sequence having at least 85% identity thereto.

In some embodiments that may be combined with any of the preceding embodiments, the polypeptide comprises the amino acid sequence of SEQ ID NO: 35. In some embodiments that may be combined with any of the preceding embodiments, the polypeptide comprises the amino acid sequence of SEQ ID NO: 41. In some embodiments, the polypeptide is aflibercept.

In some embodiments that may be combined with any of the preceding embodiments, the nucleic acid further comprises a first enhancer region, a promoter region, a 5′UTR region, a second enhancer region, and a polyadenylation site. In some embodiments, the nucleic acid comprises, in the 5′ to 3′ order: (a) a first enhancer region; (b) a promoter region; (c) a 5′UTR region; (d) a nucleic acid encoding a polypeptide comprising an amino acid sequence with at least about 95% identity to the amino acid sequence of SEQ ID NO: 35; (e) a second enhancer region; and (f) a polyadenylation site; and flanked by AAV2 inverted terminal repeats (ITRs). In some embodiments, the first enhancer region comprises a CMV sequence comprising the sequence of SEQ ID NO: 22 or a sequence having at least 85% identity thereto. In some embodiments, the promoter region comprises a CMV sequence comprising the sequence of SEQ ID NO: 23 or a sequence having at least 85% identity thereto. In some embodiments, the nucleic acid encoding a polypeptide comprises the nucleic acid sequence of SEQ ID NO: 40 or a sequence having at least 85% identity thereto. In some embodiments, the polypeptide comprises the amino acid sequence of SEQ ID NO: 35 or a sequence having at least 85% identity thereto. In some embodiments, the polypeptide comprises the amino acid sequence of SEQ ID NO: 41 or a sequence having at least 85% identity thereto. In some embodiments, the polypeptide is aflibercept. In some embodiments, the 5′UTR region comprises, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto. In some embodiments, the second enhancer region comprises a full EES sequence comprising the sequence of SEQ ID NO: 26 or a sequence having at least 85% identity thereto. In some embodiments, the polyadenylation site comprises a HGH polyadenylation site comprising the sequence of SEQ ID NO: 27 or a sequence having at least 85% identity thereto. In some embodiments, the nucleic acid further comprises (a) a first enhancer region comprising a CMV sequence comprising the sequence of SEQ ID NO: 22 or a sequence having at least 85% identity thereto; (b) a promoter region, comprising a CMV sequence comprising the sequence of SEQ ID NO: 23 or a sequence having at least 85% identity thereto; (c) a 5′UTR region comprising, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto; (d) a second enhancer region comprising a full EES sequence comprising the sequence of SEQ ID NO: 26 or a sequence having at least 85% identity thereto; and (e) a HGH polyadenylation site comprising the sequence of SEQ ID NO: 27 or a sequence having at least 85% identity thereto. In some embodiments, the nucleic acid comprises AAV ITRs flanking the elements.

In some embodiments that may be combined with any of the preceding embodiments, the nucleic acid comprises the sequence of SEQ ID NO: 39 or a sequence having at least 85% identity thereto.

In some embodiments that may be combined with any of the preceding embodiments, the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38. In some embodiments, the rAAV particles comprise an AAV2 VP1 capsid protein comprising a GH loop that comprises an amino acid sequence having any of at least 90%, at least 91%, at least 92%, at least 93%, at least 94%, at least 95%, at least 96%, at least 97%, at least 98%, at least 99%, or 100% sequence identity to SEQ ID NO: 38.

In some embodiments that may be combined with any of the preceding embodiments, the rAAV particles comprise an AAV2 VP1 capsid protein comprising the amino acid sequence of SEQ ID NO: 37 or an amino acid sequence having at least 90% sequence identity to SEQ ID NO: 37. In some embodiments, the rAAV particles comprise an AAV2 VP1 capsid protein comprising an amino acid sequence having any of at least 90%, at least 91%, at least 92%, at least 93%, at least 94%, at least 95%, at least 96%, at least 97%, at least 98%, at least 99%, or 100% sequence identity to SEQ ID NO: 37.

In some embodiments that may be combined with any of the preceding embodiments, the AAV2 capsid protein comprises, or consists of, the amino acid sequence LGETTRP (SEQ ID NO: 14) inserted between positions 587 and 588 of the AAV2 VP1 comprising the sequence of SEQ ID NO: 13. In some embodiments, the AAV2 capsid protein comprises the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the AAV2 capsid protein comprises the amino acid sequence LALGETTRPA (SEQ ID NO: 1) inserted between positions 587 and 588 of the AAV2 VP1 comprising the sequence of SEQ ID NO: 13. In some embodiments, the AAV2 capsid protein comprises the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the AAV2 capsid protein comprises the amino acid sequence LALGETTRPA (SEQ ID NO: 1) inserted between positions 587 and 588 of the AAV2 VP1 comprising the sequence of SEQ ID NO: 13.

In some embodiments that may be combined with any of the preceding embodiments, the administration of the unit dose of rAAV particles to the one eye and/or the contralateral eye is by intravitreal administration.

In some embodiments that may be combined with any of the preceding embodiments, the unit dose of rAAV particles is in a pharmaceutical formulation. In some embodiments, the pharmaceutical formulation comprises the rAAV particles, sodium chloride, sodium phosphate and a surfactant. In some embodiments, the pharmaceutical formulation comprises about 150 to about 200 mM sodium chloride, about 1 to about 10 mM monobasic sodium phosphate, about 1 to about 10 mM dibasic sodium phosphate, about 0.0005% (w/v) to about 0.005% (w/v) poloxamer 188, and about 6×10¹³ to about 6×10¹⁰ vector genomes (vg) per mL (vg/mL) of the rAAV particles, wherein the pharmaceutical formulation has a pH of about 7.0 to about 7.5. In some embodiments, the pharmaceutical formulation comprises about 180 mM sodium chloride, about 5 mM monobasic sodium phosphate, about 5 mM dibasic sodium phosphate, about 6×10¹² vg/mL of the rAAV particles, and about 0.001% (w/v) poloxamer 188, wherein the pharmaceutical formulation has a pH of about 7.3. In some embodiments, the pharmaceutical formulation comprises about 180 mM sodium chloride, about 5 mM monobasic sodium phosphate, about 5 mM dibasic sodium phosphate, about 2×10¹² vg/mL of the rAAV particles, and about 0.001% (w/v) poloxamer 188, wherein the pharmaceutical formulation has a pH of about 7.3. In some embodiments, the pharmaceutical formulation comprises about 180 mM sodium chloride, about 5 mM monobasic sodium phosphate, about 5 mM dibasic sodium phosphate, about 6×10¹¹ vg/mL of the rAAV particles, and about 0.001% (w/v) poloxamer 188, wherein the pharmaceutical formulation has a pH of about 7.3.

In some embodiments that may be combined with any of the preceding embodiments, the unit dose of rAAV particles comprises a volume of about 25 μL to about 250 μL. In some embodiments, the unit dose of rAAV particles comprises a volume of about 100 μL. In some embodiments, the unit dose of rAAV particles comprises a volume of about 30 μL. In some embodiments that may be combined with any of the preceding embodiments, the unit dose of rAAV particles administered to the one eye and/or to the contralateral eye comprises a volume of about 25 μL to about 250 μL. In some embodiments, the unit dose of rAAV particles administered to the one eye and/or to the contralateral eye comprises a volume of about 100 μL. In some embodiments, the unit dose of rAAV particles administered to the one eye and/or to the contralateral eye comprises a volume of about 50 μL. In some embodiments, the unit dose of rAAV particles administered to the one eye and/or to the contralateral eye comprises a volume of about 30 μL.

In some embodiments that may be combined with any of the preceding embodiments, the individual received prior treatment for the ocular neovascular disease with an anti-VEGF agent. In some embodiments, the individual has received 1 or 2 injections of an anti-VEGF agent in the one eye and/or in the contralateral eye prior to administration of the rAAV particles in the one eye and/or in the contralateral eye. In some embodiments, the individual has not received prior treatment for the ocular neovascular disease with an anti-VEGF agent. In some embodiments, the anti-VEGF agent is aflibercept.

In some embodiments that may be combined with any of the preceding embodiments, the ocular neovascular disease is wet age-related macular degeneration (AMD), retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy, diabetic retinal edema, or any combination thereof.

In some embodiments that may be combined with any of the preceding embodiments, the unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the oral prednisone treatment comprises administering prednisone at a dose of about 60 mg per day for a total of 6 days starting at 3 days before administration of the unit dose of rAAV particles to the one eye and/or to the contralateral eye, followed by administering prednisone at a dose of about 40 mg per day for a total of 3 days, followed by administering prednisone at a dose of about 20 mg per day for a total of 2 days, and followed by administering prednisone at a dose of about 10 mg per day for a total of 2 days. In some embodiments, the steroid treatment is a topical steroid treatment. In some embodiments, the steroid treatment is a difluprednate treatment. In some embodiments, the steroid is administered before, during and/or after administration of the unit dose of rAAV particles. In some embodiments, the steroid is administered before, during and/or after administration of the unit dose of rAAV particles to the one eye and/or to the contralateral eye.

In some embodiments that may be combined with any of the preceding embodiments, the steroid treatment is a topical steroid treatment and the topical steroid treatment is a daily steroid treatment for up to about 4 weeks, up to about 6 weeks, or up to about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid per day for about 3 weeks after administration of the unit dose of rAAV particles, followed by about 3 administrations of topical steroid per day for about 1 week, followed by about 2 administrations of topical steroid per day for about 1 week, and followed by about 1 administration of topical steroid per day for about 1 week. In some embodiments, the topical steroid treatment comprises about four administrations of topical steroid per day for about four weeks, followed by about three administrations of topical steroid per day for about one week, followed by about two administrations of topical steroid per day for about one week, and followed by about one administration of topical steroid per day for about one week; timing starting at about one week prior to administration of the unit dose of rAAV particles. In some embodiments, the topical steroid treatment comprises about four administrations of topical steroid per day for about one month, followed by about three administrations of topical steroid per day for about one month, followed by about two administrations of topical steroid per day for about one month, and followed by about one administration of topical steroid per day for about one month; timing starting at about the administration of the unit dose of rAAV particles. In some embodiments, the topical steroid treatment comprises about four administrations of topical steroid per day for about one month, followed by about three administrations of topical steroid per day for about one month, followed by about two administrations of topical steroid per day for about one month, and followed by about one administration of topical steroid per day for about one month; timing starting at about one week prior to administration of the unit dose of rAAV particles. In some embodiments, the topical steroid comprises difluprednate 0.05% at a dose of about 1 μg to about 3 μg. In some embodiments, the topical steroid comprises difluprednate 0.05% at a dose of about 2.5 μg.

In some embodiments, the ocular neovascular disease is wet age-related macular degeneration (wAMD).

In some embodiments that may be combined with any of the preceding embodiments, the administering the unit dose of rAAV particles to the one eye and/or to the contralateral eye of the individual results in maintenance or a decrease of retinal thickness compared to the retinal thickness prior to administration of the unit dose of rAAV particles. In some embodiments, the maintenance or the decrease of retinal thickness compared to the retinal thickness prior to administration of the unit dose of rAAV particles is present at about 30 weeks, about 34 weeks, about 44 weeks, about 6 months, about 1 year, about 1.5 years, about 2 years, about 3 years, about 5 years, about 10 years, or more after administration of the unit dose of rAAV particles to the one eye and/or to the contralateral eye of the individual. In some embodiments, the administering the unit dose of rAAV particles to the one eye and/or to the contralateral eye of the individual results in a decrease in retinal thickness compared to the retinal thickness prior to administration of the unit dose of rAAV particles. In some embodiments, the decrease in retinal thickness is at least about 10% compared to the retinal thickness prior to administration of the unit dose of rAAV particles. In some embodiments, retinal thickness is central subfield thickness (CST) or central retinal thickness (CRT).

In some embodiments that may be combined with any of the preceding embodiments, the administering the unit dose of rAAV particles to the one eye and/or to the contralateral eye of the individual results in maintenance or a decrease in macular volume compared to the macular volume prior to administration of the unit dose of rAAV particles. In some embodiments, the maintenance or the decrease in macular volume compared to the macular volume prior to administration of the unit dose of rAAV particles is present at about 30 weeks, about 34 weeks, about 44 weeks, about 6 months, about 1 year, about 1.5 years, about 2 years, about 3 years, about 5 years, about 10 years, or more after administration of the unit dose of rAAV particles to the one eye and/or to the contralateral eye of the individual. In some embodiments, the administering the unit dose of rAAV particles to the one eye and/or to the contralateral eye of the individual results in a decrease in macular volume compared to the macular volume prior to administration of the unit dose of rAAV particles. In some embodiments, the decrease in macular volume is at least about 10% compared to the macular volume prior to administration of the unit dose of rAAV particles.

In some embodiments that may be combined with any of the preceding embodiments, the administering the unit dose of rAAV particles to the one eye and/or to the contralateral eye of the individual results in maintenance or an improvement of visual acuity compared to the visual acuity prior to administration of the unit dose of rAAV particles. In some embodiments, the maintenance or the improvement of visual acuity compared to the visual acuity prior to administration of the unit dose of rAAV particles is present at about 30 weeks, about 34 weeks, about 44 weeks, about 6 months, about 1 year, about 1.5 years, about 2 years, about 3 years, about 5 years, about 10 years, or more after administration of the unit dose of rAAV particles to the one eye and/or to the contralateral eye of the individual. In some embodiments, the administering the unit dose of rAAV particles to the one eye and/or to the contralateral eye of the individual results in an improvement of visual acuity compared to the visual acuity prior to administration of the unit dose of rAAV particles. In some embodiments, visual acuity is best corrected visual acuity (BCVA).

In some embodiments that may be combined with any of the preceding embodiments, administration of a single unit dose of rAAV particles to the one eye and/or to the contralateral eye of the individual provides a therapeutic benefit (e.g., treatment of an ocular neovascular disease, reduction of retinal fluid, maintenance or a decrease of retinal thickness, maintenance or a decrease in macular volume, and/or maintenance or an improvement of visual acuity). In some embodiments, administration of a single unit dose of rAAV particles to the one eye and/or to the contralateral eye of the individual provides a therapeutic benefit that is present at about 30 weeks or more, about 34 weeks or more, about 44 weeks or more, about 6 months or more, about 1 year or more, about 1.5 years or more, or about 2 years, about 3 years, about 5 years, about 10 years, or more after administration of the unit dose of rAAV particles to the one eye and/or to the contralateral eye of the individual.

In some embodiments that may be combined with any of the preceding embodiments, administration of the unit dose of rAAV particles to the one eye and/or to the contralateral eye of a plurality of individuals results in at least about 50% of the individuals in the plurality not requiring an anti-VEGF rescue treatment. In some embodiments, at least about 50% of the individuals in the plurality do not require an anti-VEGF rescue treatment for at least about 20 weeks, at least about 36 weeks, at least about 52 weeks, at least about 56 weeks, or more after administration of the unit dose of rAAV particles.

In some embodiments that may be combined with any of the preceding embodiments, administration of a single unit dose of rAAV particles to the one eye and/or to the contralateral eye of a plurality of individuals results in at least about 67% of the individuals in the plurality not requiring an anti-VEGF rescue treatment. In some embodiments, at least about 67% of the individuals in the plurality do not require an anti-VEGF rescue treatment for at least about 20 weeks, at least about 36 weeks, at least about 52 weeks, at least about 60 weeks, at least about 64 weeks, or at least about 66 weeks after administration of the unit dose of rAAV particles.

In some embodiments that may be combined with any of the preceding embodiments, administration of the unit dose of rAAV particles to the one eye and/or to the contralateral eye of a plurality of individuals results in at least about 78% of the individuals in the plurality not requiring an anti-VEGF rescue treatment. In some embodiments, at least about 78% of the individuals in the plurality do not require an anti-VEGF rescue treatment for at least about 20 weeks, at least about 36 weeks, or more after administration of the unit dose of rAAV particles.

In some embodiments that may be combined with any of the preceding embodiments, administration of the unit dose of rAAV particles to the one eye and/or to the contralateral eye of a plurality of individuals results in 100% of the individuals in the plurality not requiring an anti-VEGF rescue treatment. In some embodiments, 100% of the individuals in the plurality do not require an anti-VEGF rescue treatment for at least about 64 weeks, at least about 68 weeks, at least about 72 weeks, at least about 76 weeks, at least about 80 weeks, at least about 84 weeks, or more after administration of the unit dose of rAAV particles.

In some embodiments that may be combined with any of the preceding embodiments, administration of the unit dose of rAAV particles to the one eye and/or to the contralateral eye of a plurality of individuals results in a reduction in the annualized anti-VEGF injection rate of at least about 80%, at least about 85%, at least about 87%, at least about 90%, at least about 95%, at least about 99%, or 100% compared to the annualized anti-VEGF injection rate prior to administration of the unit dose of rAAV particles.

In some embodiments, the ocular neovascular disease is diabetic macular edema (DME). In some embodiments, the administering the unit dose of rAAV particles to the one eye and/or to the contralateral eye of the individual results in a 2-step or in a 3-step improvement in Diabetic Retinopathy Severity Scale (DRSS). In some embodiments, the administering the unit dose of rAAV particles to the one eye and/or to the contralateral eye of the individual results in a 2-step improvement in Diabetic Retinopathy Severity Scale (DRSS). In some embodiments, the administering the unit dose of rAAV particles to the one eye and/or to the contralateral eye of the individual results in a 3-step improvement in Diabetic Retinopathy Severity Scale (DRSS).

In an embodiment, methods for treating an ocular neovascular disease in an individual are provided. The methods comprise administering a unit dose of about 2×10¹¹ vector genomes (vg) or less of recombinant adeno-associated virus (rAAV) particles to one eye of an individual, wherein the individual is human and wherein the rAAV particles comprise (a) a nucleic acid encoding a polypeptide comprising an amino acid sequence with at least about 95% identity to the amino acid sequence of claim 35 and flanked by AAV2 inverted terminal repeats (ITRs) and (b) an AAV2 capsid protein comprising an amino acid sequence LGETTRP (SEQ ID NO:14) inserted between positions 587 and 588 of the capsid protein, wherein the amino acid residue numbering corresponds to an AAV2 VP1 capsid protein. In various aspects of the methods, retinal fluid in the eye of an individual with an ocular neovascular disease is reduced.

In an embodiment, methods for reducing retinal fluid in an eye of an individual with an ocular neovascular disease are provided. The methods comprise administering a unit dose of rAAV particles to one eye of the individual, wherein the individual is human, and wherein the rAAV particles comprise: (a) a nucleic acid encoding a polypeptide comprising an amino acid sequence with at least about 95% identity to the amino acid sequence of SEQ ID NO:35 and flanked by AAV2 inverted terminal repeats (ITRs), and (b) an AAV2 capsid protein comprising an amino acid sequence LGETTRP (SEQ ID NO:14) inserted between positions 587 and 588 of the capsid protein, wherein the amino acid residue numbering corresponds to an AAV2 VP1 capsid protein. In aspects of the methods, the individual has received at least one treatment of an anti-VEGF agent in about 12 weeks prior to administration of the unit dose of rAAV particles. In aspects of various methods, the amount or presence of retinal fluid in the one eye of the individual is refactory to prior treatment with an anti-VEGF agent. In further aspects of the methods, the anti-VEGF agent is aflibercept. In aspects of any of the above methods, the retinal fluid in the one eye is reduced by about 80% compared to the level of retinal fluid in the one eye of the individual prior to administration of the rAAV to the individual. In aspects of any of the above methods, the retinal fluid is subretinal fluid (SRF) or intraretinal fluid (IRF).

Methods for treating an ocular neovascular disease in an individual are provided. The methods comprise (a) administering an anti-VEGF agent to one eye of the individual and (b) administering a unit dose of about 6×10¹¹ vector genomes (vg) or less of recombinant adeno-associated virus (rAAV) particles to the one eye of the individual after administration of the anti-VEGF agent, wherein the individual is human, and wherein the rAAV particles comprise (i) a nucleic acid encoding a polypeptide comprising an amino acid sequence with at least about 95% identity to the amino acid sequence of SEQ ID NO:35 and flanked by AAV2 inverted terminal repeats (ITRs) and (ii) an AAV capsid protein comprising an amino acid sequence LGETTRP (SEQ ID NO:14) inserted between positions 587 and 588 of the capsid protein, wherein the amino acid residue numbering corresponds to an AAV2 VP1 capsid protein. Aspects of the methods comprise administering the unit dose of rAAV particles to the one eye of the individual about 1 week or about 7 days after the administration of the anti-VEGF agent. Aspects of the methods comprise administering the anti-VEGF agent to the one eye of the individual on Day 1, and administering the unit dose of rAAV particles to the one eye of the individual on Day 8. In aspects of the methods, the anti-VEGF agent comprises aflibercept. In aspects of the method, the aflibercept is administered at a dose of about 2 mg by intravitreal injection.

In aspects of the above methods, the unit dose of rAAV particles is between about 6×10¹⁰ to about 6×10¹¹ vector genomes per eye (vg/eye). In aspects of the above methods, the unit dose of rAAV particles is between about 6×10¹⁰ to about 2×10¹¹ vector genomes per eye (vg/eye). In some aspects of the above methods, the unit dose of rAAV particles is about 2×10¹¹ vector genomes per eye (vg/eye). In other aspects of the above methods, the unit dose of rAAV particles is about 6×10¹¹ vector genomes per eye (vg/eye).

In aspects of any of the above methods, the individual has one or more symptoms of an ocular neovascular disease in the contralateral eye. Various aspects of the above methods further comprise administering a unit dose of rAAV particle to the contralateral eye of the individual. Administering the unit dose of rAAV particles to the contralateral eye is up to about 2 weeks after administering the unit dose of rAAV particles to the one eye in some aspects. In some aspects, administering the unit dose of rAAV particle to the contralateral eye is on the same day as the administering the unit dose of rAAV particles to the one eye or the administering the unit dose of rAAV particles to the contralateral eye is between about 1 day to about 14 days after administering the unit dose of rAAV particles to the one eye. In some aspects, the unit dose of rAAV particles administered to the contralateral eye of the individual comprises the same or less vector genomes per eye (vg/eye) than the unit dose of rAAV particles administered to the one eye of the individual. In some aspects, administering the unit dose of rAAV particles to the contralateral eye is at least about 2 weeks after administering the unit dose of rAAV particles to the one eye. In some aspects of the above methods, the unit dose of rAAV particles administered to the contralateral eye of the individual comprises more vector genomes per eye (vg/eye) than the unit dose of rAAV particles administered to the one eye of the individual.

In aspects of any of the above methods, the nucleic acid comprises the nucleic acid sequence of SEQ ID NO:40 or a sequence having at least 85% identity thereto. In aspects of the above methods, the polypeptide comprises the amino acid sequence of SEQ ID NO: 35. In aspects of any of the above methods, the polypeptide comprises the amino acid sequence of SEQ ID NO:41. In aspects of any of the methods, the polypeptide is aflibercept. In aspects of the methods, the nucleic acid further comprises a first enhancer region, a promoter region, a 5′UTR region, a second enhancer region and a polyadenylation site. In aspects of the methods, the nucleic acid comprises, in the 5′ to 3′ order: (a) a first enhancer region (b) a promoter region; (c) a 5′ UTR region; (d) a nucleic acid encoding a polypeptide comprising an amino acid sequence with at least about 95% identity to the amino acid sequence of SEQ ID NO:35; (e) a second enhancer region; and (f) a polyadenylation site, and flanked by AAV2 inverted terminal repeats (ITRs). In some aspects, the first enhancer region comprises a CMV sequence comprising the sequence of SEQ ID NO:22 or a sequence having at least 85% identity thereto. In some aspects, the promoter region comprises a CMV sequence comprising the sequence of SEQ ID NO:23 or a sequence having at least 85% identity thereto. In some aspects of the methods, the nucleic acid encoding a polypeptide comprises the nucleic acid sequence of SEQ ID NO:40 or a sequence having at least 85% identity thereto. In various aspects, the polypeptide comprises the amino acid sequence of SEQ ID NO:35 or a sequence having at least 95% identity thereto. In various aspects, the polypeptide comprises the amino acid sequence of SEQ ID NO:41 or a sequence having at least 95% identity thereto. In some aspects, the polypeptide is aflibercept. In various aspects, the 5′UTR region comprises, in 5′ to 3′ order, a TPL sequence comprising the sequence of SEQ ID NO:24 or a sequence having at least 85% identity thereto, and an eMLP sequence comprising the sequence of SEQ ID NO:25 or a sequence having at least 85% identity thereto. In some aspects of the methods, the second enhancer region comprises a full EES sequence comprising the sequence of SEQ ID NO: 26 or a sequence having at least 85% identity thereto. In certain aspects of the methods, the polyadenylation site comprises an HGH polyadenylation site comprising the sequence of SEQ ID NO:27 or a sequence having at least 85% identity thereto.

In an aspect of the methods, the nucleic acid further comprises (a) a first enhancer region comprising a CMV sequence comprising the sequence set forth in SEQ ID NO:22 or a sequence having at least 85% identity thereto; (b) a promoter region, comprising a CMV sequence comprising the sequence of SEQ ID NO:23 or a sequence having at least 85% identity thereto, (c) a 5′ UTR comprising in 5′ to 3′ order, a TPL sequence comprising the sequence of SEQ ID NO:24 or a sequence having at least 85% identity thereto, and an eMLP sequence comprising the sequence of SEQ ID NO:25 or a sequence having at leat 85% identity thereto, (d) a second enhancer region comprising a full EES sequence comprising the sequence of SEQ ID NO:26 or a sequence having at least 85% identity thereto, and (e) a HGH polyadenylation site comprising the sequence of SEQ ID NO:27 or a sequence having at least 85% identity thereto.

In aspects of any of the methods, the nucleic acid comprises the sequence of SEQ ID NO: 39 or a sequence having at least 85% identity thereto.

In aspects of any of the above methods, the AAV2 capsid protein comprises the amino acid sequence LGETTRPA (SEQ ID NO:1) inserted between positions 587 and 588 of the capsid protein, wherein the amino acid residue numbering corresponds to an AAV2 VP1 capsid protein comprising the sequence of SEQ ID NO:13. In aspects of any of the above methods, the AAV2 capsid protein comprises the amino acid sequence LALGETTRPA (SEQ ID NO:1) inserted between positions 587 and 588 of the capsid protein, wherein the amino acid residue numbering corresponds to an AAV2 VP1 capsid protein comprising the sequence of SEQ ID NO: 13.

In aspects of any of the above methods, the rAAV particles comprise an AAV2 VP1 capsid protein comprising a GH loop that comprises the amino acid sequence of SEQ ID NO: 38 or an amino acid sequence having at least 90% sequence identity to SEQ ID NO:38.

Aspects of any of the above methods, wherein the administration of the unit dose of rAAV particles to the one eye and/or the contralateral eye is by intravitreal administration are provided.

In aspects of any of the above methods, the unit dose of rAAV particles is in a pharmaceutical formulation. In some aspects, the pharmaceutical formulation comprises the rAAV particles, sodium chloride, sodium phosphate and a surfactant. In an aspect of the above methods, the pharmaceutical composition comprises about 150 to about 200 mM sodium chloride, about 1 to about 10 mM monobasic sodium phosphate, about 1 to about 10 mM dibasic sodium phosphate, about 0.0005% (w/v) to about 0.0005% (w/v) poloxamer 188 and about 6×10¹³ to about 6×10¹⁰ vector genomes (vg) per mL (vg/ml) of the rAAV particles wherein the pharmaceutical formulation has a pH of about 7.0 to about 7.5. In some aspects, the pharmaceutical composition comprises about 180 mM sodium chloride, about 5 mM monobasic sodium phosphate, about 5 mM dibasic sodium phosphate, about 6×10¹² vg/ml of the rAAV particles and about 0.001% (w/v) poloxamer 188, wherein the pharmaceutical formulation has a pH of about 7.3. In some aspects, the pharmaceutical composition comprises about 180 mM sodium chloride, about 5 mM monobasic sodium phosphate, about 5 mM dibasic sodium phosphate, about 6×10¹¹ vg/ml of the rAAV particles and about 0.001% (w/v) poloxamer 188, wherein the pharmaceutical formulation has a pH of about 7.3.

In aspects of any of the above methods, the unit dose of rAAV particles administered to the one eye and or to the contralateral eye is in a volume of about 25 μl to about 250 μl. In some aspects, the unit dose of rAAV particles administered to one eye and/or to the contralateral eye comprises a volume of about 100 μl. In some aspects, the unit dose of rAAV particles administered to one eye and/or to the contralateral eye comprises a volume of about 30 μl.

In aspects of the above methods, the individual received prior treatment for the ocular neovascular disease with an anti-VEGF agent. In some aspects, the individual has received 1 or 2 injections of an anti-VEGF agent in the one eye and/or in the contralateral eye prior to administration of the rAAV particles in the one eye and/or in the contralateral eye.

In aspects of some of the above methods, the individual has not received prior treatment for the ocular neovascular disease with an anti-VEGF agent.

In aspects of any one of the above methods, the anti-VEGF agent is aflibercept.

In aspects of any one of the above methods, the ocular neovascular disease is wet age-related macular degeneration (AMD), retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemia retinopathy, diabetic retinal edema or any combination thereof.

In aspects of the above methods, the unit dose of rAAV particles is administered in combination with steroid treatment. In certain aspects, the steroid treatment is a corticosteroid treatment. In some aspects, the steroid treatment is a systemic steroid treatment. In various aspects, the steroid treatment is an oral steroid treatment. In some aspects, the steroid treatment is a prednisone treatment. In certain aspects of the methods, the steroid treatment is a topical steroid treatment. In aspects of the methods, the steroid treatment is a difluprednate treatment. In aspects of the above methods, the steroid is administered before, during and/or after administration of the unit dose of rAAV particles to the one eye and/or to the contralateral eye.

In aspects of some methods, the steroid treatment is a topical steroid treatment and the topical steroid treatment is a daily steroid treatment for up to about 4 weeks, up to about 6 weeks, or up to about 8 weeks from administering the unit dose of rAAV particles. In some aspects of the above method, the topical steroid treatment comprises about four administrations of a topical steroid per day on about week 1, about three administrations of topical steroid per day on about week 2, about two administrations of topical steroid per day on about week 3, and about one administration per day of topical steroid on about week 4; timing starting with and following administration of the unit dose of rAAV particles. In some aspects of the methods, the topical steroid treatment comprises about four administrations of topical steroid per day for about three weeks after administration of the unit dose of rAAV particles, followed by about 3 administrations of topical steroid per day for about 1 week, followed by about 2 administrations of topical steroid per day for about 1 week and followed by about 1 administration of topical steroid per day for about 1 week. In some aspects of the methods, the topical steroid treatment comprises about four administrations of topical steroid per day for about four weeks, followed by about three administrations of topical steroid per day for about one week, followed by about two administrations of topical steroid per day for about one week, followed by about one administration of topical steroid per day for about one week; timing starting at about one week prior to administration of the unit dose of rAAV particles.

In some aspects of the methods, the methods further comprise administering a topical steroid treatment. In aspects of the methods, the topical steroid treatment is a difluprednate treatment. In aspects of the above methods, the topical steroid treatment comprises about four administrations of topical steroid per day for about four weeks, followed by about three administrations of topical steroid per day for about one week, followed by about two administrations of topical steroid per day for about one week, followed by about one administration of topical steroid per day for about one week; timing starting with and following administration of an anti-VEGF agent. In aspects of the above methods, the topical steroid comprises difluprednate 0.05% at a dose of about 1 μg to about 3 μg. In some aspects of the above methods, the topical steroid comprises difluprednate 0.05% at a dose of about 2.5 μg.

In aspects of any of the above methods administering the unit dose of rAAV particles to the one eye and/or to the contralateral eye of the individual results in maintenance of or a decrease of retinal thickness compared to the retinal thickness prior to administration of the unit dose of rAAV particles. In some aspects, the administering the unit dose of rAAV particles to the one eye and/or to the contralateral eye of the individual results in a decrease in retinal thickness compared to the retinal thickness prior to administration of the unit dose of rAAV particles. In certain aspects of the methods, the decrease in retinal thickness is at least about 10% compared to the retinal thickness prior to administration of the unit dose of rAAV particles. In aspects of certain methods, the retinal thickness is central subfield thickness (CST) or central retinal thickness (CRT).

In aspects of any of the above methods, administering the unit dose of rAAV particles to the one eye and/or to the contralateral eye of the individual results in maintenance or a decrease in macular volume compared to the macular volume prior to administration of the unit dose of rAAV particles. In an aspect of the methods, administering the unit dose of rAAV particles to the one eye and/or to the contralateral eye of the individual results in a decrease in macular volume compared to the macular volume prior to administration of the unit dose of rAAV particles. In aspect of the methods, the decrease in macular volume is at least about 10% compared to the macular volume prior to administration of the unit dose of rAAV particles.

In aspects of any of the above methods, the administering the unit dose of rAAV particles to the one eye and/or to the contralateral eye of the individual results in maintenance or an improvement of visual acuity compared to the visual acuity prior to administration of the unit dose of rAAV particles. In some aspects of the methods, administering the unit dose of rAAV particles to the one eye and/or to the contralateral eye of the individual results in an improvement of visual acuity compared to the visual acuity prior to administration of the unit dose of rAAV particles. In aspects of the methods, visual acuity is best corrected visual acuity (BCVA).

In aspects of any of the above methods, administration of the unit dose of rAAV particles to the one eye and/or to the contralateral eye of a plurality of individuals results in at least about 50% of the individuals in the plurality not requiring an anti-VEGF rescue treatment. In some aspects of the methods, at least about 50% of the individuals in the plurality do not require an anti-VEGF rescue treatment for at least about 20 weeks, at least about 36 weeks, at least about 52 weeks, at least about 56 weeks or more after administration of the unit dose of rAAV particles.

In aspects of any of the above methods, administration of the unit dose of rAAV particles to the one eye and/or to the contralateral eye of a plurality of individuals results in at least about 67% of the individuals in the plurality not requiring an anti-VEGF rescue treatment. In some aspects of the methods, at least about 67% of the individuals in the plurality do not require an anti-VEGF rescue treatment for at least about 20 weeks, at least about 36 weeks, at least about 52 weeks, at least about 60 weeks, at least about 64 weeks or at least about 66 weeks after administration of the unit dose of rAAV particles.

In aspects of the above methods, administration of the unit dose of rAAV particles to the one eye and/or to the contralateral eye of a plurality of individuals results in at least about 78% of the individuals in the plurality not requiring an anti-VEGF rescue treatment. In various aspects at least about 78% of the individuals in the plurality do not require an anti-VEGF rescue treatment for at least about 20 weeks, at least about 36 weeks or mor after administration of the unit dose of rAAV particles.

In aspects of the above methods, administration of the unit dose of rAAV particles to the one eye and/or to the contralateral eye of a plurality of individuals results in 100% of the individuals in the plurality not requiring an anti-VEGF rescue treatment. In aspects of the methods, 100% of the individuals in the plurality do not require an anti-VEGF rescue treatment for at least about 64 weeks, at least about 68 weeks, at least about 72 weeks, at least about 76 weeks, at least about 80 weeks, at least about 84 weeks or more after administration of the unit dose of rAAV particles.

Aspects of any of the above methods provide administration of the unit dose of rAAV particles to the one eye and/or to the contralateral eye of a plurality of individuals that results in a reduction in the annualized anti-VEFG injection rate of at least about 80%, at least about 85%, at least about 87%, at least about 90%, at least about 95% at least about 99% or 100% compared to the annualized anti-VEGF injection rate prior to administration of the unit dose of rAAV particles.

In aspects of various methods, the ocular neovascular disease is wet age-related macular degeneration (AMD).

In aspects of various methods, the ocular neovascular disease is diabetic macular edema (DME). In various aspects, administering the unit dose of rAAV particles to the one eye and/or to the contralateral eye of the individual results in a 2-step or in a 3-step improvement in Diabetic Retinopathy Severity Score (DRSS).

Embodiments of the application provide a unit dose of about 6×1011 vector genomes (vg) or less of recombinant adeno-associated virus (rAAV) particles for use in a method for treating an ocular neovascular disease in an individual, the method comprising administering the unit dose to one eye of the individual, wherein the individual is a human and wherein the rAAV particle comprise (a) a nucleic acid encoding a polypeptide comprising an amino acid sequence with at least about 95% identity to the amino acid sequence of SEQ ID NO:35 and flanked by AAV2 inverted terminal repeats (ITRs) and (b) an AAV2 capsid protein comprising an amino acid sequence LGETTRP (SEQ ID NO:14) inserted between positions 587 and 588 of the capsid protein, wherein the amino acid residue numbering corresponds to an AAV2 VP1 capsid protein.

An embodiment of the application provides a unit dose of rAAV particles for use in a method of reducing retinal fluid in an eye of an individual with an ocular neovascular disease, the method comprising administering the unit dose to one eye of the individual, wherein the individual is human and wherein the rAAV particles comprise (a) a nucleic acid encoding a polypeptide comprising an amino acid sequence with at least about 95% identity to the amino acid sequence of SEQ ID NO:35 and flanked by AAV2 inverted terminal repeats (ITRs) and (b) an AAV2 capsid protein comprising an amino acid sequence LGETTRP (SEQ ID NO:14) inserted between positions 587 and 588 of the capsid protein, wherein the amino acid residue numbering corresponds to an AAV2 VP1 capsid protein. Aspects of the above unit doses for use in an indicated method wherein the method further comprises administering an anti-VEGF agent to one eye of the individual before administration of the unit dose.

### INCORPORATION BY REFERENCE

All references cited herein, including patent applications and publications, are incorporated by reference in their entirety.

### BRIEF DESCRIPTION OF THE DRAWINGS

FIG. 1 provides a summary of collection eyes utilized In Life Humor Collections at the indicated time points (30 days, 60 days and 98 days) in the aqueous humor (AH) or vitreous humor (VH). The eye or eyes from which the collection was made is indicated. “OD” signifies the ocular dexter or right eye. “OS” signifies the ocular sinister or left eye. “OU” signifies oculus uterque or both eyes. The subject animal identifier and group number are also indicated.

FIG. 2 provides a table summarizing aflibercept levels obtained in the Vitreous Humor at the indicated time points and in the indicated eye or eyes of the subject. BLOQ signifies “below the level of quantitation”. Dosing is indicated in vg/eye. Eyes were treated with either 3E10 or 1E11 viral genomes (vg)/treated eye. Subject animals are identified.

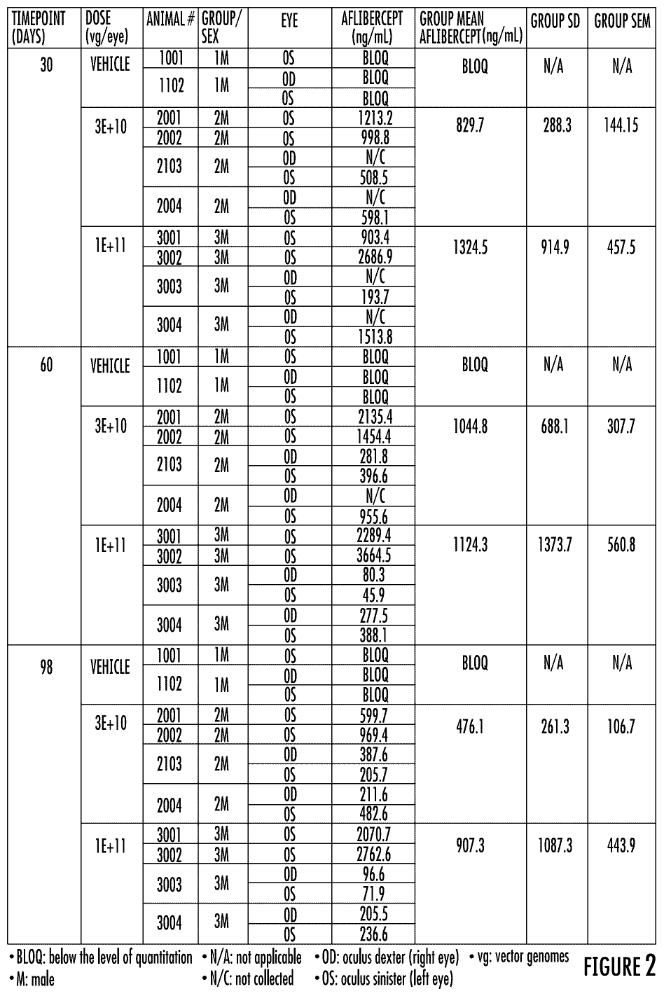

FIG. 3 provides a table summarizing aflibercept levels obtained in the aqueous humor at the indicated time points and in the indicated eye or eyes of the subject. BLOQ signifies “below the level of quantitation”. Dosing is indicated in vg/eye. Eyes were treated with either 3E10 or 1E11 viral genomes (vg)/treated eye. Subject animals are identified.

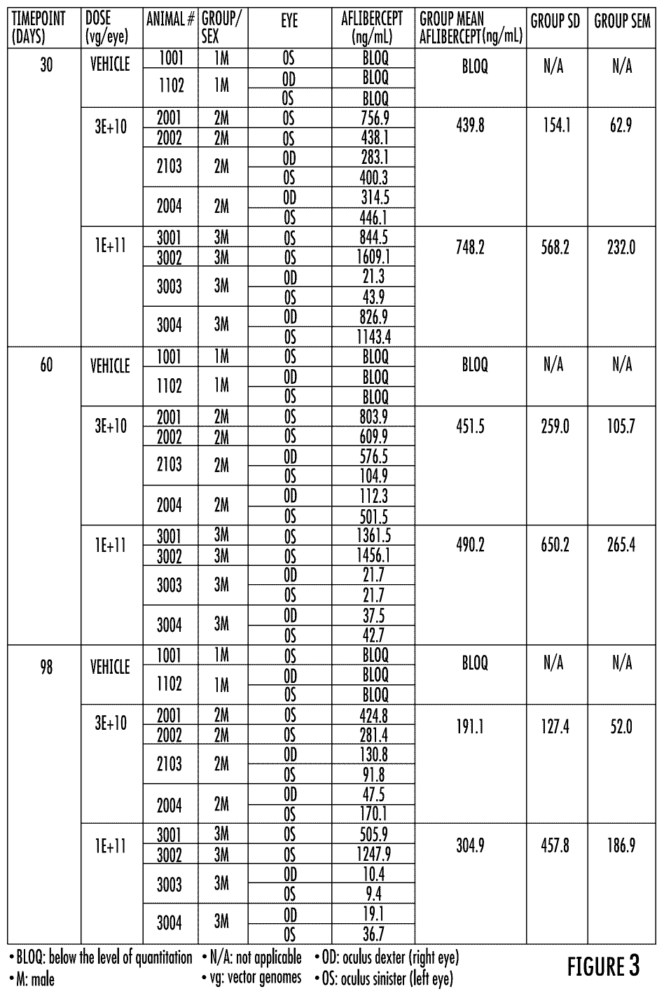

FIG. 4 provides charts summarizing aqueous aflibercept concentrations in either the aqueous humor or the vitreous humor. Peak aqueous Aflibercept values exceeded or approached 500 ng/mL after a dose of 3.00E10 vg/mL. Animals were treated with a dose of 3.00E10 (gray) or 1.00E11 (black) vg/mL. Aqueous and vitreous humor were collected at Days 30, 60, and 90 post-dose. Aflibercept levels were measured using an Aflibercept ELISA. The peak Aflibercept value detected in aqueous and vitreous humors for each animal is represented here. Black horizonal bars denote group mean, black vertical bars denote SEM, and asterisks denote outlier samples which are hypothesized to be the result of anti-drug antibodies.

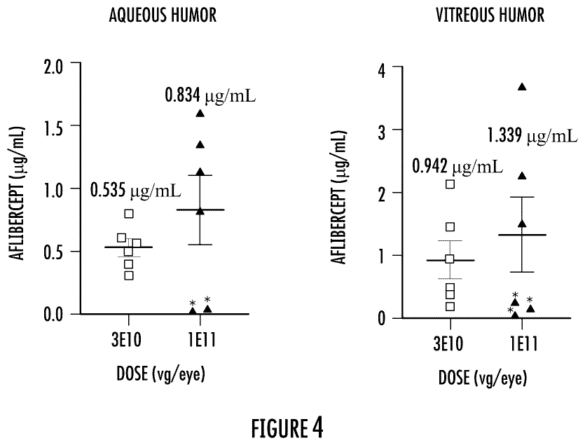

FIG. 5 provides charts summarizing historical data of aqueous humor shows a flat dose response over 3 logs of dosing levels. The chart on the right is in logarithmic scale.

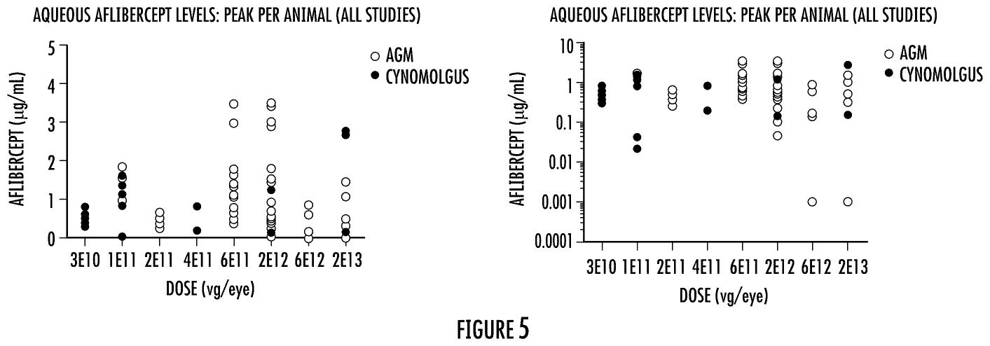

FIG. 6 provides graphs summarizing the distribution of ADVM-022 vector genomes (vg) in tissues of non-human primates (NHPs) dosed with ADVM-022 at 3×10¹⁰ (left) and 1×10¹¹ vg/eye (right). Tissues were collected on Day 97 post-dose. “*” denotes data from right (R) and left (L) organs combined; LN denotes lymph node; LGN denotes lateral geniculate nucleus. Horizontal bars correspond to geometric means. Dosing impacts the distribution of ADVM-022 vg in some tissues more than other tissues.

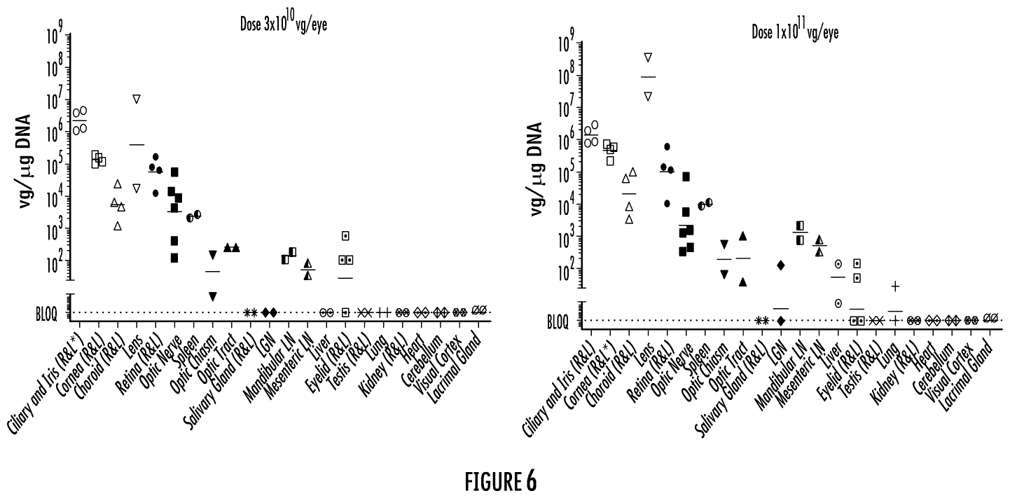

FIG. 7 provides graphs summarizing the distribution of ADVM-022 expressed mRNA in tissues of non-human primates (NHPs) dosed with ADVM-022 at 3×10¹⁰ (left) and 1×10¹¹ vg/eye (right). Tissues were collected on Day 97 post-does. “*” denotes data from right (R) and left (L) organs combined; LN denotes lymph node; LGN denotes lateral geniculate nucleus. Horizontal bars correspond to geometric means. Altered doses impact the expression of ADVM-022 mRNA in certain tissues.

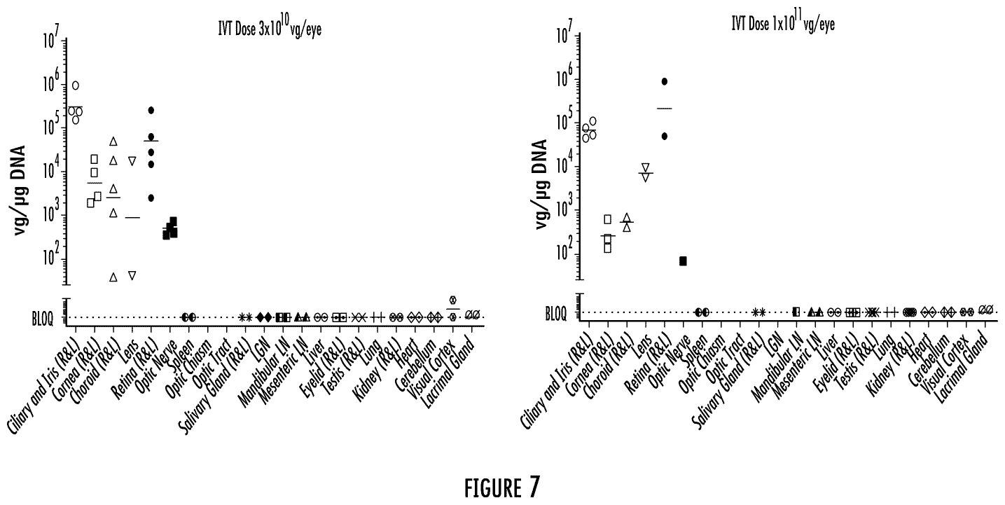

FIG. 8 provides graphs summarizing the distribution of ADVM-022 vector genomes (vg) in whole blood of non-human primates (NHPs) dosed with ADVM-022 at 3×10¹⁰ (left) and 1×10¹¹ vg/eye (right). The time of the whole blood collection is indicate on the x-axis. Whole blood was collected at BL (Baseline), 8 h (0.33 days), 48 h (2 days), 144 h (6 days), 336 h (14 days), 43 and 87 days post-dose.

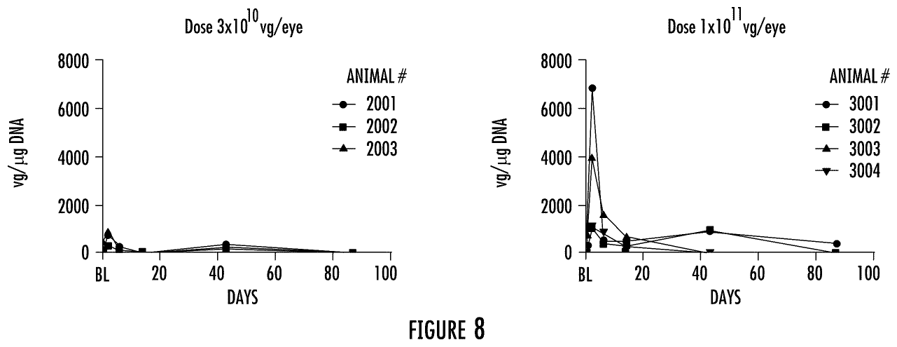

FIG. 9 provides a visual representation of ADVM-022 vector genome biodistribution in the NHP eye dosed with 3E10 vg/eye. Arrowheads point to some of ADVM-022 DNA-positive cells and extracellular particles in photomicrographs of samples obtained from the indicated region. In the Macula (region A), intracellular vector DNA is detected in the macular retinal ganglion cells and in the nuclear cell layer. In the Mid-retina (region B), no intracellular vector DNA is detected; vector DNA is detected as particles associated with inner limiting membrane. In the Peripheral retina (region C), intracellular vector DNA is detected in the retinal ganglion cell layer and in the nuclear cell layer. In the Ciliary body (region D), intracellular vector DNA is detected in some nonpigmented epithelial cells of ciliary processes.

FIG. 10 provides a visual representation of ADVM-022 transgene mRNA biodistribution in the NHP eye dosed with 3E10 vg/eye. Arrowheads point to some of ADVM-022 positive cells in photomicrographs of samples obtained from the indicated region. In the Macula (region A), transgene mRNA is detected in the macular retinal ganglion cells and in the nuclear cell layer. In the Peripheral retina (region b), transgene mRNA is detected in the retinal ganglion cell layer and in the nuclear cell layer. In the Ciliary body (region c), transgene mRNA is detected in some nonpigmented epithelial cells of ciliary processes and in the area of ciliary muscle.

FIG. 11 is a diagram depicting the design of the Phase 2 study of AAV2.7m8-aflibercept in anti-VEGF treatment-experienced patients with neovascular (wet) age-related macular degeneration (nAMD) as described in Example 1 herein. This study includes 8 study arms (Arms 1A, 1B, 2A, 2B, 3A, 3B, 4A, and 4B), with participants in each arm receiving a single dose of AAV2.7m8-aflibercept at a unit dose of 2×10¹¹ vg/eye or 6×10¹⁰ vg/eye, accompanied by a prophylactic corticosteroid treatment regimen (topical; IVT; topical+oral; or IVT+oral). The study includes a screening period (14-21 days), and a 50-week study period following dosing with AAV2.7m8-aflibercept. IVT=intravitreal; nAb=neutralizing antibody; AFL=aflibercept; D=Day; W=Week; q4W=every four weeks; CST=central subfield thickness; SD-OCT=spectral domain optical coherence tomography; CRC=central reading center; SOC=standard of care; mos=months; QID=4 times per day; TID=3 times per day; BID=twice per day; QD=once per day; QOD=once every other day; wk=week; PI=principal investigator.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Definitions

Unless otherwise defined, all technical terms used herein have the same meaning as commonly understood by one of ordinary skill in the art.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value. Reference to “about” a value or parameter herein includes (and describes) embodiments that are directed to that value or parameter per se.

The term “subject”, “patient”, or “individual” refers to primates, such as humans and non-human primates, e.g., African green monkeys and rhesus monkeys. In some embodiments, the subject is a human.

The terms “treat,” “treating”, “treatment,” “ameliorate” or “ameliorating” and other grammatical equivalents as used herein, refer to alleviating, abating or ameliorating an ocular neovascular disease or disorder, or symptoms of the ocular neovascular disease or disorder, preventing additional symptoms of the ocular neovascular disease or disorder, ameliorating or preventing the underlying metabolic causes of symptoms, inhibiting the ocular neovascular disease or disorder, e.g., arresting the development of the ocular neovascular disease or disorder, relieving the ocular neovascular disease or disorder, causing regression of the ocular neovascular disease or disorder, or stopping the symptoms of the ocular neovascular disease or disorder, and are intended to include prophylaxis. The terms further include achieving a therapeutic benefit and/or a prophylactic benefit. The term “therapeutic benefit” refers to eradication or amelioration of the ocular neovascular disease or disorder being treated. Also, a therapeutic benefit is achieved with the eradication or amelioration of one or more of the physiological symptoms associated with the ocular neovascular disease or disorder such that an improvement is observed in the subject, notwithstanding that, in some embodiments, the subject is still afflicted with the ocular neovascular disease or disorder. For prophylactic benefit, the pharmaceutical compositions are administered to a subject at risk of developing the ocular neovascular disease or disorder, or to a subject reporting one or more of the physiological symptoms of the ocular neovascular disease or disorder, even if a diagnosis of the disease or disorder has not been made.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” as used herein, can refer to a sufficient amount of at least one pharmaceutical composition or compound being administered which will relieve to some extent one or more of the symptoms of the ocular disease or disorder being treated. An “effective amount”, “therapeutically effective amount” or “pharmaceutically effective amount” of a pharmaceutical composition may be administered to a subject in need thereof as a unit dose (as described in further detail elsewhere herein).

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

An “AAV vector” or “rAAV vector” as used herein refers to an adeno-associated virus (AAV) vector or a recombinant AAV (rAAV) vector comprising a polynucleotide sequence not of AAV origin (e.g., a polynucleotide heterologous to AAV such as a nucleic acid sequence that encodes a therapeutic transgene, e.g., aflibercept) for transduction into a target cell or to a target tissue. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the at least one AAV capsid protein is from a wild type AAV or is a variant AAV capsid protein (e.g., an AAV capsid protein with an insertion, e.g., an insertion of the 7m8 amino sequence as set forth below). If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus. AAV rep and cap are referred to herein as AAV “packaging genes.”

The term “polypeptide” can encompass both naturally occurring and non-naturally occurring proteins (e.g., a fusion protein), peptides, fragments, mutants, derivatives and analogs thereof. A polypeptide may be monomeric, dimeric, trimeric, or polymeric. Further, a polypeptide may comprise a number of different domains, each of which has one or more distinct activities. For the avoidance of doubt, a “polypeptide” may be any length greater two amino acids.

As used herein, “polypeptide variant” or simply “variant” refers to a polypeptide whose sequence contains an amino acid modification. In some embodiments, the modification is an insertion, duplication, deletion, rearrangement or substitution of one or more amino acids compared to the amino acid sequence of a reference protein or polypeptide, such as a native or wild type protein. A variant may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A variant can have the same or a different biological activity compared to the reference protein, or the unmodified protein.

In some embodiments, a variant can have, for example, at least about 80%, 85%, 90%, 91%, 92%, 93%, 94%, 95%, 96%, 97%, 98%, or 99% overall sequence homology to its counterpart reference protein. In some embodiments, a variant can have at least about 90% overall sequence homology to the wild-type protein. In some embodiments, a variant exhibits at least about 95%, at least about 98%, at least about 99%, at least about 99.5%, or at least about 99.9% overall sequence identity.

As used herein, “recombinant” can refer to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell.

The term “anti-VEGF agent” includes any therapeutic agent, including proteins, polypeptides, peptides, fusion protein, multimeric proteins, gene products, antibody, human monoclonal antibody, antibody fragment, aptamer, small molecule, kinase inhibitor, receptor or receptor fragment, or nucleic acid molecule, that can reduce, interfere with, disrupt, block and/or inhibit the activity or function of an endogenous VEGF and/or an endogenous VEGF receptor (VEGFR), or the VEGF-VEGFR interaction or pathway in vivo. An anti-VEGF agent can be any one of the known therapeutic agents that can reduce new blood vessel growth or formation and/or oedem, or swelling, when delivered into a cell, tissue, or a subject in vivo, e.g., ranibizumab, brolucizumab, or bevacizumab. In some embodiments, an anti-VEGF agent can be naturally occurring, non-naturally occurring, or synthetic. In some embodiments, an anti-VEGF agent can be derived from a naturally occurring molecule that was subsequently modified or mutated to confer an anti-VEGF activity. In some embodiments, an anti-VEGF agent is a fusion or chimeric protein. In such proteins, functional domains or polypeptides are artificially fused to a moiety or a polypeptide to make a fusion or chimeric protein that can sequester VEGF in vivo or function as a VEGFR decoy. In some embodiments, an anti-VEGF agent is a fusion or chimeric protein that blocks endogenous VEGFR from interacting with its ligands.

As used herein, “VEGF” can refer to any isoform of VEGF, unless required otherwise, including, but not limited to, VEGF-A, VEGF-B, VEGF-C, VEGF-D, VEGF-E, VEGF-F, or any combination, or any functional fragment or variant thereof. Unless required otherwise, “VEGF” can refer to any member of the VEGF family, including members: VEGF-A, placenta growth factor (PGF), VEGF-B, VEGF-C, and VEGF-D, or any combination, functional fragment, or variant thereof. As used herein, “VEGF receptor” or “VEGFR” or “VEGF-R” can be used to refer to any one of the receptors of VEGF, including, but not limited to, VEGFR-1 (or Flt-1), VEGFR-2 (or Flk-1/KDR), and VEGFR-3 (or Flt-4). VEGFR can be a membrane bound or soluble form, or a functional fragment or truncation of a receptor. Examples of anti-VEGF agents include, but are not limited to, ranibizumab, bevacizumab, brolucizumab, or any combination, variant, or functional fragment thereof.

“Operatively linked” or “operably linked” or “coupled” can refer to a juxtaposition of genetic elements, wherein the elements are in a relationship permitting them to operate in an expected manner. For instance, a promoter can be operatively linked to a coding region if the promoter helps initiate transcription of the coding sequence. There may be intervening residues between the promoter and coding region so long as this functional relationship is maintained.

The term “expression vector” or “expression construct” or “cassette” or “plasmid” or simply “vector” can include any type of genetic construct, including AAV or rAAV vectors, containing a nucleic acid or polynucleotide coding for a gene product in which part or all of the nucleic acid encoding sequence is capable of being transcribed and is adapted for gene therapy. The transcript can be translated into a protein. In some embodiments, the transcript is partially translated or not translated. In certain aspects, expression includes both transcription of a gene and translation of mRNA into a gene product. In other aspects, expression only includes transcription of the nucleic acid encoding genes of interest. An expression vector can also comprise control elements operatively linked to the encoding region to facilitate expression of the protein in target cells. The combination of control elements and a gene or genes to which they are operably linked for expression can sometimes be referred to as an “expression cassette,” a large number of which are known and available in the art or can be readily constructed from components that are available in the art.

The term “heterologous” can refer to an entity that is genotypically distinct from that of the rest of the entity to which it is being compared. For example, a polynucleotide introduced by genetic engineering techniques into a plasmid or vector derived from a different species can be a heterologous polynucleotide. A promoter removed from its native coding sequence and operatively linked to a coding sequence with which it is not naturally found linked can be a heterologous promoter.

As used herein, “7m8” refers to the amino acid sequence LALGETTRPA (SEQ ID NO: 1).

“7m8 variant” refers to a rAAV, which can be of any serotype, with the amino acid sequence LALGETTRPA (SEQ ID NO: 1) inserted in the solvent exposed GH loop of the capsid protein.

When 7m8 is inserted in a rAAV2 (also referred to as AAV2.7m8), the amino acid sequence LALGETTRPA (SEQ ID NO: 1) is inserted into the GH loop within amino acids 570-611 of the AAV2 capsid protein, e.g., between positions 587 and 588 of the AAV2 capsid protein, VP1. In some cases, when 7m8 is inserted in a rAAV2 (also referred to as AAV2.7m8), the amino acid sequence LALGETTRPA (SEQ ID NO: 1) is inserted into the GH loop of the AAV2 capsid protein, e.g., between positions 587 and 588 of AAV2 VP1 comprising the sequence of SEQ ID NO: 13. When 7m8 is inserted in a rAAV1 (also referred to as AAV1.7m8), the amino acid sequence LALGETTRPA (SEQ ID NO: 1) is inserted into the GH loop within amino acids 571-612 of the AAV1 capsid protein, e.g., between amino acids 590 and 591 of the AAV1 capsid protein. When 7m8 is inserted in a rAAV5 (also referred to as AAV5.7m8), the amino acid sequence LALGETTRPA (SEQ ID NO: 1) is inserted into the GH loop within amino acids 560-601 of the AAV5 capsid protein, e.g., between amino acids 575 and 576 of the AAV5 capsid protein. When 7m8 is inserted in a rAAV6 (also referred to as AAV6.7m8), the amino acid sequence LALGETTRPA (SEQ ID NO: 1) is inserted into the GH loop within amino acids 571 to 612 of the AAV6 capsid protein, e.g., between amino acids 590 and 591 of the AAV6 capsid protein. When 7m8 is inserted in a rAAV7 (also referred to as AAV7.7m8), the amino acid sequence LALGETTRPA (SEQ ID NO: 1) is inserted into the GH loop within amino acids 572 to 613 of the AAV7 capsid protein, e.g., between amino acids 589 and 590 of the AAV7 capsid protein. When 7m8 is inserted in a rAAV8 (also referred to as AAV8.7m8), the amino acid sequence LALGETTRPA (SEQ ID NO: 1) is inserted into the GH loop within amino acids 573 to 614 of the AAV8 capsid protein, e.g., between amino acids 590 and 591 of the AAV8 capsid protein. When 7m8 is inserted in a rAAV9 (also referred to as AAV9.7m8), the amino acid sequence LALGETTRPA (SEQ ID NO: 1) is inserted into the GH loop of the AAV9 capsid protein, e.g., between amino acids 588 and 589 of the AAV9 capsid protein. When 7m8 is inserted in a rAAV10 (also referred to as AAV10.7m8), the amino acid sequence LALGETTRPA (SEQ ID NO: 1) is inserted into the GH loop within amino acids 573 to 614 of the AAV10 capsid protein, e.g., between amino acids 589 and 590 of the AAV10 capsid protein.

### Overview

Current therapies (e.g., aflibercept recombinant protein, ranibizumab recombinant protein) for ocular neovascular diseases such as wAMD require lifelong IVT administration approximately every 4-8 weeks. This can increase the risk of inflammation, infection, and other adverse effects in some patients. Further, current therapies create compliance challenges due to repeated and/or frequent trips to medical offices for administration of the therapy, especially in elderly patients, who are most affected with wAMD. Reduction in frequency of administration is associated with vision loss and deterioration of the eye disease or condition. The ability of AAV vectors to efficiently transduce target retinal cells following IVT injection has been exploited to successfully transfer therapeutic genes into photoreceptors, retinal pigment epithelium, and the inner retina to treat a variety of retinal diseases. Thus, administration of rAAV particles encoding an anti-VEGF agent (e.g., aflibercept) can provide prolonged and/or sustained release of the anti-VEGF agent in vivo.

Surprisingly, administration of a single low unit dose of 6×10¹¹ vector genomes (vg) per eye of rAAV particles encoding aflibercept to the eyes of individuals with an ocular neovascular disease led to stabilization of the disease and a robust anatomical response in all treated individuals (See Example 1). In addition, visual acuity was stabilized in all treated individuals and none of the individuals required rescue anti-VEGF treatment (e.g., aflibercept IVT injections) after administration of the single low unit dose of 6×10¹¹ vg/eye of rAAV particles encoding aflibercept. Moreover, administration of the single unit dose of rAAV particles encoding aflibercept to the eyes of individuals with an ocular neovascular disease unexpectedly caused a reduction (e.g., resolution) of symptoms, including intraretinal and subretinal fluid that were refractory to prior anti-VEGF treatments (e.g., chronic IVT injections of aflibercept, ranibizumab, or bevacizumab).

Accordingly, the present disclosure provides methods of treating an ocular neovascular disease in an individual by administering a single unit dose of 6×10¹¹ vg/eye or less of rAAV particles encoding an anti-VEGF agent (e.g., aflibercept). In addition, the present disclosure provides methods for reducing retinal fluid in the eye of an individual with an ocular neovascular disease by administering a single unit dose of rAAV particles encoding an anti-VEGF agent (e.g., aflibercept). The methods disclosed herein reduce or eliminate the need for repeated IVT injections while providing long-term efficacy, thereby addressing the non-compliance and non-adherence problem. In addition, the methods provided herein reduce the adverse effects associated with multiple IVT injections.

### Methods of Treatment

Provided herein is a method for treating an ocular neovascular disease in an individual, the method comprising administering a unit dose of recombinant adeno-associated virus (rAAV) particles to an eye of the individual.

Also provided herein is a method for reducing retinal fluid in the eye of an individual with an ocular neovascular disease, the method comprising administering a unit dose of rAAV particles to an eye of the individual.

Also provided herein is a method for treating an ocular neovascular disease in an individual, the method comprising administering an anti-VEGF agent (e.g., aflibercept) to an eye of the individual, and administering a unit dose of recombinant adeno-associated virus (rAAV) particles to the eye of the individual after administration of the anti-VEGF agent.

In some embodiments, the ocular neovascular disease is wet age-related macular degeneration (wAMD), retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy, diabetic retinal edema, or any combination thereof.

In some embodiments, the term ocular neovascular disease also encompasses VEGF-driven pre-neovascular diseases which, if left untreated, progress to a neovascular form. In some embodiments, the ocular neovascular disease is the pre-neovascular disease, non-proliferative diabetic retinopathy.

In some embodiments, the individual is a human. In some embodiments, the individual received at least one prior treatment (e.g., at least one, at least two, at least three, at least four, at least 5 or more treatments) for the ocular neovascular disease with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in about the last 8 weeks, about the last 9 weeks, about the last 10 weeks, about the last 11 weeks, about the last 12 weeks, about the last 13 weeks, about the last 14 weeks, about the last 15 weeks, or about the last 16 weeks prior to administration of the unit dose of rAAV particles. In some embodiments, the individual demonstrated a meaningful response to a prior treatment with anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the anti-VEGF agent is aflibercept, a functional variant thereof, or a functional fragment thereof. In some embodiments, the anti-VEGF agent comprises a polypeptide comprising an amino acid sequence with at least about 95% identity to the amino acid sequence of SEQ ID NO: 35. In some embodiments, the retinal fluid in the eye of an individual is intraretinal fluid (IRF) and/or subretinal fluid (SRF). In some embodiments, the amount or presence of retinal fluid in the eye of the individual is refractory to prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the anti-VEGF agent is aflibercept, a functional variant thereof, or a functional fragment thereof. In some embodiments, the anti-VEGF agent comprises a polypeptide comprising an amino acid sequence with at least about 95% identity to the amino acid sequence of SEQ ID NO: 35.

In some embodiments, the ocular neovascular disease is diabetic macular edema (DME). In some embodiments, the individual is a human. In some embodiments, the individual has type 1 or type 2 diabetes mellitus. In some embodiments, the individual has vision impairment that is due to center involving diabetic macular edema. In some embodiments, the individual has visual acuity (BCVA) of between about 78 to 50 ETDRS letters (e.g., any of 50, 51, 52, 53 54, 55, 56, 57, 58, 59, 60, 61, 62, 63, 64, 65, 66, 67, 68, 69, 70, 71, 72, 73, 74, 75, 76, 77, or 78 ETDRS letters) in the eye administered the rAAV particles prior to administration of the unit dose of rAAV particles. In some embodiments, the individual has visual acuity (Snellen equivalent) of between about 20/32 to about 20/100 in the eye administered the rAAV particles prior to administration of the unit dose of rAAV particles. In some embodiments, the individual has a central subfield thickness (CST) of ≥325 μm using Heidelberg Spectralis® with center-involving IRF (center 1 mm) in the eye administered the rAAV particles prior to administration of the unit dose of rAAV particles. In some embodiments, the individual has a decrease in vision in the eye administered the rAAV particles prior to administration of the unit dose of rAAV particles that is primarily due to diabetic macular edema. In some embodiments, the individual was diagnosed with diabetic macular edema in the eye administered the rAAV particles about 6 months or less prior to administration of the unit dose of rAAV particles, e.g., any of about 6 months, about 5 months, about 4 months, about 3 months, about 2 months, about 1 month, or less, prior to administration of the unit dose of rAAV particles. In some embodiments, the individual received 0, 1, or 2 prior treatments for DME in the eye administered the rAAV particles, e.g., 0, 1, or 2 intravitreal injections with an anti-VEGF agent, e.g., aflibercept, prior to administration of the unit dose of rAAV particles. In some embodiments, the individual received the prior treatments with an anti-VEGF agent in the eye administered the rAAV particles at least about 60 days (i.e., about 2 months) prior to administration of the unit dose of rAAV particles. In some embodiments, the individual exhibited a meaningful response in central subfield thickness to the prior treatments with an anti-VEGF agent in the eye administered the rAAV particles prior to administration of the unit dose of rAAV particles, for example, at least a 10% reduction in central subfield thickness. In some embodiments, the individual did not experience an adverse reaction to the prior treatments with an anti-VEGF agent prior to administration of the unit dose of rAAV particles. In some embodiments, the individual does not have neutralizing antibodies to AAV2.7m8 prior to administration of the unit dose of rAAV particles. In some embodiments, the individual does not have an anti-AAV2.7m8 neutralizing antibody titer of greater than 1:125 prior to administration of the unit dose of rAAV particles, e.g., within about 6 months prior to administration of the unit dose of rAAV particles. In some embodiments, the individual does not have a history of allergy to aflibercept, corticosteroids, or fluorescein dye or sodium fluorescein (e.g., used in angiography) prior to administration of the unit dose of rAAV particles. In some embodiments, the individual has a history of mild allergy to aflibercept, corticosteroid, or fluorescein dye or sodium fluorescein (e.g., used in angiography) prior to administration of the unit dose of rAAV particles, wherein the allergy is amenable to treatment. In some embodiments, the individual does not have uncontrolled diabetes, e.g., HbA1C of greater than 10%, prior to administration of the unit dose of rAAV particles. In some embodiments, the individual does not have a history of diabetic ketoacidosis within about 3 months prior to administration of the unit dose of rAAV particles. In some embodiments, the individual has not initiated intensive insulin treatment, e.g., with an insulin pump or multiple daily insulin injections, prior to administration of the unit dose of rAAV particles. In some embodiments, the individual does not plan to initiate intensive insulin treatment, e.g., with an insulin pump or multiple daily insulin injections, within about 3 months after administration of the unit dose of rAAV particles. In some embodiments, the individual does not have a history of systemic autoimmune disease that requires treatment with systemic steroids or immunosuppressive treatments, e.g., methotrexate or adalimumab, prior to administration of the unit dose of rAAV particles. In some embodiments, the individual is not being administered a systemic drug known to cause macular edema, such as fingolimod, tamoxifen, chloroquine, or hydroxychloroquine, prior to administration of the unit dose of rAAV particles. In some embodiments, the individual is not being administered a systemic anti-VEGF treatment prior to administration of the unit dose of rAAV particles. In some embodiments, the individual does not have high-risk proliferative diabetic retinopathy (PDR) in the eye administered the rAAV particles prior to administration of the unit dose of rAAV particles. In some embodiments, PDR is defined as any vitreous or preretinal hemorrhage, neovascularization elsewhere >½-disc area within an area equivalent to standard ETDRS 7-field on clinical examination, or neovascularization of disc>⅓-disc area on clinical examination. In some embodiments, the individual does not have focal or grid laser photocoagulation in the eye administered the rAAV particles prior to administration of the unit dose of rAAV particles. In some embodiments, the individual does not have any prior pan retinal photocoagulation (PRP) in the eye administered the rAAV particles prior to administration of the unit dose of rAAV particles. In some embodiments, the individual has not received an anti-VEGF therapy (e.g., aflibercept IVT injections) in the eye administered the rAAV particles prior to administration of the unit dose of rAAV particles. In some embodiments, the individual has not received an anti-VEGF therapy (e.g., aflibercept IVT injections) in the eye administered the rAAV particles for at least 60 days prior to administration of the unit dose of rAAV particles. In some embodiments, the individual has not received more than two anti-VEGF treatments (e.g., aflibercept IVT injections) in the eye administered the rAAV particles prior to administration of the unit dose of rAAV particles. In some embodiments, the individual does not have a history of any of anterior segment neovascularization (e.g., neovascularization of the iris [NVI] or neovascular glaucoma [NVG]), significant vitreous hemorrhage, fibrovascular proliferation, or tractional retinal detachment in the eye administered the rAAV particles prior to administration of the unit dose of rAAV particles. In some embodiments, the individual does not have structural abnormalities at the fovea (e.g., any of dense hard exudates, pigment abnormalities, foveal atrophy, vitreomacular traction or epiretinal membrane) in the eye administered the rAAV particles that contribute to macular edema or visual impairment prior to administration of the unit dose of rAAV particles. In some embodiments, structural abnormalities at the fovea are assessed on clinical examination or OCT. In some embodiments, the individual does not have a history of retinal disease other than diabetic retinopathy (e.g., age-related macular degeneration (in either eye), retinal vein occlusion, retinal arterial occlusion, or pathologic myopia) in the eye administered the rAAV particles prior to administration of the unit dose of rAAV particles. In some embodiments, the individual does not have history of ocular disease other than diabetic macular edema in the eye administered the rAAV particles, e.g., a significant cataract or macular traction, or evidence of posterior subcapsular cataract, prior to administration of the unit dose of rAAV particles. In some embodiments, the individual does not have history of cataract extraction or Yttrium Aluminum Garnet (YAG) capsulotomy in the eye administered the rAAV particles within at least about 3 months prior to administration of the unit dose of rAAV particles. In some embodiments, the individual does not have a history of retinal detachment (with or without repair) in the eye administered the rAAV particles prior to administration of the unit dose of rAAV particles. In some embodiments, the individual does not have a history of any of trabeculectomy, glaucoma shunt, or minimally invasive glaucoma surgery (MIGS) in the eye administered the rAAV particles prior to administration of the unit dose of rAAV particles. In some embodiments, the individual does not have a history of vitrectomy or other filtration surgery in the eye administered the rAAV particles prior to administration of the unit dose of rAAV particles. In some embodiments, the individual does not have aphakia or presence of an anterior chamber intraocular lens in the eye administered the rAAV particles prior to administration of the unit dose of rAAV particles. In some embodiments, the individual does not have uncontrolled ocular hypertension or glaucoma in the eye administered the rAAV particles, e.g., IOP>22 mmHg despite treatment with anti-glaucoma medication or current use of >2 IOP lowering medications, prior to administration of the unit dose of rAAV particles. In some embodiments, the individual does not have a history of intraocular or periocular steroid treatment for any ocular condition (e.g., IVT Triesence, Iluvien or Ozurdex) in the eye administered the rAAV particles prior to administration of the unit dose of rAAV particles. In some embodiments, the individual has not had refractive surgery in the eye administered the rAAV particles within at least about 90 days prior to administration of the unit dose of rAAV particles. In some embodiments, the individual does not have previous penetrating keratoplasty, endothelial keratoplasty, or ocular radiation in the eye administered the rAAV particles prior to administration of the unit dose of rAAV particles. In some embodiments, the individual has not had any prior vitreoretinal surgery in the eye administered the rAAV particles prior to administration of the unit dose of rAAV particles. In some embodiments, the individual does not have a history of uveitis or intraocular inflammation, e.g., grade trace or above except mild anticipated post-operative inflammation that resolved, prior to administration of the unit dose of rAAV particles. In some embodiments, the individual does not have a history of IOP elevation that is related to topical steroid administration prior to administration of the unit dose of rAAV particles. In some embodiments, the individual does not have a history of ocular Herpes Simplex Virus (HSV), Varicella-zoster virus (VZV), or Cytomegalovirus (CMV), including viral uveitis, retinitis or keratitis prior to administration of the unit dose of rAAV particles. In some embodiments, the individual does not have evidence of any of external ocular infection, including conjunctivitis, chalazion, or significant blepharitis prior to administration of the unit dose of rAAV particles. In some embodiments, the individual does not have history of ocular toxoplasmosis prior to administration of the unit dose of rAAV particles.

In some embodiments, the unit dose is expressed as the number of vector genomes (vg). In some embodiments, the unit dose is about 6×10¹¹ vector genomes (vg) or less of the rAAV particles. In some embodiments, the unit dose is expressed as the number of vector genomes (vg) per eye (vg/eye). In some embodiments, the unit dose is about 6×10¹¹ vg/eye or less of the rAAV particles, e.g., 1×10¹¹ vg/eye. In some embodiments, the unit dose of rAAV particles is about 6×10¹⁰ to about 2×10¹¹ vg/eye. In some embodiments the unit dose is 3×10¹¹ vg/eye. In some embodiments, the unit dose of rAAV particles is about 2×10¹¹ or about 6×10¹⁰ vg/eye.

In some embodiments, the unit dose of rAAV particles is administered to one eye of the individual. In some embodiments, the one eye of the individual is the right eye or the left eye. In some embodiments, the one eye of the individual is the right eye. In some embodiments, the one eye of the individual is the left eye. In some embodiments, the methods provided herein further comprise administering a unit dose of rAAV particles to the contralateral eye of the individual. In some embodiments, the one eye of the individual is the right eye and the contralateral eye is the left eye. In some embodiments, the one eye of the individual is the left eye and the contralateral eye is the right eye.

In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye. In some embodiments, the unit dose of rAAV particles administered to the contralateral eye of the individual is about the same as (e.g., less than 1% higher or lower, less than 5% higher or lower, less than 10% higher or lower, or less than 20% higher or lower) or lower (e.g., about 5%, about 10%, about 20%, about 30%, about 40%, about 50%, about 60%, about 70%, about 80%, or about 90% lower) than the unit dose of rAAV particles administered to the one eye of the individual.

In some embodiments, the administering the unit dose of rAAV particles to the contralateral eye is at least about 2 weeks (e.g., at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, or more) after administering the unit dose of rAAV particles to the one eye. In some embodiments, the unit dose of rAAV particles administered to the contralateral eye of the individual is higher (e.g., any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, higher) than the unit dose of rAAV particles administered to the one eye of the individual.

In some embodiments, the rAAV particles comprise a) a nucleic acid encoding a polypeptide comprising an amino acid sequence with at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein. The sequence of SEQ ID NO: 35 is provided below:

(SEQ ID NO: 35)
SDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQ
HKKLVNRDLKTQSGSEMKKFLSTLTIDGVTRSDQGLYTCAASSGLMTKKNSTFVRVHEKDKTHTCPPCPAPEL
LGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPG

In some embodiments, the rAAV particles comprise a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

In some embodiments, the rAAV particles comprise a nucleic acid encoding a polypeptide comprising an amino acid sequence with at least about 80%, at least about 85%, at least about 90%, at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the polypeptide comprises the amino acid sequence of SEQ ID NO: 35. In some embodiments, the polypeptide is aflibercept or a functional variant thereof or functional fragment thereof.

In some embodiments, the rAAV particles comprise a nucleic acid comprising a codon-optimized sequence encoding an amino acid sequence with at least about 80%, at least about 85%, at least about 90%, at least about 95%, at least about 96%, at least about 97%, at least about 98%, at least about 99%, at least about 99.99%, or 100% identity to the amino acid sequence of SEQ ID NO: 35 and flanked by AAV2 inverted terminal repeats (ITRs). In some embodiments, the rAAV particles comprise a nucleic acid comprising a codon-optimized sequence encoding an amino acid sequence with at least about 95% identity to the amino acid sequence of SEQ ID NO: 35 and flanked by AAV2 inverted terminal repeats (ITRs). In some embodiments, the rAAV particles comprise a nucleic acid comprising a codon-optimized sequence encoding an amino acid sequence with 100% identity to the amino acid sequence of SEQ ID NO: 35 and flanked by AAV2 inverted terminal repeats (ITRs).

In some embodiments, the rAAV particles comprise a nucleic acid comprising the cDNA sequence of aflibercept or a functional variant thereof or functional fragment thereof and flanked by AAV2 inverted terminal repeats (ITRs). In some embodiments, the rAAV particles comprise a nucleic acid comprising a codon-optimized cDNA sequence of aflibercept or a functional variant thereof or functional fragment thereof and flanked by AAV2 inverted terminal repeats (ITRs). In some embodiments, the rAAV particles comprise a nucleic acid comprising the nucleic acid sequence of SEQ ID NO: 36.

In some embodiments, the nucleic acid further comprises (a) a first enhancer region comprising a CMV sequence; (b) a promoter region comprising a CMV sequence; (c) a 5′UTR region comprising, in the 5′ to 3′ order, a TPL sequence and an eMLP sequence; (d) a second enhancer region comprising a full EES sequence; and (e) a HGH polyadenylation site. In some embodiments, the enhancer region comprising a CMV sequence comprises the sequence of SEQ ID NO: 22. In some embodiments, the promoter region comprising a CMV sequence comprises the sequence of SEQ ID NO: 23. In some embodiments, the TPL sequence comprises the sequence of SEQ ID NO: 24. In some embodiments, the eMLP sequence comprises the sequence of SEQ ID NO: 25. In some embodiments, the second enhancer region comprising a full EES sequence comprises the sequence of SEQ ID NO: 26. In some embodiments, the HGH polyadenylation site comprises the sequence of SEQ ID NO: 27.

In some embodiments, the rAAV particles comprise an AAV2 capsid protein comprising the amino acid sequence LGETTRP (SEQ ID NO: 14) inserted between positions 587 and 588 of the AAV2 VP1 comprising the sequence of SEQ ID NO: 13. The sequence of SEQ ID NO: 13 is provided below:

(SEQ ID NO: 13)
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGAD
GVGNSSGNWHCDSTWMGDRVITTSTRTWALPTYNNHLYKQISSQSGASNDNHYFGYSTPWGYFDFNRFHCHFS
PRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLI
DQYLYYLSRTNTPSGTTTQSRLQFSQAGASDIRDQSRNWLPGPCYRQQRVSKTSADNNNSEYSWTGATKYHLN
GRDSLVNPGPAMASHKDDEEKFFPQSGVLIFGKQGSEKTNVDIEKVMITDEEEIRTTNPVATEQYGSVSTNLQ
RGNRQAATADVNTQGVLPGMVWQDRDVYLQGPIWAKIPHTDGHFHPSPLMGGFGLKHPPPQILIKNTPVPANP
STTFSAAKFASFITQYSTGQVSVEIEWELQKENSKRWNPEIQYTSNYNKSVNVDFTVDINGVYSEPRPIGTRY
LTRNL

In some embodiments, the rAAV particles comprise an AAV2 capsid protein comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise an AAV2 capsid protein comprising the amino acid sequence LALGETTRPA (SEQ ID NO: 1) inserted between positions 587 and 588 of the AAV2 VP1 comprising the sequence of SEQ ID NO: 13.

In some embodiments, the rAAV particles comprise an AAV2 capsid protein comprising any of the following amino acid sequences inserted between positions 587 and 588 of the capsid protein, wherein the amino acid residue numbering corresponds to an AAV2 VP1 capsid protein: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21). In some embodiments, the rAAV particles comprise an AAV2 capsid protein comprising any of the following amino acid sequences inserted between positions 587 and 588 of the AAV2 VP1 comprising the sequence of SEQ ID NO: 13: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21).

In some embodiments, the administration of the unit dose of rAAV particles to the one eye and/or to the contralateral eye of the individual is by intravitreal (IVT) injection, intraocular administration, or intraretinal injection. In some embodiments, the administration of the unit dose of rAAV particles to the one eye and/or to the contralateral eye of the individual is by intravitreal (IVT) injection.

In some embodiments, the unit dose of rAAV particles is in a pharmaceutical formulation. In some embodiments, the pharmaceutical formulation comprises the rAAV particles, one or more osmotic or ionic strength agents, one or more buffering agents, one or more surfactants, and one or more solvents. In some embodiments, the osmotic or ionic strength agent is sodium chloride. In some embodiments, the one or more buffering agents are sodium phosphate monobasic and/or sodium phosphate dibasic. In some embodiments, the surfactant is Poloxamer 188. In some embodiments, the solvent is water. In some embodiments, the pharmaceutical formulation comprises the rAAV particles, sodium chloride, sodium phosphate and a surfactant. In some embodiments, the pharmaceutical formulation comprises about 1×10¹⁰ vg/mL to about 1×10¹³ vg/mL of rAAV particles. In some embodiments, the pharmaceutical formulation comprises about 6×10¹¹ vg/mL to about 6×10¹² vg/mL of rAAV particles. In some embodiments, the pharmaceutical formulation comprises about 150 mM to about 200 mM sodium chloride (e.g., any of about 150 mM, about 160 mM, about 170 mM, about 180 mM, about 190 mM, or about 200 mM). In some embodiments, the pharmaceutical formulation comprises about 1 mM to about 10 mM monobasic sodium phosphate (e.g., about 1 mM, about 2 mM, about 3 mM, about 4 mM, about 5 mM, about 6 mM, about 7 mM, about 8 mM, about 9 mM, or about 10 mM). In some embodiments, the pharmaceutical formulation comprises about 1 mM to about 10 mM dibasic sodium phosphate (e.g., about 1 mM, about 2 mM, about 3 mM, about 4 mM, about 5 mM, about 6 mM, about 7 mM, about 8 mM, about 9 mM, or about 10 mM). In some embodiments, the pharmaceutical formulation comprises about 0.0005% (w/v) to about 0.005% (w/v) poloxamer 188 (e.g., any of about 0.0005% (w/v), 0.0006% (w/v), 0.0007% (w/v), 0.0008% (w/v), 0.0009% (w/v), 0.001% (w/v), 0.002% (w/v), 0.003% (w/v), 0.004% (w/v), or about 0.005% (w/v)). In some embodiments, the pharmaceutical formulation has a pH of about 7.0 to about 7.5 (e.g., any of about 7.0, about 7.1, about 7.2, about 7.3, about 7.4, or about 7.5). In some embodiments, the pharmaceutical formulation comprises about 6×10¹² vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3. In some embodiments, the pharmaceutical formulation comprises about 6×10¹¹ vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3.

In some embodiments, the unit dose of rAAV particles comprises a volume of between about 25 μL to about 250 μL (e.g., any of about 25 μL, about 30 μL, about 40 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, or about 250 μL). In some embodiments, the concentration of rAAV particles in the pharmaceutical formulation is adjusted such that the volume of the unit dose of rAAV particles administered to an eye of the individual is between about 25 μL to about 250 μL. In some embodiments, the unit dose of rAAV particles comprises a volume of about 100 μL. In some embodiments, the unit dose of rAAV particles comprises a volume of about 30 μL.

In some embodiments, the unit dose of rAAV particles is administered in combination with steroid treatment. In some embodiments, the steroid treatment is a corticosteroid treatment. In some embodiments, the steroid treatment is a systemic steroid treatment. In some embodiments, the steroid treatment is an oral steroid treatment. In some embodiments, the steroid treatment is a prednisone treatment. In some embodiments, the steroid treatment is an ophthalmic steroid treatment. In some embodiments, the ophthalmic steroid treatment is a topical steroid treatment (e.g., a drop), a periocular steroid treatment (e.g., subtenons, subconjunctival), an intravitreal steroid treatment, or a superchoroidal steroid treatment. In some embodiments, the ophthalmic steroid treatment is a glucocorticoid including, but not limited to, an anti-inflammatory glucocorticoid. In some embodiments, the topical steroid treatment is a glucocorticoid including but not limited to, an anti-inflammatory glucocorticoid. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. Anti-inflammatory glucocorticoids may include, but are not limited to, difluprednate, dexamethasone, prednisolone, triamcinolone, fluorometholone, rimexolone, fluocinolone, loteprednol and bioequivalents thereof. In some embodiments, the topical steroid treatment is a difluprednate treatment. By “dexamethasone” is intended dexamethasone, dexamethasone biosimilars, dexamethasone bioequivalents, and pharmaceutical compositions comprising dexamethasone, a dexamethasone biosimilar or a dexamethasone bioequivalent. Pharmaceutical compositions comprising dexamethasone include, but are not limited to, Ozurdex™, Maxidex™, Decadron™, Dexamethasone Intensol™, Ocu-Dex™, Dexycu™, Dextenza™ and Zodex™. Ozurdex™ is a pharmaceutical composition comprising dexamethasone. By “difluprednate” is intended difluprednate, difluprednate biosimilars, difluprednate bioequivalents, and pharmaceutical compositions comprising difluprednate, a difluprednate biosimilar or a difluprednate bioequivalent. Pharmaceutical compositions comprising difluprednate include, but are not limited to, Durezol™ and difluprednate emulsions. By “triamcinolone” is intended triamcinolone, triamcinolone biosimilars, triamcinolone bioequivalents, and pharmaceutical compositions comprising triamcinolone, a triamcinolone biosimilar or a triamcinolone bioequivalent. Pharmaceutical compositions comprising triamcinolone include, but are not limited to, Triesence™, Xipere™, and Trivaris™. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment (e.g., difluprednate). In some embodiments, the ophthalmic steroid treatment (e.g., difluprednate) is a daily steroid treatment for up to about 4 weeks, about 6 weeks, or about 8 weeks from administering the unit dose of rAAV particles. In some embodiments, the ophthalmic steroid treatment comprises about four administrations of ophthalmic steroid on about week 1, about three administrations of ophthalmic steroid on about week 2, about two administrations of ophthalmic steroid on about week 3, and about one administration of ophthalmic steroid on about week 4; timing starting with and following administration of the unit dose of rAAV particles. In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the steroid treatment is an ophthalmic steroid treatment (e.g., difluprednate). In some embodiments, the ophthalmic steroid treatment (e.g., difluprednate) is a daily topical steroid treatment for up to about 4 weeks, about 6 weeks, or about 8 weeks from administering the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid (i.e., QID) per day for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the topical steroid comprises difluprednate 0.05% at a dose of about 1 μg to about 3 μg. In some embodiments, the topical steroid comprises difluprednate 0.05% at a dose of about 2.5 μg. In some embodiments, the topical steroid is about 0.005% to about 0.5% difluprednate. In some embodiments, the topical steroid is any of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, or about 0.1% difluprednate. In some embodiments, the topical steroid is difluprednate 0.05%. In some embodiments, a dose of difluprednate 0.05% is one drop of ophthalmic solution. In some embodiments, one drop is about 50 μl (e.g., about 25 μl to about 50 μl, about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some aspects, the invention provides methods for treating an ocular neovascular disease in an individual, the method comprising administering of recombinant adeno-associated virus (rAAV) particles to one eye of the individual, wherein the individual is a human, wherein the rAAV particles comprise: a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein, and wherein the unit dose of rAAV particles is administered in combination with an oral steroid treatment and a topical steroid treatment or an oral steroid treatment and an intravitreal (IVT) steroid treatment. In some embodiments, the oral steroid treatment and the topical steroid treatment or the oral steroid treatment and the IVT are a corticosteroid treatments. In some embodiments, the topical steroid treatment is a difluprednate treatment. In some embodiments, the topical steroid treatment comprises difluprednate 0.05% at a dose of about 1 μg to about 3 μg. In some embodiments, the topical steroid treatment comprises difluprednate 0.05% at a dose of about 2.5 μg. In some embodiments, the topical steroid treatment comprises about four administrations of topical steroid per day on about day 7 prior to administration of the unit dose of rAAV particles to about day 35 after administration of the unit does of rAAV particles, about three administrations of topical steroid per day on about day 36 to about day 63 after administration of the unit dose of rAAV particles, about two administrations of topical steroid per day on about day 64 to about day 91 after administration of the unit dose of rAAV particles, about one administrations of topical steroid per day on about day 92 to about day 119 after administration of the unit dose of rAAV particles, and about one administrations of topical steroid every other day starting on about day 120 to about day 147 after administration of the unit dose of rAAV particles. In some embodiments, the topical steroid treatment comprises about four administrations of topical steroid per day about one week prior to administration of the unit dose of rAAV particles to about five weeks after administration of the unit does of rAAV particles, about three administrations of topical steroid per day about 6 weeks to about 9 weeks after administration of the unit dose of rAAV particles, about two administrations of topical steroid per day about 10 weeks to about 13 weeks after administration of the unit dose of rAAV particles, about one administrations of topical steroid per day about 14 weeks to about 17 weeks after administration of the unit dose of rAAV particles, and about one administrations of topical steroid every other day about 18 to about 21 weeks after administration of the unit dose of rAAV particles. In some embodiments, the oral steroid treatment is a prednisone treatment. In some embodiments, the oral steroid treatment is administered at a dose of: less than about 0.8 mg/kg/day to about 1.00 mg/kg/day for the week prior to through week two following administering the unit dose of rAAV particles, less than about 0.59 mg/kg/day to about 0.75 mg/kg/day on weeks three to five following administering the unit dose of rAAV particles, less than about 0.4 mg/kg/day to about 0.6 mg/kg/day on week six after administering the unit dose of rAAV particles, less than about 0.25 mg/kg/day to about 0.375 mg/kg/day on week seven after administering the unit dose of rAAV particles, less than about 0.14 mg/kg/day to about 0.28 mg/kg/day on week eight after administering the unit dose of rAAV particles, and less than about 0.063 mg/kg/day to about 0.125 mg/kg/day on week nine after administering the unit dose of rAAV particles. In some embodiments, the oral steroid treatment is administered at a dose of: less than or equal to about 1.00 mg/kg/day for the week prior to through week two following administering the unit dose of rAAV particles, less than or equal to about 0.75 mg/kg/day on weeks three to five following administering the unit dose of rAAV particles, less than or equal to about 0.6 mg/kg/day on week six after administering the unit dose of rAAV particles, less than or equal to about 0.375 mg/kg/day on week seven after administering the unit dose of rAAV particles, less than or equal to about 0.28 mg/kg/day on week eight after administering the unit dose of rAAV particles, and less than or equal to about 0.125 mg/kg/day on week nine after administering the unit dose of rAAV particles. In some embodiments, the oral steroid treatment is administered at a dose of about 0.125 mg/kg/day to about 0.14 mg/kg/day on the first four days of week nine after administering the unit dose of rAAV particles and about 0.0625 mg/kg/day to about 0.070 mg/kg/day on the first four days of week nine after administering the unit dose of rAAV particles if the individual weighs 70 kg or more. In some embodiments, the IVT steroid treatment is a triamcinolone acetonide treatment, a dexamethasone treatment. In some embodiments, the IVT steroid treatment is a treatment such as, but not limited to, Ozurdex®, Iluvien® or Triesence®. In some embodiments, the IVT steroid treatment is a triamcinolone acetonide treatment of about 2 mg. In some embodiments, the IVT steroid treatment comprises administration of the IVT steroid treatment one week prior to administration of the unit dose of rAAV particles and at week 12 after administration of the unit dose of rAAV particles. In some embodiments, the IVT steroid treatment is a dexamethasone of about 0.7 mg. In some embodiments, the IVT steroid treatment is an intravitreal implant one week prior to administration of the unit does of rAAV particles. In some embodiments, the oral steroid treatment and/or the topical steroid treatment is administered before, during and/or after administration of the unit dose of rAAV particles.

In some aspects, the invention provides a unit dose of recombinant adeno-associated virus (rAAV) particles for use in a method for treating an ocular neovascular disease in an individual, the method comprising administering said unit dose to one eye of the individual, wherein the individual is a human, wherein the rAAV particles comprise a) a nucleic acid encoding a polypeptide comprising an amino acid sequence with at least about 95% identity to the amino acid sequence of SEQ ID NO: 35 and flanked by AAV2 inverted terminal repeats (ITRs), and b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein, and wherein the unit dose of rAAV particles is administered in combination with an oral steroid treatment and a topical steroid treatment or an oral steroid treatment and an intravitreal (IVT) steroid treatment.

In some embodiments, the retinal fluid in the eye of the individual (e.g., SRF and/or IRF) is reduced by more than any of about 5%, about 10%, about 15%, about 20%, about 30%, about 40%, about 50%, about 60%, about 70%, about 80%, about 90%, or about 100% after administration of the unit dose of rAAV particles to the one eye and/or to the contralateral of the individual. In some embodiments, the retinal fluid in the eye of the individual (e.g., SRF and/or IRF) is reduced by more than any of about 5%, about 10%, about 15%, about 20%, about 30%, about 40%, about 50%, about 60%, about 70%, about 80%, about 90%, or about 100% after administration of the unit dose of rAAV particles to the one eye and/or to the contralateral of the individual compared to the level of retinal fluid in the eye of the individual prior to administration of the unit dose or rAAV particles. In some embodiments, the retinal fluid in the eye of the individual (e.g., SRF and/or IRF) is reduced by about 100% after administration of the unit dose of rAAV particles to the one eye and/or to the contralateral of the individual compared to the level of retinal fluid in the eye of the individual prior to administration of the unit dose or rAAV particles.

In some embodiments, the methods provided herein further comprise monitoring the level of retinal fluid (e.g., SRF and/or IRF) in the one eye and/or the contralateral eye of the individual after administration of the unit dose of rAAV particles. In some embodiments, the reduction of retinal fluid (e.g., SRF and/or IRF) in the eye is first observed any of about 1 day, about 3 days, about 8 days, about 2 weeks, about 4 weeks, about 6 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, about 104 weeks, or more after administration of the unit dose of rAAV particles. In some embodiments, the reduction of retinal fluid (e.g., SRF and/or IRF) in the eye continues or is maintained for at least 1 week, at least 2 weeks, at least 4 weeks, at least 6 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, at least 72 weeks, at least 76 weeks, at least 80 weeks, at least 84 weeks, at least 88 weeks, at least 92 weeks, at least 96 weeks, at least 100 weeks, at least 104 weeks, or more after administration of the unit dose of rAAV particles.

In some embodiments, the reduction of retinal fluid (e.g., SRF and/or IRF) in the eye is determined by any method known in the art. In some embodiments, the reduction of retinal fluid (e.g., SRF and/or IRF) in the eye is determined by optical coherence tomography (OCT), spectral domain OCT (SD-OCT), OCT angiography, fluorescein angiography, or by direct retinal observation. In some embodiments, the reduction of retinal fluid (e.g., SRF and/or IRF) in the eye is determined by optical coherence tomography (OCT). In some embodiments, the reduction of retinal fluid (e.g., SRF and/or IRF) in the eye is determined by spectral domain OCT (SD-OCT). In some embodiments, the reduction of retinal fluid (e.g., SRF and/or IRF) in the eye is determined by OCT angiography. In some embodiments, the reduction of retinal fluid (e.g., SRF and/or IRF) in the eye is determined by fluorescein angiography. In some embodiments, the reduction of retinal fluid (e.g., SRF and/or IRF) in the eye is determined by direct retinal observation.

In some embodiments, treatment of an ocular neovascular disease in an individual after administration of the unit dose of rAAV particles to the one eye and/or the contralateral eye is assessed based on the level of retinal fluid (e.g., intraretinal fluid (IRF) and/or subretinal fluid (SRF)) compared the level of retinal fluid (e.g., SRF and/or IRF) prior to administration of the unit dose of rAAV particles to the one eye and/or the contralateral eye (e.g., as described above). In some embodiments, the retinal fluid is subretinal fluid (SRF) or intraretinal fluid (IRF). In some embodiments, the retinal fluid is subretinal fluid (SRF). In some embodiments, the retinal fluid is intraretinal fluid (IRF). In some embodiments, treatment of an ocular neovascular disease in an individual after administration of the unit dose of rAAV particles to the one eye and/or the contralateral eye is determined if a reduction in retinal fluid (e.g., IRF and/or SRF) is observed after administration of the unit dose of rAAV particles to the one eye and/or the contralateral eye compared to the levels of retinal fluid (e.g., IRF and/or SRF) prior to administration of the unit dose of rAAV particles in the one eye and/or the contralateral eye (e.g., as described above). In some embodiments, the ocular neovascular disease is wAMD.

In some embodiments, the administering the unit dose of rAAV particles to the one eye and/or to the contralateral eye of the individual results in maintenance or a decrease of retinal thickness compared to the retinal thickness prior to administration of the unit dose of rAAV particles. In some embodiments, the administering the unit dose of rAAV particles to the one eye and/or to the contralateral eye of the individual results in a decrease of retinal thickness compared to the retinal thickness prior to administration of the unit dose of rAAV particles. In some embodiments, retinal thickness is central subfield thickness (CST) or central retinal thickness (CRT). In some embodiments, the administering the unit dose of rAAV particles to the one eye and/or to the contralateral eye of the individual results in a decrease of retinal thickness of more than any of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% compared to the retinal thickness prior to administration of the unit dose of rAAV particles. In some embodiments, the retinal thickness (e.g., CST or CRT) is determined by OCT or SD-OCT.

In some embodiments, the administering the unit dose of rAAV particles to the one eye and/or to the contralateral eye of the individual results in a decrease of central retinal thickness (CRT) or central subfield thickness (CST) of about 10 μm to about 100 μm (e.g., more than any of about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or more). In some embodiments, the administering the unit dose of rAAV particles to the one eye and/or to the contralateral eye of the individual results in a decrease of central retinal thickness (CRT) or central subfield thickness (CST) of about 5 μm to about 50 μm (e.g., any of about 5 μm, about 10 μm, about 15 μm, about 20 μm, about 25 μm, about 30 μm, about 35 μm, about 40 μm, about 45 μm, or about 50 μm). In some embodiments, the administering the unit dose of rAAV particles to the one eye and/or to the contralateral eye of the individual results in a decrease of central retinal thickness (CRT) or central subfield thickness (CST) of about 5 μm to about 40 μm (e.g., any of about 5 μm, about 10 μm, about 15 μm, about 20 μm, about 25 μm, about 30 μm, about 35 μm, or about 40 μm). In some embodiments, the administering the unit dose of rAAV particles to the one eye and/or to the contralateral eye of the individual results in a decrease of central retinal thickness (CRT) or central subfield thickness (CST) of about 5 μm to about 30 μm (e.g., any of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, or about 30 μm). In some embodiments, the administering the unit dose of rAAV particles to the one eye and/or to the contralateral eye of the individual results in a decrease of central retinal thickness (CRT) or central subfield thickness (CST) of about 5 μm to about 25 μm (e.g., any of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, or about 25 μm). In some embodiments, the administering the unit dose of rAAV particles to the one eye and/or to the contralateral eye of the individual results in a decrease of central retinal thickness (CRT) or central subfield thickness (CST) of about 5 μm to about 20 μm (e.g., any of about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, or about 20 μm). In some embodiments, the administering the unit dose of rAAV particles to the one eye and/or to the contralateral eye of the individual results in a decrease of central retinal thickness (CRT) or central subfield thickness (CST) of between about 18 μm to about 75 μm. In some embodiments, the administering the unit dose of rAAV particles to the one eye and/or to the contralateral eye of the individual results in a decrease of central retinal thickness (CRT) or central subfield thickness (CST) of about 18.5 μm. In some embodiments, the administering the unit dose of rAAV particles to the one eye and/or to the contralateral eye of the individual results in a decrease of central retinal thickness (CRT) or central subfield thickness (CST) of about 21.0 μm. In some embodiments, the administering the unit dose of rAAV particles to the one eye and/or to the contralateral eye of the individual results in a decrease of central retinal thickness (CRT) or central subfield thickness (CST) of about 8.3 μm. In some embodiments, the administering the unit dose of rAAV particles to the one eye and/or to the contralateral eye of the individual results in a decrease of central retinal thickness (CRT) or central subfield thickness (CST) of about 25.5 μm. In some embodiments, the administering the unit dose of rAAV particles to the one eye and/or to the contralateral eye of the individual results in a decrease of central retinal thickness (CRT) or central subfield thickness (CST) of about 24.8 μm. In some embodiments, the administering the unit dose of rAAV particles to the one eye and/or to the contralateral eye of the individual results in a decrease of central retinal thickness (CRT) or central subfield thickness (CST) of about 75 μm. In some embodiments, the administering the unit dose of rAAV particles to the one eye and/or to the contralateral eye of the individual results in a decrease of central retinal thickness (CRT) or central subfield thickness (CST) of about 100 μm or more (e.g., any of about 100 μm or more, about 110 μm or more, about 120 μm or more, about 130 μm or more, about 140 μm or more, about 150 μm or more, about 160 μm or more, about 170 μm or more, about 180 μm or more, about 190 μm or more, or about 200 μm or more). In some embodiments, the administering the unit dose of rAAV particles to the one eye and/or to the contralateral eye of the individual results in a decrease of central retinal thickness (CRT) or central subfield thickness (CST) of between about 10 μm to about 200 μm (e.g., any of about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 50 μm to about 200 μm (e.g., any of about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 60 μm to about 200 μm (e.g., any of about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 70 μm to about 200 μm (e.g., any of about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 80 μm to about 200 μm (e.g., any of about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 90 μm to about 200 μm (e.g., any of about 90 μm, about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 100 μm to about 200 μm (e.g., any of about 100 μm, about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 110 μm to about 200 μm (e.g., any of about 110 μm, about 120 μm, about 130 μm, about 140 μm, about 150 μm, about 160 μm, about 170 μm, about 180 μm, about 190 μm, or about 200 μm). In some embodiments, the administering the unit dose of rAAV particles to the one eye and/or to the contralateral eye of the individual results in a decrease of central retinal thickness (CRT) or central subfield thickness (CST) of between about 115 μm to about 200 μm (e.g., any of about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 120 μm to about 200 μm (e.g., any of about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 125 μm to about 200 μm (e.g., any of about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 130 μm to about 200 μm (e.g., any of about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 135 μm to about 200 μm (e.g., any of about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 140 μm to about 200 μm (e.g., any of about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 145 μm to about 200 μm (e.g., any of about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 150 μm to about 200 μm (e.g., any of about 150 μm, about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 155 μm to about 200 μm (e.g., any of about 155 μm, about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 160 μm to about 200 μm (e.g., any of about 160 μm, about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 165 μm to about 200 μm (e.g., any of about 165 μm, about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 170 μm to about 200 μm (e.g., any of about 170 μm, about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 175 μm to about 200 μm (e.g., any of about 175 μm, about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of between about 180 μm to about 200 μm (e.g., any of about 180 μm, about 185 μm, about 190 μm, about 195 μm, or about 200 μm). In some embodiments, the administering the unit dose of rAAV particles to the one eye and/or to the contralateral eye of the individual results in a decrease of central retinal thickness (CRT) or central subfield thickness (CST) of about 21.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 8.3 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 26.2 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 24.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 40.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 30.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 118.6 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 119.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 137.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 152.7 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 153.3 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 149.8 μm compared to the central retinal thickness (CRT) or central subfield thickness (CST) prior to administration of the unit dose of rAAV particles.

In some embodiments, the administering the unit dose of rAAV particles to the one eye and/or to the contralateral eye of the individual results in a change in the central retinal thickness (CRT) or central subfield thickness (CST) of between about-200 μm to about +40 μm (e.g., any about −200 μm, about −180 μm, about −160 μm, about −140 μm, about −120 μm, about −100 μm, about −80 μm, about −60 μm, about −40 μm, about −20 μm, about 0 μm, about +5 μm, about +10 μm, about +15 μm, about +20 μm, about +25 μm, about +30 μm, about +35 μm, or about +40 μm) compared to the retinal thickness prior to administration of the unit dose of rAAV particles. In some embodiments, the administering the unit dose of rAAV particles to the one eye and/or to the contralateral eye of the individual results in a decrease in the central retinal thickness (CRT) or central subfield thickness (CST) of any of about 8 μm, about 11 μm, about 16 μm, about 29 μm, about 33 μm, about 38 μm, about 55 μm, about 61 μm, or about 117 μm. In some embodiments, the administering the unit dose of rAAV particles to the one eye and/or to the contralateral eye of the individual results in a decrease in the central retinal thickness (CRT) or central subfield thickness (CST) of any of about 27.8 μm or about 30.8 μm. In some embodiments, the administering the unit dose of rAAV particles to the one eye and/or to the contralateral eye of the individual results in an increase in the central retinal thickness (CRT) or central subfield thickness (CST) of any of about 4 μm, about 12 μm, or about 32 μm. In some embodiments, the administering the unit dose of rAAV particles to the one eye and/or to the contralateral eye of the individual results in a change in the central retinal thickness (CRT) or central subfield thickness (CST) of about −21.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −8.3 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −26.2 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −24.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −40.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −30.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −118.6 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −119.0 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −137.8 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −152.7 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −153.3 μm compared to the central retinal thickness (CRT) or central subfield thickness (CST) prior to administration of the unit dose of rAAV particles. In some embodiments, the administering the unit dose of rAAV particles to the one eye and/or to the contralateral eye of the individual results in a change in the central retinal thickness (CRT) or central subfield thickness (CST) of about −149.8 μm compared to the central retinal thickness (CRT) or central subfield thickness (CST) prior to administration of the unit dose of rAAV particles.

In some embodiments, the change (e.g., the decrease) in the central retinal thickness (CRT) or central subfield thickness (CST) compared to the central retinal thickness (CRT) or central subfield thickness (CST) prior to administration of the unit dose of rAAV particles is present at any of about 1 day, about 1 week, about 2 weeks, about 4 weeks, about 8 weeks, about 16 weeks, about 24 weeks, about 30 weeks, about 32 weeks, about 34 weeks, about 40 weeks, about 44 weeks, about 48 weeks, about 52 weeks, or more after administration of the unit dose of rAAV particles to the one eye and/or to the contralateral eye of the individual. In some embodiments, the change (e.g., the decrease) in the central retinal thickness (CRT) or central subfield thickness (CST) compared to the central retinal thickness (CRT) or central subfield thickness (CST) prior to administration of the unit dose of rAAV particles is present at any of about 1 day, about 1 week, about 2 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, about 104 weeks, about 108 weeks, or more after administration of the unit dose of rAAV particles to the one eye and/or to the contralateral eye of the individual.

In some embodiments, the administering the unit dose of rAAV particles to the one eye and/or to the contralateral eye of the individual results in a decrease of central retinal thickness (CRT) or central subfield thickness (CST) of more than any of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% compared to the retinal thickness prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of at least about 10% compared to the retinal thickness prior to administration of the unit dose of rAAV particles. In some embodiments, the administering the unit dose of rAAV particles to the one eye and/or to the contralateral eye of the individual results in a decrease of central retinal thickness (CRT) or central subfield thickness (CST) of about 15% or more compared to the retinal thickness prior to administration of the unit dose of rAAV particles.

In some embodiments, the administering the unit dose of rAAV particles to the one eye and/or to the contralateral eye of the individual results in an increase of central retinal thickness (CRT) or central subfield thickness (CST) of less than about 40 μm (e.g., any of less than about 40 μm, less than about 35 μm, less than about 30 μm, less than about 25 μm, less than about 20 μm, less than about 15 μm, less than about 10 μm, less than about 5 μm, less than about 1 μm, or less) compared to the retinal thickness prior to administration of the unit dose of rAAV particles. In some embodiments, the administering the unit dose of rAAV particles to the one eye and/or to the contralateral eye of the individual results in an increase of central retinal thickness (CRT) or central subfield thickness (CST) of about 32 μm or less compared to the retinal thickness prior to administration of the unit dose of rAAV particles.

In some embodiments, the maintenance, the decrease, or the increase of retinal thickness compared to the retinal thickness prior to administration of the unit dose of rAAV particles is present at about 30 weeks or more after administration of the unit dose of rAAV particles to the one eye and/or to the contralateral eye of the individual. In some embodiments, the maintenance, the decrease, or the increase of retinal thickness compared to the retinal thickness prior to administration of the unit dose of rAAV particles is present at any of about 30 weeks, about 34 weeks, about 44 weeks, about 6 months, about 1 year, about 1.5 years, about 2 years, about 3 years, about 5 years, about 10 years, or more, after administration of the unit dose of rAAV particles to the one eye and/or to the contralateral eye of the individual.

In some embodiments, the administering the unit dose of rAAV particles to the one eye and/or to the contralateral eye of the individual results in maintenance or a decrease in macular volume compared to the macular volume prior to administration of the unit dose of rAAV particles. In some embodiments, the administering the unit dose of rAAV particles to the one eye and/or to the contralateral eye of the individual results in a decrease in macular volume compared to the macular volume prior to administration of the unit dose of rAAV particles. In some embodiments, the administering the unit dose of rAAV particles to the one eye and/or to the contralateral eye of the individual results in a decrease in macular volume of more than any of about 5%, about 10%, about 15%, about 20%, about 25%, about 30%, about 35%, about 40%, about 45%, or about 50% compared to the macular volume prior to administration of the unit dose of rAAV particles. In some embodiments, the administering the unit dose of rAAV particles to the one eye and/or to the contralateral eye of the individual results in a decrease in macular volume of at least about 10% compared to the macular volume prior to administration of the unit dose of rAAV particles. In some embodiments, the macular volume is determined by OCT or SD-OCT. In some embodiments, the administering the unit dose of rAAV particles to the one eye and/or to the contralateral eye of the individual results in a decrease in macular volume of at least about 10% compared to the macular volume prior to administration of the unit dose of rAAV particles. In some embodiments, the administering the unit dose of rAAV particles to the one eye and/or to the contralateral eye of the individual results in a decrease in macular volume of about 15% or more compared to the macular volume prior to administration of the unit dose of rAAV particles. In some embodiments, the macular volume is determined by OCT or SD-OCT.

In some embodiments, the maintenance or the decrease in macular volume compared to the macular volume prior to administration of the unit dose of rAAV particles is present at about 30 weeks or more after administration of the unit dose of rAAV particles to the one eye and/or to the contralateral eye of the individual. In some embodiments, the maintenance or the decrease in macular volume compared to the macular volume prior to administration of the unit dose of rAAV particles is present at any of about 30 weeks, about 34 weeks, about 44 weeks, about 6 months, about 1 year, about 1.5 years, about 2 years, about 3 years, about 5 years, about 10 years, or more after administration of the unit dose of rAAV particles to the one eye and/or to the contralateral eye of the individual.

In some embodiments, the administering the unit dose of rAAV particles to the one eye and/or to the contralateral eye of the individual results in maintenance or an improvement of visual acuity compared to the visual acuity prior to administration of the unit dose of rAAV particles. In some embodiments, the administering the unit dose of rAAV particles to the one eye and/or to the contralateral eye of the individual results in an improvement of visual acuity compared to the visual acuity prior to administration of the unit dose of rAAV particles. In some embodiments, the administering the unit dose of rAAV particles to the one eye and/or to the contralateral eye of the individual results in an improvement of visual acuity of more than any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, compared to the visual acuity prior to administration of the unit dose of rAAV particles. In some embodiments, visual acuity is best corrected visual acuity (BCVA). In some embodiments, the administering the unit dose of rAAV particles to the one eye and/or to the contralateral eye of the individual results in an improvement of BCVA compared to the BCVA prior to administration of the unit dose of rAAV particles. In some embodiments, BCVA is expressed as an ETDRS score, which corresponds to the number of letters correctly read (Vitale et al., (2016) JAMA Opthalmol 134 (9): 1041:1047).

In some embodiments, the administering the unit dose of rAAV particles to the one eye and/or to the contralateral eye of the individual results in an improvement of BCVA of at least 15 ETDRS letters (Vitale et al., (2016) JAMA Opthalmol 134 (9): 1041:1047) (e.g., at least about 15, at least about 20, at least about 30, at least about 40, at least about 50, at least about 60, or about 70 letters) compared to the BCVA prior to administration of the unit dose of rAAV particles. In some embodiments, the administering the unit dose of rAAV particles to the one eye and/or to the contralateral eye of the individual results in an improvement of BCVA of between about 1 to about 15 (e.g., any of about 1, about 2, about 3, about 4, about 5, about 6, about 7, about 8, about 9, about 10, about 11, about 12, about 13, about 14, or about 15) ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 5 ETDRS letters compared to the BCVA prior to administration of the unit dose of rAAV particles. In some embodiments, the administering the unit dose of rAAV particles to the one eye and/or to the contralateral eye of the individual results in an improvement of BCVA of any of about 1, about 2, about 3, about 4, about 5, about 6, or about 7 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 3 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 4 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 5.1 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 6.4 ETDRS letters or more compared to the BCVA prior to administration of the unit dose of rAAV particles. In some embodiments, the administering the unit dose of rAAV particles to the one eye and/or to the contralateral eye of the individual results in an improvement of BCVA of about 6.8 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 8.8 ETDRS letters compared to the BCVA prior to administration of the unit dose of rAAV particles. In some embodiments, the administering the unit dose of rAAV particles to the one eye and/or to the contralateral eye of the individual results in an improvement of BCVA of about 2.3 ETDRS letters compared to the BCVA prior to administration of the unit dose of rAAV particles.

In some embodiments, the administering the unit dose of rAAV particles to the one eye and/or to the contralateral eye of the individual results in maintenance of BCVA, wherein the individual loses fewer than 15 ETDRS letters (Vitale et al., (2016) JAMA Opthalmol 134 (9): 1041:1047) (e.g., any of 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses any of about 1, about 2, about 3, about 4, about 5, about 6, about 7, about 8, or about 9 ETDRS letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses 0 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 1 letter compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2.7 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2.8 letters compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 2 letters or less compared to the BCVA prior to administration of the unit dose of rAAV particles. In some embodiments, the administering the unit dose of rAAV particles to the one eye and/or to the contralateral eye of the individual results in maintenance of BCVA, wherein the individual loses about 3.2 letters or less compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 15 to about 0 letters (e.g., any of about 15, about 14, about 13, about 12, about 11, about 10, about 9, about 8, about 7, about 6, about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 10 to about 0 letters (e.g., any of about 10, about 9, about 8, about 7, about 6, about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 5 to about 0 letters (e.g., any of about 5, about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 4 to about 0 letters (e.g., any of about 4, about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 3 to about 0 letters (e.g., any of about 3, about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 2 to about 0 letters (e.g., any of about 2, about 1, or 0 letters) compared to the BCVA prior to administration of the unit dose of rAAV particles. In some embodiments, the administering the unit dose of rAAV particles to the one eye and/or to the contralateral eye of the individual results in maintenance of BCVA, wherein the individual loses between about 1 to about 0 letters compared to the BCVA prior to administration of the unit dose of rAAV particles.

In some embodiments, the administering the unit dose of rAAV particles to the one eye and/or to the contralateral eye of the individual results in a change in BCVA of between about −20 to +7 or more (e.g., any of −20, −19, −18, −17, −16, −15, −14, −13, −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in an increase in BCVA of about any of 16, 7 or 5 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about any of 19, 14, 7, 6, 5, 4, 3, 2, or 1 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about 4.8 or about 0.8 ETDRS letters compared to the BCVA prior to administration of the unit dose of rAAV particles. In some embodiments, the administering the unit dose of rAAV particles to the one eye and/or to the contralateral eye of the individual results in a decrease in BCVA of about 2 ETDRS letters or less compared to the BCVA prior to administration of the unit dose of rAAV particles. In some embodiments, the administering the unit dose of rAAV particles to the one eye and/or to the contralateral eye of the individual results in a decrease in BCVA of about 3.2 ETDRS letters or less compared to the BCVA prior to administration of the unit dose of rAAV particles.

In some embodiments, the administering the unit dose of rAAV particles to the one eye and/or to the contralateral eye of the individual results in a change in BCVA of between about −15 to +7 or more (e.g., any of −15, −14, −13, −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −10 to +7 or more (e.g., any of −10, −9, −8, −7, −6, −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −5 to +7 or more (e.g., any of −5, −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −4 to +7 or more (e.g., any of −4, −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −3 to +7 or more (e.g., any of −3, −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −2 to +7 or more (e.g., any of −2, −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about −1 to +7 or more (e.g., any of −1, 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about 0 to +7 or more (e.g., any of 0, +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +1 to +7 or more (e.g., any of +1, +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +2 to +7 or more (e.g., any of +2, +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +3 to +7 or more (e.g., any of +3, +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +4 to +7 or more (e.g., any of +4, +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of between about +5 to +7 or more (e.g., any of +5, +6, +7, +8, +9, +10, +11, +12, +13, +14, +15, +16, +17, +18, +19, +20, or more) ETDRS letters, compared to the BCVA prior to administration of the unit dose of rAAV particles. In some embodiments, the administering the unit dose of rAAV particles to the one eye and/or to the contralateral eye of the individual results in a change in BCVA of about +6 or about +7 ETDRS letters, compared to the BCVA prior to administration of the unit dose of rAAV particles.

In some embodiments, administration of the unit dose of rAAV particles to the one eye and/or to the contralateral eye of the individual results in transient inflammation (e.g., inflammation driven by aqueous cells and/or vitreous cells, aqueous flare, posterior synchiae, poor pupil dilation). In some embodiments, administration of the unit dose of rAAV particles to the one eye and/or to the contralateral eye of the individual results in inflammation (e.g., inflammation driven by aqueous cells and/or vitreous cells, aqueous flare, posterior synchiae, poor pupil dilation) that is improved after administration of oral and/or topical steroid treatment and/or mydryatics. In some embodiments, administration of the unit dose of rAAV particles to the one eye and/or to the contralateral eye of the individual results in inflammation (e.g., inflammation driven by aqueous cells and/or vitreous cells) that resolves after administration of oral and/or topical steroid treatment. Inflammation (e.g., inflammation driven by aqueous cells and/or vitreous cells, aqueous flare, posterior synchiae, poor pupil dilation) may be measured using any method known in the art, such as the slit lamp exam.

In some embodiments, the maintenance or the improvement of visual acuity (e.g., BCVA) compared to the visual acuity prior to administration of the unit dose of rAAV particles is present at any of about 1 day, about 1 week, about 2 weeks, about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, about 76 weeks, about 80 weeks, about 84 weeks, about 88 weeks, about 92 weeks, about 96 weeks, about 100 weeks, about 104 weeks, about 108 weeks, or more, after administration of the unit dose of rAAV particles to the one eye and/or to the contralateral eye of the individual. In some embodiments, the maintenance or the improvement of visual acuity (e.g., BCVA) compared to the visual acuity prior to administration of the unit dose of rAAV particles is present at about 30 weeks or more after administration of the unit dose of rAAV particles to the one eye and/or to the contralateral eye of the individual. In some embodiments, the maintenance or the improvement of visual acuity (e.g., BCVA) compared to the visual acuity prior to administration of the unit dose of rAAV particles is present at any of about 30 weeks, about 34 weeks, about 44 weeks, about 6 months, about 1 year, about 1.5 years, about 2 years, about 3 years, about 5 years, about 10 years, or more, after administration of the unit dose of rAAV particles to the one eye and/or to the contralateral eye of the individual.

In some embodiments, treatment of an ocular neovascular disease in an individual after administration of the unit dose of rAAV particles in the one eye and/or the contralateral eye is assessed based on best corrected visual acuity (BCVA) in the one eye and/or the contralateral eye. In some embodiments, BCVA is expressed as an ETDRS score, which corresponds to the number of letters correctly read (Vitale et al., (2016) JAMA Opthalmol 134 (9): 1041:1047). In some embodiments, an individual is determined to have maintenance of vision and/or visual acuity if the individual loses fewer than 15 letters in an ETDRS score (e.g., any of 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to prior to administration of the unit dose of rAAV particles in the one eye and/or the contralateral eye. In some embodiments, an individual is determined to have an improvement of vision and/or visual acuity if the individual gains at least 15 letters (e.g., any of at least about 15, at least about 20, at least about 30, at least about 40, at least about 50, at least about 60, or about 70 letters) comparted to prior to administration of the unit dose of rAAV particles in the one eye and/or the contralateral eye.

In some embodiments, treatment of an ocular neovascular disease in an individual after administration of the unit dose of rAAV particles in the one eye and/or the contralateral eye is assessed based on central subfield thickness (CST) or central retinal thickness (CRT) in the one eye and/or the contralateral eye. In some embodiments, CST or CRT is determined by SD-OCT. In some embodiments, treatment of an ocular neovascular disease in an individual after administration of the unit dose of rAAV particles in the one eye and/or the contralateral eye is determined if the CST or CRT assessed by SD-OCT is decreased after administration of the unit dose of rAAV particles in the one eye and/or the contralateral eye compared to prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if the CST or CRT assessed by SD-OCT is maintained after administration of the unit dose of rAAV particles in the one eye and/or the contralateral eye compared to prior to administration of the unit dose of rAAV particles in the one eye and/or the contralateral eye.

In some embodiments, treatment of an ocular neovascular disease in an individual after administration of the unit dose of rAAV particles in the one eye and/or the contralateral eye is assessed based on macular volume in the one eye and/or the contralateral eye. In some embodiments, macular volume is determined by SD-OCT. In some embodiments, treatment of an ocular neovascular disease in an individual after administration of the unit dose of rAAV particles in the one eye and/or the contralateral eye is determined if the macular volume assessed by SD-OCT is decreased after administration of the unit dose of rAAV particles in the one eye and/or the contralateral eye compared to prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if the macular volume assessed by SD-OCT is maintained after administration of the unit dose of rAAV particles in the one eye and/or the contralateral eye compared to prior to administration of the unit dose of rAAV particles in the one eye and/or the contralateral eye.

In some embodiments, treatment of an ocular neovascular disease in an individual after administration of the unit dose of rAAV particles in the one eye and/or the contralateral eye is assessed based on retinal thickness (e.g., central retinal thickness (CRT) or central subfield thickness (CST)) and macular volume in the one eye and/or the contralateral eye. In some embodiments, CST and macular volume are determined by SD-OCT. In some embodiments, treatment of an ocular neovascular disease in an individual after administration of the unit dose of rAAV particles in the one eye and/or the contralateral eye is determined if the CST and macular volume assessed by SD-OCT are decreased after administration of the unit dose of rAAV particles in the one eye and/or the contralateral eye compared to prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if the CST and macular volume assessed by SD-OCT are maintained after administration of the unit dose of rAAV particles in the one eye and/or the contralateral eye compared to prior to administration of the unit dose of rAAV particles in the one eye and/or the contralateral eye.

In some embodiments, treatment of an ocular neovascular disease in an individual after administration of the unit dose of rAAV particles in the one eye and/or the contralateral eye is assessed based on the number of rescue therapy treatments (e.g., aflibercept injections) required by the individual after administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if an individual requires less than one rescue therapy treatment (e.g., aflibercept injection) any of every 4 weeks, every 5 weeks, every 6 weeks, every 7 weeks, every 8 weeks, every 9 weeks, every 10 weeks, or more, after administration of the unit dose of rAAV particles in the one eye and/or the contralateral eye.

In some embodiments, treatment of an ocular neovascular disease in an individual after administration of the unit dose of rAAV particles in the one eye and/or the contralateral eye is determined if an individual does not require any rescue therapy treatment (e.g., aflibercept injection) for any of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 15 weeks, at least 20 weeks, at least 30 weeks, at least 40 weeks, at least 50 weeks, at least 60 weeks, at least 70 weeks, at least 80 weeks, at least 90 weeks, at least 100 weeks, at least 110 weeks, or more, after administration of the unit dose of rAAV particles in the one eye and/or the contralateral eye.

In some embodiments, the individual does not require any rescue therapy treatment (e.g., aflibercept injection) for any of at least about 24 months, at least about 23 months, at least about 22 months, at least about 21 months, at least about 20 months, at least about 19 months, at least about 18 months, at least about 17 months, at least about 16 months, at least about 15 months, at least about 14 months, at least about 13 months, at least about 12 months, at least about 11 months, at least about 10 months, at least about 9 months, at least about 8 months, at least about 7 months, at least about 6 months, at least about 5 months, at least about 4 months, at least about 3 months, at least about 2 months, at least about 1 month, at least about 3 weeks, at least about 2 weeks, or at least about 1 week after administration of the unit dose of rAAV particles in the one eye and/or the contralateral eye. In some embodiments, the individual does not require any rescue therapy treatment (e.g., aflibercept injection) for at least about 12 months after administration of the unit dose of rAAV particles in the one eye and/or the contralateral eye. In some embodiments, the individual does not require any rescue therapy treatment (e.g., aflibercept injection) for at least about 10 months after administration of the unit dose of rAAV particles in the one eye and/or the contralateral eye. In some embodiments, the individual does not require any rescue therapy treatment (e.g., aflibercept injection) for at least about 7 months after administration of the unit dose of rAAV particles in the one eye and/or the contralateral eye. In some embodiments, the individual does not require any rescue therapy treatment (e.g., aflibercept injection) for at least about 6 months after administration of the unit dose of rAAV particles in the one eye and/or the contralateral eye. In some embodiments, the individual does not require any rescue therapy treatment (e.g., aflibercept injection) for at least about 2 months after administration of the unit dose of rAAV particles in the one eye and/or the contralateral eye. In some embodiments, the individual does not require any rescue therapy treatment (e.g., aflibercept injection) for at least about 1 month after administration of the unit dose of rAAV particles in the one eye and/or the contralateral eye.

In some embodiments, administration of a single unit dose of rAAV particles to the one eye and/or to the contralateral eye of a plurality of individuals results in at least about 50% (e.g., any of at least about 50%, at least about 55%, at least about 60%, at least about 65%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 67% (e.g., any of at least about 67%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 50% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 80% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in at least about 82% of the individuals in the plurality not requiring an anti-VEGF rescue treatment (e.g., aflibercept injection). In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection).

In some embodiments, administration of a single unit dose of rAAV particles to the one eye and/or to the contralateral eye of a plurality of individuals results in at least about 50% (e.g., any of at least about 50%, at least about 55%, at least about 60%, at least about 65%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more after administration of the rAAV particles. In some embodiments, administration of a single unit dose of rAAV particles to the one eye and/or to the contralateral eye of a plurality of individuals results in at least about 50% of the individuals in the plurality not requiring an anti-VEGF rescue treatment (e.g., aflibercept injection) for about 52 weeks or more, or about 56 weeks or more, after administration of the rAAV particles. In some embodiments, administration of a single unit dose of rAAV particles to the one eye and/or to the contralateral eye of a plurality of individuals results in at least about 67% (e.g., any of at least about 67%, at least about 70%, at least about 75%, at least about 80%, at least about 85%, at least about 90%, at least about 95%, at least about 99%, or 100%) of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles. In some embodiments, administration of a single unit dose of rAAV particles to the one eye and/or to the contralateral eye of a plurality of individuals results in at least about 78% of the individuals in the plurality not requiring an anti-VEGF rescue treatment (e.g., aflibercept injection) for about 20 weeks or more, or about 36 weeks or more, after administration of the rAAV particles. In some embodiments, administration of a single unit dose of rAAV particles to the one eye and/or to the contralateral eye of a plurality of individuals results in at least about 80% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a single unit dose of rAAV particles to the one eye and/or to the contralateral eye of a plurality of individuals results in at least about 82% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for at least about 4 weeks after administration of the rAAV particles, e.g., any of any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more after administration of the rAAV particles. In some embodiments, administration of a single unit dose of rAAV particles to the one eye and/or to the contralateral eye of a plurality of individuals results in 100% of the individuals in the plurality not requiring an anti-VEGF rescue treatment (e.g., aflibercept injection) for any of about 64 weeks or more, 72 weeks or more, or 84 weeks or more, after administration of the rAAV particles.

In some embodiments, administration of a unit dose of rAAV particles in the one eye and/or the contralateral eye of a plurality of individuals results in about 78% or less (e.g., any of about 78% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in about 50% or less (e.g., any of about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 30% (e.g., less than any of about 30%, about 25%, about 20%, about 15%, about 10%, about 5%, about 2.5%, about 1%, or about 0.5%) of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 30% of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in less than about 20% of the individuals in the plurality requiring any rescue treatment (e.g., aflibercept injection) in the one eye and/or the contralateral eye. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye.

In some embodiments, administration of a unit dose of rAAV particles in the one eye and/or the contralateral eye of a plurality of individuals results in about 78% or less (e.g., any of about 78% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2.5% or less, about 1% or less, or about 0.5% or less) of the individuals in the plurality requiring any rescue treatment (e.g., aflibercept injection) for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles. In some embodiments, administration of a unit dose of rAAV particles in the one eye and/or the contralateral eye of a plurality of individuals results in less than about 30% (e.g., less than any of about 30%, about 25%, about 20%, about 15%, about 10%, about 5%, about 2.5%, about 1%, or about 0.5%) of the individuals in the plurality requiring any rescue treatment (e.g., aflibercept injection) for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a unit dose of rAAV particles in the one eye and/or the contralateral eye of a plurality of individuals results in less than about 30% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more, after administration of the rAAV particles. In some embodiments, administration of a unit dose of rAAV particles in the one eye and/or the contralateral eye of a plurality of individuals results in less than about 20% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye for at least about 20 weeks after administration of the rAAV particles, e.g., any of at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 66 weeks, or more after administration of the rAAV particles. In some embodiments, administration of a unit dose of rAAV particles in the one eye and/or the contralateral eye of a plurality of individuals results in 0% of the individuals in the plurality requiring any rescue treatment (e.g., aflibercept injection) in the one eye and/or the contralateral eye for at least about 4 weeks after administration of the rAAV particles, e.g., any of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, at least about 96 weeks, at least about 100 weeks, at least about 104 weeks, at least about 108 weeks, or more, after administration of the rAAV particles.

In some embodiments, administration of the unit dose of rAAV particles to the one eye and/or to the contralateral eye of a plurality of individuals results in a reduction in the mean annualized anti-VEGF injection rate of any of at least about 80%, at least about 85%, at least about 87%, at least about 90%, at least about 95%, at least about 99%, or 100%, compared to the mean annualized anti-VEGF injection rate prior to administration of the unit dose of rAAV particles. In some embodiments, administration of the unit dose of rAAV particles to the one eye and/or to the contralateral eye of a plurality of individuals results in a reduction in the mean annualized anti-VEGF injection rate of about 87% or more compared to the mean annualized anti-VEGF injection rate prior to administration of the unit dose of rAAV particles. In some embodiments, administration of the unit dose of rAAV particles to the one eye and/or to the contralateral eye of a plurality of individuals results in a reduction in the mean annualized anti-VEGF injection rate of 100% compared to the mean annualized anti-VEGF injection rate prior to administration of the unit dose of rAAV particles.

In some embodiments, the mean annualized anti-VEGF injection rate prior to administration of the unit dose of rAAV particles is calculated according to the formula:

Annualized rate prior to administration of the unit dose of rAAV particles=(number of anti-VEGF injections in 12 months prior to administration of the unit dose of rAAV particles)/(days from the first anti-VEGF injection in the past 12 months prior to administration of the unit dose of rAAV particles to the administration of the unit dose of rAAV particles/365.25)

In some embodiments, the mean annualized anti-VEGF injection rate after administration of the unit dose of rAAV particles is calculated according to the formula:

Annualized rate after administration of the unit dose of rAAV particles=(number of anti-VEGF injections since administration of the unit dose of rAAV particles)/(days from administration of the unit dose of rAAV particles/365.25).

In some embodiments, an individual is determined to require a rescue treatment (e.g., anti-VEGF intravitreal injection, such as aflibercept injection) after administration of the rAAV particles if the individual exhibits loss of 10 or more letters in BCVA (e.g., using the ETDRS protocol) in the one eye and/or the contralateral eye administered the rAAV particles that is attributed to intraretinal or subretinal fluid (e.g., as determined by SD-OCT) compared to the BCVA in the one eye and/or the contralateral eye administered the rAAV particles prior to administration of the rAAV particles. In some embodiments, an individual is determined to require a rescue treatment (e.g., anti-VEGF intravitreal injection, such as aflibercept injection) after administration of the rAAV particles if the individual exhibits an increase in central subfield thickness (e.g., CST or CRT) greater than 75 μm in the one eye and/or the contralateral eye administered the rAAV particles compared to the central subfield thickness in the one eye and/or the contralateral eye administered the rAAV particles prior to administration of the rAAV particles, e.g., as determined by SD-OCT. In some embodiments, an individual is determined to require a rescue treatment (e.g., anti-VEGF intravitreal injection, such as aflibercept injection) after administration of the rAAV particles if the individual exhibits vision-threatening hemorrhage due to AMD in the one eye and/or the contralateral eye administered the rAAV particles.

In some embodiments, a rescue treatment comprises administration of a standard of care anti-VEGF therapy. Such standard of care anti-VEGF therapy comprises one or more anti-VEGF treatments (e.g., anti-VEGF intravitreal injections). In some embodiments, a rescue treatment comprises one or more aflibercept IVT injections. In some embodiments, a rescue treatment comprises one or more aflibercept IVT injections comprising about 2 mg of aflibercept.

In some embodiments, treatment of an ocular neovascular disease in an individual after administration of the unit dose of rAAV particles in the one eye and/or the contralateral eye is assessed based on the level of retinal fluid compared the level of retinal fluid prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if a reduction in retinal fluid is observed after administration of the unit dose of rAAV particles in the one eye and/or the contralateral eye compared to the level of retinal fluid prior to administration of the unit dose of rAAV particles in the one eye and/or the contralateral eye. In some embodiments, the ocular neovascular disease is wAMD.

In some embodiments, treatment of an ocular neovascular disease in an individual after administration of the unit dose of rAAV particles in the one eye and/or the contralateral eye is assessed based on the resolution of pigment epithelial detachment (PED) compared to PED prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if resolution of PED after administration of the unit dose of rAAV particles in the one eye and/or the contralateral eye is observed, compared to PED prior to administration of the unit dose of rAAV particles in the one eye and/or the contralateral eye. In some embodiments, the ocular neovascular disease is wAMD.

In some embodiments, treatment of an ocular neovascular disease in an individual after administration of the unit dose of rAAV particles in the one eye and/or the contralateral eye is assessed based on choroidal neovascularization (CNV) lesion growth as determined by fluorescein angiography. In some embodiments, treatment of an ocular neovascular disease in an individual after administration of the unit dose of rAAV particles in the one eye and/or the contralateral eye is determined if CNV lesions shrink (e.g., by more than any of about 5%, about 10%, about 20%, about 30%, about 40%, about 50%, about 60%, about 70%, about 80%, or 100%) after administration of the unit dose of rAAV particles in the one eye and/or the contralateral eye compared to CNV lesions present prior to administration of the unit dose of rAAV particles in the one eye and/or the contralateral eye. In some embodiments, treatment of an ocular neovascular disease in an individual after administration of the unit dose of rAAV particles in the one eye and/or the contralateral eye is determined if CNV lesions do not grow (e.g., grow less than any of about 1%, about 2%, about 3%, about 4%, about 5%, about 6%, about 7%, about 8%, about 9%, about 10%, about 15%, or about 20%) after administration of the unit dose of rAAV particles in the one eye and/or the contralateral eye compared to CNV lesions present prior to administration of the unit dose of rAAV particles in the one eye and/or the contralateral eye. In some embodiments, the ocular neovascular disease is wAMD.

In some embodiments, treatment of an ocular neovascular disease in an individual after administration of the unit dose of rAAV particles in the one eye and/or the contralateral eye is assessed based on the anatomical features of the one eye and/or the contralateral eye based on any methods known in the art (e.g., SD-OCT, OCT, fluorescein angiography, digital color fundus photography, etc.). In some embodiments, treatment of an ocular neovascular disease in an individual after administration of the unit dose of rAAV particles in the one eye and/or the contralateral eye is determined if an improvement in anatomical features of the one eye and/or the contralateral eye is observed after administration of the unit dose of rAAV particles in the one eye and/or the contralateral eye. In some embodiments, the ocular neovascular disease is wAMD.

In some embodiments, treatment of an ocular neovascular disease in an individual after administration of the unit dose of rAAV particles in the one eye and/or the contralateral eye is assessed based on ophthalmologic examination, intraocular pressure (e.g., using a Goldmann applanation tonometer or Tono-pen), indirect ophthalmoscopy, examination of the one eye and/or the contralateral eye and adnexa, eyelid and/or pupil responsiveness, belpharoptosis, abnormal pupil shape, unequal pupils, abnormal reaction to light, afferent pupillary defects, slit-lamp examination (including of the eyelids, conjunctiva, cornea, lens, iris, and anterior chamber), posterior segment abnormalities of the vitreous, optic nerve, peripheral retina, and retinal vasculature, SD-OCT, fluorescein angiography, digital color fundus photography (including images of the retina, optic disc, and/or macula), aqueous humor sampling, vitreous humor sampling, OCT-angiography (OCT-A), refraction and/or visual acuity (BCVA). In some embodiments, SD-OCT is performed to evaluate retinal thickness (e.g., central retinal thickness or central subfield thickness), macular volume, and/or the presence of fluid (e.g., subretinal fluid or intraretinal fluid). In some embodiments, the ocular neovascular disease is wAMD.

The unit dose of rAAV particles may be administered to the one eye and/or to the contralateral eye of the individual by any method known in the art. For example, the unit dose of rAAV particles may be administered to the one eye and/or to the contralateral eye of the individual intraocularly, or by intravitreal injection. In some embodiments, the administration of the unit dose of rAAV particles to the one eye and/or to the contralateral eye of the individual is intraocular. In some embodiments, the administration of the unit dose of rAAV particles to the one eye and/or to the contralateral eye of the individual is by intravitreal injection (IVT) or subretinal injection. In some embodiments, the administration of the unit dose of rAAV particles to the one eye and/or to the contralateral eye of the individual is by IVT injection. In some embodiments, aseptic technique is employed to administer a unit dose of rAAV particles by intravitreal injection. In some embodiments, aseptic technique with providone-iodine is employed to administer a unit dose of rAAV particles by intravitreal injection.

In some embodiments, the individual has not received a prior treatment for an ocular neovascular disease. In some embodiments, the individual has not received a prior treatment in the one eye and/or the contralateral eye for an ocular neovascular disease. In some embodiments, the individual has not received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept). In some embodiments, the individual has not received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye. In some embodiments, the individual has not received a prior aflibercept treatment. In some embodiments, the individual has not received a prior aflibercept treatment in the one eye and/or the contralateral eye.

### Steroid Treatments

In some embodiments, the unit dose of rAAV particles is administered in combination with steroid treatment. In some embodiments, the steroid treatment is a corticosteroid treatment. Exemplary corticosteroids include, without limitation, aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone, and ulobetasol. In some embodiments, the steroid treatment is a systemic steroid treatment. In some embodiments, the steroid treatment is an oral steroid treatment. In some embodiments, the steroid treatment is an ophthalmic steroid treatment. In some embodiments, the ophthalmic steroid treatment is a topical steroid treatment (e.g. a drop), a periocular steroid treatment (e.g., subtenons, subconjunctival), an intravitreal steroid treatment, or a superchoroidal steroid treatment. In some embodiments, the topical steroid treatment is a difluprednate treatment, a medrysone treatment, a loteprednol treatment, a prednisolone treatment, a fluocinolone treatment, a triamcinolone treatment, a rimexolone treatment, a dexamethasone treatment, a fluorometholone treatment, a fluocinolone treatment, a rimexolone treatment, or a prednisone treatment. In some embodiments, the ophthalmic steroid treatment is a difluprednate treatment. In some embodiments, the steroid treatment is a prednisone treatment. In some embodiments, the steroid treatment is a difluprednate treatment.

In some embodiments, the steroid treatment comprises a systemic steroid treatment and a topical steroid treatment. In some embodiments, the systemic steroid treatment is an oral steroid treatment. In some embodiments, the systemic steroid treatment is a prednisone treatment. In some embodiments, the topical steroid treatment is a difluprednate treatment. In some embodiments, the systemic steroid treatment and the topical steroid treatment are administered simultaneously (e.g., on the same day). In some embodiments, the systemic steroid treatment and the topical steroid treatment are administered separately (e.g., on different days).

In some embodiments, the steroid treatment is an oral treatment such as, but not limited to, a prednisone treatment, methylprednisolone treatments, budesonide treatment, triamcinolone treatment, hydrocortisone treatment, and betamethasone treatment. In some embodiments, the oral steroid treatment (e.g., prednisone) is a daily steroid treatment for up to about 4 weeks, about 6 weeks, about 8 weeks, or about 9 weeks from administering the unit dose of rAAV particles. In some embodiments, the oral steroid treatment (e.g., prednisone) is administered at a dose of: 1ess than about 0.8 mg/kg/day to about 1.00 mg/kg/day for the week prior to through week two following administering the unit dose of rAAV particles, less than about 0.59 mg/kg/day to about 0.75 mg/kg/day on weeks three to five following administering the unit dose of rAAV particles, less than about 0.4 mg/kg/day to about 0.6 mg/kg/day on week six after administering the unit dose of rAAV particles, less than about 0.25 mg/kg/day to about 0.375 mg/kg/day on week seven after administering the unit dose of rAAV particles, less than about 0.14 mg/kg/day to about 0.28 mg/kg/day on week eight after administering the unit dose of rAAV particles, and less than about 0.063 mg/kg/day to about 0.125 mg/kg/day on week nine after administering the unit dose of rAAV particles. In some embodiments, the oral steroid treatment is administered at a dose of: 1ess than or equal to about 1.00 mg/kg/day for the week prior to through week two following administering the unit dose of rAAV particles, less than or equal to about 0.75 mg/kg/day on weeks three to five following administering the unit dose of rAAV particles, less than or equal to about 0.6 mg/kg/day on week six after administering the unit dose of rAAV particles, less than or equal to about 0.375 mg/kg/day on week seven after administering the unit dose of rAAV particles, less than or equal to about 0.28 mg/kg/day on week eight after administering the unit dose of rAAV particles, and less than or equal to about 0.125 mg/kg/day on week nine after administering the unit dose of rAAV particles. In some embodiments, the oral steroid treatment is administered at a dose of about 0.125 mg/kg/day to about 0.14 mg/kg/day on the first four days of week nine after administering the unit dose of rAAV particles and about 0.0625 mg/kg/day to about 0.070 mg/kg/day on the first four days of week nine after administering the unit dose of rAAV particles if the individual weighs 70 kg or more.

In some embodiments, the steroid is administered before, during, and/or after administration of the unit dose of rAAV particles. In some embodiments, the steroid is administered before, during, and after administration of the unit dose of rAAV particles. In some embodiments, the steroid is administered during, and after administration of the unit dose of rAAV particles. In some embodiments, the steroid is administered before administration of the unit dose of rAAV particles. In some embodiments, the steroid is administered during administration of the unit dose of rAAV particles. In some embodiments, the steroid is administered before and during administration of the unit dose of rAAV particles. In some embodiments, the steroid is administered after administration of the unit dose of rAAV particles. In some embodiments, the steroid is administered during and after administration of the unit dose of rAAV particles. In some embodiments, the steroid is administered before and/or after administration of the unit dose of rAAV particles. In some embodiments, the steroid is administered before and after administration of the unit dose of rAAV particles.

In some embodiments, the steroid treatment is a systemic steroid treatment. In some embodiments, the systemic steroid treatment is an oral steroid treatment.

In some embodiments, the steroid treatment is an oral prednisone treatment. In some embodiments, the oral prednisone treatment is initiated prior to administration of the unit dose of rAAV particles. In some embodiments, an initial oral prednisone treatment is administered at a dose of any of about 40 mg, about 45 mg, about 50 mg, about 55 mg, about 60 mg, about 65 mg, or about 70 mg of prednisone per day any of about 7 days, about 6 days, about 5 days, about 4 days, about 3 days, about 2 days, about 1 day, or 0 days before administration of the unit dose of rAAV particles, and is continued for any of about 3 days, about 4 days, about 5 days, about 6 days, about 7 days, about 8 days, about 9 days, or about 10 days, or more. In some embodiments, an initial oral prednisone treatment is administered at a dose of about 60 mg of prednisone per day about 3 days before administration of the unit dose of rAAV, and is continued for about 3 days.

In some embodiments, the initial oral prednisone treatment is followed by an oral prednisone treatment dose taper. In some embodiments, the oral prednisone treatment dose taper is administered at a dose of any of about 20 mg, about 25 mg, about 30 mg, about 35 mg, about 40 mg, about 45 mg, or about 50 mg of prednisone per day for a total of any of about 1 day, about 2 days, about 3 days, about 4 days, about 5 days, about 6 days, or about 7 days, followed by a dose of about 10 mg, about 15 mg, about 20 mg or about 25 mg of prednisone per day for any of about 1 day, about 2 days, about 3 days, or about 4 days, followed by a dose of about 5 mg, about 10 mg, or about 15 mg of prednisone per day for about 1 day, about 2 days, about 3 days, or about 4 days. In some embodiments, the prednisone dose taper is administered at a dose of any of about 40 mg of prednisone per day for 3 days, followed by a dose of about 20 mg of prednisone per day for 2 days, followed by a dose of about 10 mg of prednisone per day for 2 days.

In some embodiments, an initial oral prednisone treatment is initiated 3 days before administration of the unit dose of rAAV particles at a dose of 60 mg of prednisone per day for a total of 6 days, followed by a dose of 40 mg of prednisone per day for a total of 3 days, followed by a dose of 20 mg of prednisone per day for 2 days, followed by a dose of 10 mg of prednisone per day for 2 days.

In some embodiments, the steroid treatment is an oral treatment (e.g., prednisone). In some embodiments, the oral steroid treatment (e.g., prednisone) is a daily steroid treatment for up to about 4 weeks, about 6 weeks, about 8 weeks, or about 9 weeks from administering the unit dose of rAAV particles. In some embodiments, the oral steroid treatment (e.g., prednisone) is administered at a dose of: 1ess than about 0.8 mg/kg/day to about 1.00 mg/kg/day for the week prior to through week two following administering the unit dose of rAAV particles, less than about 0.59 mg/kg/day to about 0.75 mg/kg/day on weeks three to five following administering the unit dose of rAAV particles, less than about 0.4 mg/kg/day to about 0.6 mg/kg/day on week six after administering the unit dose of rAAV particles, less than about 0.25 mg/kg/day to about 0.375 mg/kg/day on week seven after administering the unit dose of rAAV particles, less than about 0.14 mg/kg/day to about 0.28 mg/kg/day on week eight after administering the unit dose of rAAV particles, and less than about 0.063 mg/kg/day to about 0.125 mg/kg/day on week nine after administering the unit dose of rAAV particles. In some embodiments, the oral steroid treatment is administered at a dose of: 1ess than or equal to about 1.00 mg/kg/day for the week prior to through week two following administering the unit dose of rAAV particles, less than or equal to about 0.75 mg/kg/day on weeks three to five following administering the unit dose of rAAV particles, less than or equal to about 0.6 mg/kg/day on week six after administering the unit dose of rAAV particles, less than or equal to about 0.375 mg/kg/day on week seven after administering the unit dose of rAAV particles, less than or equal to about 0.28 mg/kg/day on week eight after administering the unit dose of rAAV particles, and less than or equal to about 0.125 mg/kg/day on week nine after administering the unit dose of rAAV particles. In some embodiments, the oral steroid treatment is administered at a dose of about 0.125 mg/kg/day to about 0.14 mg/kg/day on the first four days of week nine after administering the unit dose of rAAV particles and about 0.0625 mg/kg/day to about 0.070 mg/kg/day on the first four days of week nine after administering the unit dose of rAAV particles if the individual weighs 70 kg or more.

In some embodiments, the steroid treatment is an ophthalmic steroid treatment. In some embodiments, the ophthalmic steroid treatment is a difluprednate treatment. In some embodiments, the steroid treatment is administered before, during, and/or after administration of the unit dose of rAAV particles. In some embodiments, the steroid treatment is administered before administration of the unit dose of rAAV particles. In some embodiments, the steroid treatment is administered during administration of the unit dose of rAAV particles. In some embodiments, the steroid treatment is administered after administration of the unit dose of rAAV particles. In some embodiments, the steroid treatment is administered before and during administration of the unit dose of rAAV particles. In some embodiments, the steroid treatment is administered before and after administration of the unit dose of rAAV particles. In some embodiments, the steroid treatment is administered during, and after administration of the unit dose of rAAV particles. In some embodiments, the steroid treatment is administered before, during, and after administration of the unit dose of rAAV particles.

In some embodiments, the steroid treatment is an ophthalmic steroid treatment, e.g., a topical steroid treatment. In some embodiments, the ophthalmic steroid treatment, e.g., a topical steroid treatment, is a daily steroid treatment for up to 4 weeks, up to 6 weeks, up to 8 weeks, up to 3 months, up to 4 months, up to 5 months, or up to 6 months after administration of the unit dose of rAAV particles. In some embodiments, the topical steroid treatment comprises about four administrations of topical steroid on about week 1, about three administrations of topical steroid on about week 2, about two administrations of topical steroid on about week 3, and about one administration of topical steroid on about week 4; timing starting with and following administration of the unit dose of rAAV particles. In some embodiments, the topical steroid treatment comprises about four administrations of topical steroid per day (i.e., QID) for about 3 weeks after administration of the unit dose of rAAV particles, followed by about 3 administrations of topical steroid per day (i.e., TID) for about 1 week, followed by about 2 administrations of topical steroid per day (i.e., BID) for about 1 week, and followed by about 1 administration of topical steroid per day (i.e., QD) for about 1 week. In some embodiments, the ophthalmic steroid treatment is extended at the discretion of the treating physician.

In some embodiments, the ophthalmic steroid is about 0.005% to about 0.5% difluprednate. In some embodiments, the ophthalmic steroid is any of about 0.005%, about 0.006%, about 0.007%, about 0.008%, about 0.009%, about 0.01%, about 0.02%, about 0.03%, about 0.4%, about 0.05%, about 0.06%, about 0.07%, about 0.08%, about 0.09%, or about 0.1% difluprednate. In some embodiments, the ophthalmic steroid is difluprednate 0.05%. In some embodiments, a dose of difluprednate 0.05% is one drop of ophthalmic solution. In some embodiments, one drop is about 50 μl (e.g., about 25 μl to about 50 μl, or about 50 μl to about 100 μl). In some embodiments, a dose of difluprednate comprises about 1 μg to about 5 μg, or about 2 μg to about 3 μg, or about 2.5 μg difluprednate. In some embodiments, a dose of difluprednate comprises about 2.5 μg difluprednate.

In some embodiments, the topical steroid treatment comprises a 7-week topical steroid treatment, e.g., 0.05% difluprednate. In some embodiments, the topical steroid treatment comprises about four administrations of topical steroid per day (i.e., QID) for about four weeks, followed by about three administrations of topical steroid per day (i.e., TID) for about one week, followed by about two administrations of topical steroid per day (i.e., BID) for about one week, and followed by about one administration of topical steroid per day (i.e., QD) for about one week; timing starting at about one week prior to administration of the unit dose of rAAV particles. In some embodiments, the topical steroid treatment comprises about four administrations of topical steroid per day (i.e., QID) for about 28 days, followed by about three administrations of topical steroid per day (i.e., TID) for about 7 days, followed by about two administrations of topical steroid per day (i.e., BID) for about 7 days, and followed by about one administration of topical steroid per day (i.e., QD) for about 7 days; timing starting at about 7 days prior to administration of the unit dose of rAAV particles. In some embodiments, the topical steroid treatment comprises about four administrations of topical steroid per day (i.e., QID) on Day 1 to about Day 28, followed by about three administrations of topical steroid per day (i.e., TID) on about Day 29 to about Day 35, followed by about two administrations of topical steroid per day (i.e., BID) on about Day 36 to about Day 42, and followed by about one administration of topical steroid per day (i.e., QD) on about Day 43 to about Day 49; timing starting at Day 1. In some embodiments, the topical steroid treatment is continued if inflammation is present.

In some embodiments, the methods of treatment provided herein comprise administering an anti-VEGF agent (e.g., an aflibercept IVT injection) to one eye of the individual prior to administration of the unit dose of rAAV particles to the one eye of the individual. In some embodiments, the anti-VEGF agent is administered about 7 days or about 1 week prior to administration of the unit dose of rAAV particles. In some embodiments, the anti-VEGF agent is administered on about Day 1 and the unit dose of rAAV particles is administered on about Day 8. In some embodiments, the topical steroid treatment comprises a 7-week topical steroid treatment, e.g., 0.05% difluprednate. In some embodiments, the topical steroid treatment comprises about four administrations of topical steroid per day (i.e., QID) for about four weeks, followed by about three administrations of topical steroid per day (i.e., TID) for about one week, followed by about two administrations of topical steroid per day (i.e., BID) for about one week, and followed by about one administration of topical steroid per day (i.e., QD) for about one week; timing starting with and following administration of the anti-VEGF agent. In some embodiments, the topical steroid treatment comprises about four administrations of topical steroid per day (i.e., QID) for about 28 days, followed by about three administrations of topical steroid per day (i.e., TID) for about 7 days, followed by about two administrations of topical steroid per day (i.e., BID) for about 7 days, and followed by about one administration of topical steroid per day (i.e., QD) for about 7 days; timing starting with and following administration of the anti-VEGF agent. In some embodiments, the topical steroid treatment comprises about four administrations of topical steroid per day (i.e., QID) on Day 1 to about Day 28, followed by about three administrations of topical steroid per day (i.e., TID) on about Day 29 to about Day 35, followed by about two administrations of topical steroid per day (i.e., BID) on about Day 36 to about Day 42, and followed by about one administration of topical steroid per day (i.e., QD) on about Day 43 to about Day 49; timing starting at Day 1. In some embodiments, the topical steroid treatment is continued if inflammation is present.

In some embodiments, the topical steroid treatment comprises a 4-month topical steroid treatment, e.g., 0.05% difluprednate. In some embodiments, the topical steroid treatment comprises about four administrations of topical steroid per day (i.e., QID) for about one month, followed by about three administrations of topical steroid per day (i.e., TID) for about one month, followed by about two administrations of topical steroid per day (i.e., BID) for about one month, and followed by about one administration of topical steroid per day (i.e., QD) for about one month; timing starting at about one week prior to administration of the unit dose of rAAV particles. In some embodiments, the topical steroid treatment comprises about four administrations of topical steroid per day (i.e., QID) for about 30 days, followed by about three administrations of topical steroid per day (i.e., TID) for about 30 days, followed by about two administrations of topical steroid per day (i.e., BID) for about 30 days, and followed by about one administration of topical steroid per day (i.e., QD) for about 30 days; timing starting at about 7 days prior to administration of the unit dose of rAAV particles. In some embodiments, the topical steroid treatment comprises about four administrations of topical steroid per day (i.e., QID) on Day 1 to about Day 30, followed by about three administrations of topical steroid per day (i.e., TID) on about Day 31 to about Day 60, followed by about two administrations of topical steroid per day (i.e., BID) on about Day 61 to about Day 90, and followed by about one administration of topical steroid per day (i.e., QD) on about Day 91 to about Day 120; timing starting at Day 1. In some embodiments, the topical steroid treatment is continued if inflammation is present.

In some embodiments, the methods of treatment provided herein comprise administering an anti-VEGF agent (e.g., an aflibercept IVT injection) to one eye of the individual prior to administration of the unit dose of rAAV particles to the one eye of the individual. In some embodiments, the anti-VEGF agent is administered about 7 days or about 1 week prior to administration of the unit dose of rAAV particles. In some embodiments, the anti-VEGF agent is administered on about Day 1 and the unit dose of rAAV particles is administered on about Day 8. In some embodiments, the topical steroid treatment comprises a 4-month topical steroid treatment, e.g., 0.05% difluprednate. In some embodiments, the topical steroid treatment comprises about four administrations of topical steroid per day (i.e., QID) for about one month, followed by about three administrations of topical steroid per day (i.e., TID) for about one month, followed by about two administrations of topical steroid per day (i.e., BID) for about one month, and followed by about one administration of topical steroid per day (i.e., QD) for about one month; timing starting with and following administration of the anti-VEGF agent. In some embodiments, the topical steroid treatment comprises about four administrations of topical steroid per day (i.e., QID) for about 30 days, followed by about three administrations of topical steroid per day (i.e., TID) for about 30 days, followed by about two administrations of topical steroid per day (i.e., BID) for about 30 days, and followed by about one administration of topical steroid per day (i.e., QD) for about 30 days; timing starting with and following administration of the anti-VEGF agent. In some embodiments, the topical steroid treatment comprises about four administrations of topical steroid per day (i.e., QID) on Day 1 to about Day 30, followed by about three administrations of topical steroid per day (i.e., TID) on about Day 31 to about Day 60, followed by about two administrations of topical steroid per day (i.e., BID) on about Day 61 to about Day 90, and followed by about one administration of topical steroid per day (i.e., QD) on about Day 91 to about Day 120; timing starting at Day 1. In some embodiments, the topical steroid treatment is continued if inflammation is present.

### Vectors for Delivering Transgenes to Target Cells

In some embodiments, the recombinant adeno-associated virus (rAAV) particles comprise a recombinant viral vector derived from adeno-associated virus (AAV) that has been altered so that it is replication-defective in the subject (e.g., a human or a non-human primate). In some embodiments, the adeno-associated virus (AAV) is a recombinant AAV (rAAV).

AAV or rAAV are small non-enveloped single-stranded DNA viruses. rAAVs are non-pathogenic human parvoviruses and can be made to be dependent on helper viruses, including adenovirus, herpes simplex virus, vaccinia virus and CMV, for replication.

Exposure to wild type (wt) AAV is not associated or known to cause any human pathologies and is common in the general population, making AAV or rAAV a suitable delivery system for gene therapy. AAV and rAAV used for gene therapy for delivery of an anti-VEGF agent, e.g., aflibercept, can be of any serotype. In some embodiments, the methods of the disclosure provide for use of any suitable AAV serotype, including AAV1, AAV2, AAV2.5, AAV3, AAV4, AAV5, AAV6, AAV7, AAV8, AAV9, AAV10, AAV11, AAV12, rh10, AAV-DJ, and any hybrid or chimeric AAV thereof. In some embodiments, the serotype used is based on tropism of the virus, or infectivity of a target cell of interest. In some embodiments, several AAV vectors may be generated to allow selection of the most optimal serotype for use with an anti-VEGF agent transgene (e.g., aflibercept transgene).

In some embodiments, the methods of the present disclosure provide for the use of pseudotyped AAV. Pseudotyped AAV particles comprise AAV genome inverted terminal repeats (ITRs) of one AAV serotype enbcapsidated by an AAV capsid of another AAV serotype. Typically, pseudotyped AAV is designated as “AAV #/#”, where the first “#” indicates the AAV ITR serotype and the second “#” indicates capsid serotype. For example, an AAV particle comprising AAV2 ITRs and an AAV1 capsid would be designated “AAV2/1”.

In some embodiments, the rAAV particles comprise a nucleic acid, e.g., a heterologous nucleic acid. In some embodiments, the nucleic acid encodes a transgene, e.g., an anti-VEGF agent (e.g., aflibercept). In some embodiments, the encoded transgene, e.g., anti-VEGF agent, is under the transcriptional control of a promoter that initiates transcription of the nucleic acid. In some embodiments, the promoter is a “ubiquitous” promoter. In some embodiments, the promoter is a “strong” or constitutively active promoter, e.g., a cytomegalovirus (CMV) promoter, an elongation factor 1 alpha (EFla) promoter, a glyceraldehyde 3-phosphate dehydrogenase (GAPDH) promoter, or a connexin36 (or “Cx36”) promoter. In some embodiments, the promoter is a tissue-specific promoter that is activated in specific tissues or cells, such as retinal cells, to reduce potential toxicity or undesirable effects to non-targeted cells. In some aspects, several AAV vectors may be generated to allow selection of the most optimal serotype and promoter for use with the anti-VEGF agent transgene (e.g., aflibercept transgene). In some embodiments, the nucleic acid is flanked by AAV inverted terminal repeats (ITRs). In some embodiments, the nucleic acid is flanked by AAV2 ITRs.

In some embodiments, the AAV vector comprises a polynucleotide cassette for enhanced expression of a transgene (e.g., an anti-VEGF agent such as aflibercept) in a target cell (e.g., a retinal cell). In some embodiments, the polynucleotide cassette comprises in 5′ to 3′ order: (a) a first enhancer region comprising a CMV sequence (SEQ ID NO: 22); (b) a promoter region, comprising a CMV sequence (SEQ ID NO: 23); (c) a 5′UTR region comprising, in 5′ to 3′ order, TPL and eMLP sequences (SEQ ID NO: 24 and SEQ ID NO: 25, respectively); (d) a coding sequence encoding a peptide or polypeptide (e.g., an anti-VEGF agent such as aflibercept); (e) a second enhancer region comprising a full EES sequence (SEQ ID NO: 26); and (f) a HGH polyadenylation site (SEQ ID NO: 27). In certain of these embodiments, the polynucleotide cassette comprises one or more sequences selected from SEQ ID NOs: 28-32, or a sequence with at least 85% identity thereto. In certain of these embodiments, the 5′ arm of the polynucleotide cassette comprises or consists of SEQ ID NO: 33 or a sequence with at least 85% identity thereto. In certain of these embodiments, the 3′ arm of the polynucleotide cassette comprises or consists of SEQ ID NO: 34 or a sequence with at least 85% identity thereto. The nucleic acid sequences of SEQ ID NOs: 22-34 are provided below:

(SEQ ID NO: 22)
ACTTACGGTA AATGGCCCGC CTGGCTGACC GCCCAACGAC CCCCGCCCAT TGACGTCAAT
AATGACGTAT GTTCCCATAG TAACGCCAAT AGGGACTTTC CATTGACGTC AATGGGTGGA
GTATTTACGG TAAACTGCCC ACTTGGCAGT ACATCAAGTG TATCATATGC CAAGTCCGCC
CCCTATTGAC GTCAATGACG GTAAATGGCC CGCCTGGCAT TATGCCCAGT ACATGACCTT
ACGGGACTTT CCTACTTGGC AGTACATCTA CGTATTAGTC ATCGCTATTA CCA
(SEQ ID NO: 23)
TGCTGATGCG GTTTTGGCAG TACACCAATG GGCGTGGATA GCGGTTTGAC TCACGGGGAT
TTCCAAGTCT CCACCCCATT GACGTCAATG GGAGTTTGTT TTGGCACCAA AATCAACGGG
ACTTTCCAAA ATGTCGTAAT AACCCCGCCC CGTTGACGCA AATGGGCGGT AGGCGTGTAC
GGTGGGAGGT CTATATAAGC AGAGCTCGTT TAGTGAACCG
(SEQ ID NO: 24)
CTCACTCTCT TCCGCATCGC TGTCTGCGAG GGCCAGCTGT TGGGCTCGCG GTTGAGGACA
AACTCTTCGC GGTCTTTCCA GTACTCTTGG ATCGGAAACC CGTCGGCCTC CGAACGGTAC
TCCGCCACCG AGGGACCTGA GCGAGTCCGC ATCGACCGGA TCGGAAAACC TCTCGAGAAA
GGCGTCTAAC CAGTCACAGT CGCAAGGTAG GCTGAGCACC GTGGCGGGCG GCAGCGGGTG
GCGGTCGGGG TTGTTTCTGG CGGAGGTGCT GCTGATGATG TAATTAAAGT AGGCGGTCTT
GAGACGGCGG ATGGTCGA
(SEQ ID NO: 25)
CCAGCTGTTG GGGTGAGTAC TCCCTCTCAA AAGCGGGCAT TACTTCTGCG CTAAGATTGT
CAGTTTCCAA AAACGAGGAG GATTTGATAT TCACCTGGCC CG
(SEQ ID NO: 26)
CTGTTCTCAT CACATCATAT CAAGGTTATA TACCATCAAT ATTGCCACAG ATGTTACTTA
GCCTTTTAAT ATTTCTCTAA TTTAGTGTAT ATGCAATGAT AGTTCTCTGA TTTCTGAGAT
TGAGTTTCTC ATGTGTAATG ATTATTTAGA GTTTCTCTTT CATCTGTTCA AATTTTTGTC
TAGTTTTATT TTTTACTGAT TTGTAAGACT TCTTTTTATA ATCTGCATAT TACAATTCTC
TTTACTGGGG TGTTGCAAAT ATTTTCTGTC ATTCTATGGC CTGACTTTTC TTAATGGTTT
TTTAATTTTA AAAATAAGTC TTAATATTCA TGCAATCTAA TTAACAATCT TTTCTTTGTG
GTTAGGACTT TGAGTCATAA GAAATTTTTC TCTACACTGA AGTCATGATG GCATGCTTCT
ATATTATTTT CTAAAAGATT TAAAGTTTTG CCTTCTCCAT TTAGACTTAT AATTCACTGG
AATTTTTTTG TGTGTATGGT ATGACATATG GGTTCCCTTT TATTTTTTAC ATATAAATAT
ATTTCCCTGT TTTTCTAAAA AAGAAAAAGA TCATCATTTT CCCATTGTAA AATGCCATAT
TTTTTTCATA GGTCACTTAC ATATATCAAT GGGTCTGTTT CTGAGCTCTA CTCTATTTTA
TCAGCCTCAC TGTCTATCCC CACACATCTC ATGCTTTGCT CTAAATCTTG ATATTTAGTG
GAACATTCTT TCCCATTTTG TTCTACAAGA ATATTTTTGT TATTGTCTTT GGGCTTTCTA
TATACATTTT GAAATGAGGT TGACAAGTTA
(SEQ ID NO: 27)
CTGCCCGGGT GGCATCCCTG TGACCCCTCC CCAGTGCCTC TCCTGGCCCT GGAAGTTGCC
ACTCCAGTGC CCACCAGCCT TGTCCTAATA AAATTAAGTT GCATCATTTT GTCTGACTAG
GTGTCCTTCT ATAATATTAT GGGGTGGAGG GGGGTGGTAT GGAGCAAGGG GCCCAAGTTG
GGAAGAAACC TGTAGGGCCT GC
(SEQ ID NO: 28)
AGGCGGTCTT GAGACGGCGG ATGGTCGAGG TGAGGTGTGG CAGGCTTGAG ATCCAGCTGT
TGGGGTGA
(SEQ ID NO: 29)
CGCTGTTTTG ACCTCCATAG TGGACACCGG GACCGATCCA GCCTCCGCGT CTCAGGGGAG
ATCTCGTTTA GTGAACCGTC AGATCCTCAC TCTCTTCCGC ATCGCTGTCT GCGAGGGCCA
GCTGTTGGG
(SEQ OD NO: 30)
TTGATATTCA CCTGGCCCGA TCTGGCCATA CACTTG
(SEQ ID NO: 31)
CCCAGGTCCA AGTTTAAACG CC
(SEQ ID NO: 32)
TCTTTGGGCT TTCTATATAC ATTTTGAAAT GAGGTTGACA AGTTACCTAG GAAAACTGTC
TTCCTGCCCG GGTGGCA
(SEQ ID NO: 33)
CTCTGGAGAC GACTTACGGT AAATGGCCCG CCTGGCTGAC CGCCCAACGA CCCCCGCCCA
TTGACGTCAA TAATGACGTA TGTTCCCATA GTAACGCCAA TAGGGACTTT CCATTGACGT
CAATGGGTGG AGTATTTACG GTAAACTGCC CACTTGGCAG TACATCAAGT GTATCATATG
CCAAGTCCGC CCCCTATTGA CGTCAATGAC GGTAAATGGC CCGCCTGGCA TTATGCCCAG
TACATGACCT TACGGGACTT TCCTACTTGG CAGTACATCT ACGTATTAGT CATCGCTATT
ACCATGCTGA TGCGGTTTTG GCAGTACACC AATGGGCGTG GATAGCGGTT TGACTCACGG
GGATTTCCAA GTCTCCACCC CATTGACGTC AATGGGAGTT TGTTTTGGCA CCAAAATCAA
CGGGACTTTC CAAAATGTCG TAATAACCCC GCCCCGTTGA CGCAAATGGG CGGTAGGCGT
GTACGGTGGG AGGTCTATAT AAGCAGAGCT CGTTTAGTGA ACCGTCAGAT CGCCTGGAGA
GGCCATCCAC GCTGTTTTGA CCTCCATAGT GGACACCGGG ACCGATCCAG CCTCCGCGTC
TCAGGGGAGA TCTCGTTTAG TGAACCGTCA GATCCTCACT CTCTTCCGCA TCGCTGTCTG
CGAGGGCCAG CTGTTGGGCT CGCGGTTGAG GACAAACTCT TCGCGGTCTT TCCAGTACTC
TTGGATCGGA AACCCGTCGG CCTCCGAACG GTACTCCGCC ACCGAGGGAC CTGAGCGAGT
CCGCATCGAC CGGATCGGAA AACCTCTCGA GAAAGGCGTC TAACCAGTCA CAGTCGCAAG
GTAGGCTGAG CACCGTGGCG GGCGGCAGCG GGTGGCGGTC GGGGTTGTTT CTGGCGGAGG
TGCTGCTGAT GATGTAATTA AAGTAGGCGG TCTTGAGACG GCGGATGGTC GAGGTGAGGT
GTGGCAGGCT TGAGATCCAG CTGTTGGGGT GAGTACTCCC TCTCAAAAGC GGGCATTACT
TCTGCGCTAA GATTGTCAGT TTCCAAAAAC GAGGAGGATT TGATATTCAC CTGGCCCGAT
CTGGCCATAC ACTTGAGTGA CAATGACATC CACTTTGCCT TTCTCTCCAC AGGTGTCCAC
TCCCAGGTCC AAGTTTAAAC GCCGCCACCA TG
(SEQ ID NO: 34)
ACTGTTCTCA TCACATCATA TCAAGGTTAT ATACCATCAA TATTGCCACA GATGTTACTT
AGCCTTTTAA TATTTCTCTA ATTTAGTGTA TATGCAATGA TAGTTCTCTG ATTTCTGAGA
TTGAGTTTCT CATGTGTAAT GATTATTTAG AGTTTCTCTT TCATCTGTTC AAATTTTTGT
CTAGTTTTAT TTTTTACTGA TTTGTAAGAC TTCTTTTTAT AATCTGCATA TTACAATTCT
CTTTACTGGG GTGTTGCAAA TATTTTCTGT CATTCTATGG CCTGACTTTT CTTAATGGTT
TTTTAATTTT AAAAATAAGT CTTAATATTC ATGCAATCTA ATTAACAATC TTTTCTTTGT
GGTTAGGACT TTGAGTCATA AGAAATTTTT CTCTACACTG AAGTCATGAT GGCATGCTTC
TATATTATTT TCTAAAAGAT TTAAAGTTTT GCCTTCTCCA TTTAGACTTA TAATTCACTG
GAATTTTTTT GTGTGTATGG TATGACATAT GGGTTCCCTT TTATTTTTTA CATATAAATA
TATTTCCCTG TTTTTCTAAA AAAGAAAAAG ATCATCATTT TCCCATTGTA AAATGCCATA
TTTTTTTCAT AGGTCACTTA CATATATCAA TGGGTCTGTT TCTGAGCTCT ACTCTATTTT
ATCAGCCTCA CTGTCTATCC CCACACATCT CATGCTTTGC TCTAAATCTT GATATTTAGT
GGAACATTCT TTCCCATTTT GTTCTACAAG AATATTTTTG TTATTGTCTT TGGGCTTTCT
ATATACATTT TGAAATGAGG TTGACAAGTT ACCTAGGAAA ACTGTCTTCC TGCCCGGGTG
GCATCCCTGT GACCCCTCCC CAGTGCCTCT CCTGGCCCTG GAAGTTGCCA CTCCAGTGCC
CACCAGCCTT GTCCTAATAA AATTAAGTTG CATCATTTTG TCTGACTAGG TGTCCTTCTA
TAATATTATG GGGTGGAGGG GGGTGGTATG GAGCAAGGGG CCCAAGTTGG GAAGAAACCT
GTAGGGCCTG CGAAGACAGT CAG

In some embodiments, the polynucleotide cassette comprises or consists of SEQ ID NO: 39 or a sequence with at least 85% identity thereto.

(SEQ ID NO: 39)
gcgcgctcgctcgctcactgaggccgcccgggcaaagcccgggcgtcgggcgacctttggtcgcccggcctca
gtgagcgagcgagcgcgcagagagggagtggccaactccatcactaggggttccttgtagttaatgattaacc
cgccatgctacttatctacgtactctggagacgacttacggtaaatggcccgcctggctgaccgcccaacgac
ccccgcccattgacgtcaataatgacgtatgttcccatagtaacgccaatagggactttccattgacgtcaat
gggtggagtatttacggtaaactgcccacttggcagtacatcaagtgtatcatatgccaagtccgccccctat
tgacgtcaatgacggtaaatggcccgcctggcattatgcccagtacatgaccttacgggactttcctacttgg
cagtacatctacgtattagtcatcgctattaccatgctgatgcggttttggcagtacaccaatgggcgtggat
agcggtttgactcacggggatttccaagtctccaccccattgacgtcaatgggagtttgttttggcaccaaaa
tcaacgggactttccaaaatgtcgtaataaccccgccccgttgacgcaaatgggcggtaggcgtgtacggtgg
gaggtctatataagcagagctcgtttagtgaaccgtcagatcgcctggagaggccatccacgctgttttgacc
tccatagtggacaccgggaccgatccagcctccgcgtctcaggggagatctcgtttagtgaaccgtcagatcc
tcactctcttccgcatcgctgtctgcgagggccagctgttgggctcgcggttgaggacaaactcttcgcggtc
tttccagtactcttggatcggaaacccgtcggcctccgaacggtactccgccaccgagggacctgagcgagtc
cgcatcgaccggatcggaaaacctctcgagaaaggcgtctaaccagtcacagtcgcaaggtaggctgagcacc
gtggcgggcggcagcgggtggcggtcggggttgtttctggcggaggtgctgctgatgatgtaattaaagtagg
cggtcttgagacggcggatggtcgaggtgaggtgtggcaggcttgagatccagctgttggggtgagtactccc
tctcaaaagcgggcattacttctgcgctaagattgtcagtttccaaaaacgaggaggatttgatattcacctg
gcccgatctggccatacacttgagtgacaatgacatccactttgcctttctctccacaggtgtccactcccag
gtccaagtttaaacgccgccaccatggtgtcatactgggatactggagtcttgctttgtgccctgctgtcctg
cctcctcctgactggctccagctcgggctcagataccggtcgccccttcgtggagatgtactccgagatcccg
gaaattatccacatgactgaggggcgcgaacttgtgatcccctgccgggtcaccagcccgaacattactgtga
ctttgaagaagttccccctggacaccctgattccggatgggaagagaattatctgggattcacggaagggatt
catcatcagcaacgcgacctacaaggaaattggcctcctcacttgcgaagccactgtgaacggacacttgtac
aagaccaactacctgacccaccgccagaccaacaccatcatcgacgtcgtcctgtccccttcgcacgggatcg
agctctcggtgggagagaagttggtgcttaactgcaccgcccggacggaactgaatgtgggaatcgacttcaa
ctgggaatacccgtccagcaagcatcagcataagaagctggtgaaccgggacctcaagactcagtccggcagc
gaaatgaagaagttcctgtcgaccctcactattgacggagtgaccagatccgaccagggcctctacacttgcg
ccgcttccagcggactcatgaccaagaagaacagcactttcgtgagggtgcatgagaaggacaagacccacac
gtgtccgccgtgcccagccccagagctgctgggaggcccttccgtgttcctgtttccgcccaagccaaaggat
accctgatgatctcaaggacccctgaggtcacatgcgtcgtggtggatgtgtcgcacgaggaccctgaagtca
aattcaattggtatgtggacggagtggaagtccacaacgcgaaaaccaagccgagagaagaacagtacaattc
cacctaccgggtggtgtcggtgctgactgtgctgcaccaggactggctcaacggaaaggagtacaagtgcaag
gtgtccaacaaggctctgcccgcacctattgaaaagaccatctccaaggccaagggtcaacctcgcgagcctc
aggtgtacactctgcctccaagccgggacgaactgactaagaaccaagtctctctgacctgtttggtgaaggg
cttctacccgtcagacatcgcagtggagtgggagtcaaacggtcagccggagaacaactacaaaacaaccccc
cccgtgctggactccgacggctccttcttcctgtactccaagcttaccgtggataagagccgctggcaacagg
gcaacgtgttttcctgctccgtcatgcacgaagccctgcacaaccattatacccagaagtccctgtcgctgtc
ccccgggaaatagtgactgttctcatcacatcatatcaaggttatataccatcaatattgccacagatgttac
ttagccttttaatatttctctaatttagtgtatatgcaatgatagttctctgatttctgagattgagtttctc
atgtgtaatgattatttagagtttctctttcatctgttcaaatttttgtctagttttattttttactgatttg
taagacttctttttataatctgcatattacaattctctttactggggtgttgcaaatattttctgtcattcta
tggcctgacttttcttaatggttttttaattttaaaaataagtcttaatattcatgcaatctaattaacaatc
ttttctttgtggttaggactttgagtcataagaaatttttctctacactgaagtcatgatggcatgcttctat
attattttctaaaagatttaaagttttgccttctccatttagacttataattcactggaatttttttgtgtgt
atggtatgacatatgggttcccttttattttttacatataaatatatttccctgtttttctaaaaaagaaaaa
gatcatcattttcccattgtaaaatgccatatttttttcataggtcacttacatatatcaatgggtctgtttc
tgagctctactctattttatcagcctcactgtctatccccacacatctcatgctttgctctaaatcttgatat
ttagtggaacattctttcccattttgttctacaagaatatttttgttattgtctttgggctttctatatacat
tttgaaatgaggttgacaagttacctaggaaaactgtcttcctgcccgggtggcatccctgtgacccctcccc
agtgcctctcctggccctggaagttgccactccagtgcccaccagccttgtcctaataaaattaagttgcatc
attttgtctgactaggtgtccttctataatattatggggtggaggggggtggtatggagcaaggggcccaagt
tgggaagaaacctgtagggcctgcgtacgtagataagtagcatggcgggttaatcattaactacaaggaaccc
ctagtgatggagttggccactccctctctgcgcgctcgctcgctcactgaggccgggcgaccaaaggtcgccc
gacgcccgggctttgcccgggcggcctcagtgagcgagcgagcgcgc

SEQ ID NO: 39 shown above comprises, in the 5′ to 3′ direction, an inverted terminal repeat (ITR) of AAV serotype 2 comprising nucleotides 1-145 of SEQ ID NO: 39; a CMV promoter comprising nucleotides 180-693 of SEQ ID NO: 39; a 5′ Untranslated Region (UTR), including an Adenovirus Tripartite Leader Sequence and Synthetic Intron, and comprising nucleotides 694-1314 of SEQ ID NO: 39; a Kozak sequence comprising nucleotides 1329-1340 of SEQ ID NO: 39; a codon-optimized aflibercept cDNA sequence comprising nucleotides 1338-2714 of SEQ ID NO: 39; a 3′ UTR including a human scaffold attachment region and comprising nucleotides 2717-3527 of SEQ ID NO: 39; a human growth hormone polyadenylation/transcription stop signal comprising nucleotides 3546-3748 of SEQ ID NO: 39; and an inverted terminal repeat (ITR) of AAV serotype 2 comprising nucleotides 3772-3916 of SEQ ID NO: 39.

Additional polynucleotide cassettes for enhanced expression of a transgene (e.g., a transgene encoding an anti-VEGF agent such as aflibercept) in a target cell (such as a retinal cell) are disclosed in WO2018/170473, the contents of which related to polynucleotide cassettes for enhanced expression of a transgene in a target cell are incorporated herein by reference.

In some embodiments, the rAAV particles comprise a variant capsid protein having increased infectivity of target cells, e.g. retinal cells, are used to increase transduction of retinal cells or to increase targeting of gene delivery to retinal cells in an individual. In some embodiments, the rAAV particle comprises an amino acid modification in a capsid protein GH loop/loop IV of the AAV capsid protein. In some embodiments, the site of modification is a solvent-accessible portion of the GH loop/loop IV of the AAV capsid protein. For a description of the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. Several AAV capsid variants are known, including the 7m8 variant. In some embodiments, a rAAV particle comprises a variant AAV capsid protein that comprises an insertion of from 5 amino acids to 11 amino acids, e.g., 7 amino acid sequence, in the GH loop of a capsid protein relative to a corresponding parental AAV capsid protein, and wherein the variant capsid protein confers increased infectivity of a retinal cell compared to the infectivity of the retinal cell by an AAV particle comprising the corresponding parental or unmodified AAV capsid protein. In some embodiments, any one of the following amino acid sequences can be inserted in the GH loop of a capsid protein: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21). In some embodiments, any one of the amino acid sequences set forth in SEQ ID NOs: 1-12 and 14-21 is inserted in the solvent-exposed GH loop of VP1 capsid protein in a rAAV. Additional details regarding amino acid sequences that can be inserted into the GH loop of a capsid protein, e.g., to facilitate transduction of a nucleic acid of interest to a retinal cell following IVT injection, are provided in WO2012145601, U.S. Pat. Nos. 9,587,282, 10,202,657, and 10,214,785, the contents of which related to amino acid sequences that can be inserted into the GH loop of a capsid protein are incorporated herein by reference.

In some embodiments, the rAAV particles comprise an AAV capsid protein, e.g., an AAV2 capsid protein, that includes any one of the following amino acid sequences: LALGETTRPA (SEQ ID NO: 1); LANETITRPA (SEQ ID NO: 2), LAKAGQANNA (SEQ ID NO: 3), LAKDPKTTNA (SEQ ID NO: 4), KDTDTTR (SEQ ID NO: 5), RAGGSVG (SEQ ID NO: 6), AVDTTKF (SEQ ID NO: 7), STGKVPN (SEQ ID NO: 8), LAKDTDTTRA (SEQ ID NO: 9), LARAGGSVGA (SEQ ID NO: 10), LAAVDTTKFA (SEQ ID NO: 11), and LASTGKVPNA (SEQ ID NO: 12), LGETTRP (SEQ ID NO: 14), NETITRP (SEQ ID NO: 15), KAGQANN (SEQ ID NO: 16), KDPKTTN (SEQ ID NO: 17), KDTDTTR (SEQ ID NO: 18), RAGGSVG (SEQ ID NO: 19), AVDTTKF (SEQ ID NO: 20), and STGKVPN (SEQ ID NO: 21) inserted at the following positions: between positions 587 and 588 of the AAV2 capsid protein; between amino acids 590 and 591 of the AAV1 capsid protein; between amino acids 575 and 576 of the AAV5 capsid protein; between amino acids 590 and 591 of the AAV6 capsid protein; between amino acids 589 and 590 of the AAV7 capsid protein; between amino acids 590 and 591 of the AAV8 capsid protein; between amino acids 588 and 589 of the AAV9 capsid protein; or between amino acids 589 and 590 of the AAV10 capsid protein. In some embodiments, the rAAV particles comprise AAV2 capsid proteins comprising an amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise AAV2 capsid proteins comprising the amino acid sequence LALGETTRPA (SEQ ID NO: 1) inserted between positions 587 and 588 of the AAV2 VP1 comprising the sequence of SEQ ID NO: 13.

In some embodiments, rAAV particles comprise the 7m8 variant capsid protein from AAV2 comprising the amino acid sequence LALGETTRPA (SEQ ID NO: 1) inserted in the GH loop of the AAV2 VP1 protein between positions 587 and 588 of the AAV2 VP1. In some embodiments, the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38. In some embodiments, the rAAV particles comprise an AAV2 VP1 capsid protein comprising a GH loop that comprises an amino acid sequence having any of at least 90%, at least 91%, at least 92%, at least 93%, at least 94%, at least 95%, at least 96%, at least 97%, at least 98%, at least 99%, or 100% sequence identity to SEQ ID NO: 38.

(SEQ ID NO: 38)
FSYTFEDVPFHSSYAHSQSLDRLMNPLIDQYLYYLSRTNTPSGTTTQSRLQFSQAGASDIRDQSRNWLPGPCY
RQQRVSKTSADNNNSEYSWTGATKYHLNGRDSLVNPGPAMASHKDDEEKFFPQSGVLIFGKQGSEKTNVDIEK
VMITDEEEIRTTNPVATEQYGSVSTNLQRGNLALGETTRPARQAATADVNTQGVLPGMVWQDRDVYLQGPIWA
KIPHTDGHFHPSPLMGGFGLKHPPPQILIKN

In some embodiments, rAAV particles comprise the 7m8 variant capsid protein from AAV2 comprising the amino acid sequence LALGETTRPA (SEQ ID NO: 1) inserted between positions 587 and 588 of the AAV2 VP1. The sequence of the 7m8 variant capsid protein from AAV2 comprising the amino acid sequence LALGETTRPA (SEQ ID NO: 1) is inserted between positions 587 and 588 of the AAV2 VP1 is provided below:

(SEQ ID NO: 37)
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGAD
GVGNSSGNWHCDSTWMGDRVITTSTRTWALPTYNNHLYKQISSQSGASNDNHYFGYSTPWGYEDFNRFHCHFS
PRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLI
DQYLYYLSRTNTPSGTTTQSRLQFSQAGASDIRDQSRNWLPGPCYRQQRVSKTSADNNNSEYSWTGATKYHLN
GRDSLVNPGPAMASHKDDEEKFFPQSGVLIFGKQGSEKTNVDIEKVMITDEEEIRTTNPVATEQYGSVSTNLQ
RGNLALGETTRPARQAATADVNTQGVLPGMVWQDRDVYLQGPIWAKIPHTDGHFHPSPLMGGFGLKHPPPQIL
IKNTPVPANPSTTESAAKFASFITQYSTGQVSVEIEWELQKENSKRWNPEIQYTSNYNKSVNVDFTVDINGVY
SEPRPIGTRYLTRNL

In some embodiments, the rAAV particles comprise a capsid protein VP1 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise a capsid protein VP2 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise a capsid protein VP3 comprising the amino acid sequence LGETTRP (SEQ ID NO: 14) inserted between positions 587 and 588 of the capsid protein, wherein the amino acid residue numbering corresponds to an AAV2 VP1 capsid protein. In some embodiments, the rAAV particles comprise capsid proteins VP1, VP2, and VP3, wherein each of VP1, VP2, and VP3 comprise the amino acid sequence LGETTRP (SEQ ID NO: 14) inserted between positions 587 and 588 of the capsid protein, wherein the amino acid residue numbering corresponds to an AAV2 VP1 capsid protein.

In some embodiments, the rAAV particles comprise a capsid protein VP1 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise a capsid protein VP2 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise a capsid protein VP3 comprising the amino acid sequence LALGETTRPA (SEQ ID NO: 1) inserted between positions 587 and 588 of the capsid protein, wherein the amino acid residue numbering corresponds to an AAV2 VP1 capsid protein. In some embodiments, the rAAV particles comprise capsid proteins VP1, VP2, and VP3, wherein each of VP1, VP2, and VP3 comprise the amino acid sequence LALGETTRPA (SEQ ID NO: 1) inserted between positions 587 and 588 of the capsid protein, wherein the amino acid residue numbering corresponds to an AAV2 VP1 capsid protein.

In some embodiments, a recombinant virus and/or plasmid used to generate a rAAV virus comprises other transcriptional or regulatory elements, such as a poly A (polyadenylation) sequence, untranslated regions (UTRs), 3′ UTRs, or termination sequences. In some embodiments, more than one gene is expressed from the vector or plasmid using internal ribosome entry site (IRES) or similar element that allows co-expression of two or more proteins or create multigene, or polycistronic mRNA.

In some embodiments, the rAAV and/or plasmid used to generate the rAAV comprises one or more of the following nucleic acid elements: a first ITR sequence; a promoter sequence; an intron sequence; a first UTR sequence; a heterologous nucleic acid encoding an anti-VEGF agent (e.g., aflibercept); a second UTR sequence; a polyA sequence; and a second ITR sequence. In some embodiments, linker sequence(s) are inserted between two or more of the nucleic acid elements. In some embodiments, the heterologous nucleic acid encodes a therapeutic polypeptide, e.g., encodes aflibercept (or a functional fragment or functional variant thereof).

In some embodiments, the vector is a targeted vector, especially a targeted rAAV (e.g., AAV2.7m8) that shows higher infectivity of a specific cell, such as a retinal cell (e.g., a photoreceptor, a retinal ganglion cell, a Müller cell, a bipolar cell, an amacrine cell, a horizontal cell, or a retinal pigmented epithelium cell). Viral vectors for use in the disclosure can include those that exhibit low toxicity and/or low immunogenicity in an individual and expresses therapeutically effective quantities of the anti-VEGF agent (e.g., aflibercept) in an individual, e.g., a human. Any suitable method known in the art can be used in the biochemical purification of recombinant viruses (e.g., rAAV), e.g., for the preparation of pharmaceutical compositions described elsewhere herein. Recombinant AAV viruses can be harvested directly from cells, or from the culture media comprising cells. Virus can be purified using various biochemical means, such as gel filtration, filtration, chromatography, affinity purification, gradient ultracentrifugation, or size exclusion methods. In some embodiments, the virus is lyophilized.

In some embodiments, the rAAV particles comprise a 7m8 variant capsid protein, e.g., rAAV2.7m8, and a nucleic acid sequence that encodes an anti-VEGF agent (e.g., aflibercept, or a functional fragment or functional variant thereof). In some embodiments, the rAAV particles (e.g., the 7m8 variant) have an increase in retinal cell infectivity of any of at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in infectivity of retinal cells is an increase of any of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10%, as compared to an AAV particle comprising the corresponding parental or unmodified AAV capsid protein.

In some embodiments, the increase in retinal cell infectivity of a rAAV variant, e.g., rAAV2.7m8, is any of at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental or unmodified AAV capsid protein. In some embodiments, the increase in infectivity is any of at least 2-fold, at least 3-fold, at least 4-fold, at least 5-fold, at least 6-fold, at least 7-fold, at least 8-fold, at least 9-fold, or at least 10-fold, as compared to an AAV particle comprising the corresponding parental AAV capsid protein. In some embodiments, the increase in infectivity is any of at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in retinal cell infectivity of a rAAV variant, e.g., rAAV2.7m8, is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold, as compared to an AAV particle comprising the corresponding parental or unmodified AAV capsid protein.

In some embodiments, the increase in retinal cell infectivity is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold, as compared to an AAV particle comprising the corresponding parental or unmodified AAV capsid protein.

In some embodiments, an amino acid modification of a capsid protein described herein can confer an increase in an ability to cross an internal limiting membrane (ILM) in an eye of an individual, e.g., a human, as compared to the ability of an AAV particle comprising the corresponding parental or unmodified AAV capsid protein to cross the ILM in the eye of the subject. In some embodiments, the increase in the ability to cross the ILM of a rAAV variant, e.g., rAAV2.7m8, is an increase of any of at least 5%, at least 10%, at least 20%, at least 30%, at least 40%, at least 50%, at least 60%, at least 70%, at least 80%, at least 90%, or at least 100% as compared to an AAV particle comprising the corresponding parental or unmodified AAV capsid protein. In some embodiments, the increase in the ability to cross the ILM is an increase of between 5% to 100%, between 5% to 95%, between 5% to 90%, between 5% to 85%, between 5% to 80%, between 5% to 75%, between 5% to 70%, between 5% to 65%, between 5% to 60%, between 5% to 55%, between 5% to 50%, between 5% to 45%, between 5% to 40%, between 5% to 35%, between 5% to 30%, between 5% to 25%, between 5% to 20%, between 5% to 15%, or between 5% to 10%, as compared to the parental or unmodified AAV capsid protein.

In some embodiments, the increase in the ability to cross the ILM of a rAAV variant, e.g., rAAV2.7m8, is any of at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2-fold compared to an AAV particle comprising the corresponding parental AAV capsid protein. In some embodiments, the increase in the ability to cross the ILM is any of at least 2-fold, at least 3-fold, at least 4-fold, at least 5-fold, at least 6-fold, at least 7-fold, at least 8-fold, at least 9-fold, or at least 10-fold, as compared to an AAV particle comprising the corresponding parental AAV capsid protein. In some embodiments, the increase in the ability to cross the ILM is any of at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, or at least 100-fold compared to an AAV particle comprising the corresponding parental or unmodified AAV capsid protein.

In some embodiments, the increase in the ability to cross the ILM of a rAAV variant, e.g., rAAV2.7m8, is between 10-fold to 100-fold, between 10-fold to 95-fold, between 10-fold to 90-fold, between 10-fold to 85-fold, between 10-fold to 80-fold, between 10-fold to 75-fold, between 10-fold to 70-fold, between 10-fold to 65-fold, between 10-fold to 60-fold, between 10-fold to 55-fold, between 10-fold to 50-fold, between 10-fold to 45-fold, between 10-fold to 40-fold, between 10-fold to 35-fold, between 10-fold to 30-fold, between 10-fold to 25-fold, between 10-fold to 20-fold, or between 10-fold to 15-fold as compared to an AAV particle comprising the corresponding parental or unmodified AAV capsid protein.

In some embodiments, the increase in the ability to cross the ILM of a rAAV variant, e.g., rAAV2.7m8, is between 2-fold to 20-fold, between 2-fold to 19-fold, between 2-fold to 18-fold, between 2-fold to 17-fold, between 2-fold to 16-fold, between 2-fold to 15-fold, between 2-fold to 14-fold, between 2-fold to 13-fold, between 2-fold to 12-fold, between 2-fold to 11-fold, between 2-fold to 10-fold, between 2-fold to 9-fold, between 2-fold to 8-fold, between 2-fold to 7-fold, between 2-fold to 6-fold, between 2-fold to 5-fold, between 2-fold to 4-fold, or between 2-fold to 3-fold, as compared to an AAV particle comprising the corresponding parental or unmodified AAV capsid protein.

In some embodiments, rAAV.7m8 comprising nucleic acid encoding aflibercept is used for gene therapy. In some embodiments, AAV2 or rAAV2 is used to deliver a nucleic acid sequence encoding an anti-VEGF agent (e.g., aflibercept) into an eye or retinal cells of a subject via intravitreal or subretinal injection. In some embodiments, AAV2 or rAAV2 is used to deliver a nucleic acid sequence encoding an anti-VEGF agent (e.g., aflibercept) into an eye or retinal cells of a subject via intravitreal injection. In some embodiments, rAAV2.7m8 is used to deliver the nucleic acid sequence of the anti-VEGF agent (e.g., aflibercept) into the retinal cells of a subject. In some embodiments, the heterologous nucleic acid (e.g., a nucleic acid that encodes an anti-VEGF agent such as aflibercept) integrates into the target cell genome (e.g., retinal cell genome), resulting in long-term expression of, e.g., the anti-VEGF agent (such as aflibercept), in the target cell. In some embodiments, the viral vector delivers a plasmid or other extrachromosomal genetic element that comprises the heterologous nucleic acid (e.g., a nucleic acid that encodes an anti-VEGF agent such as aflibercept) to the target cell (e.g., retinal cell).

In some embodiments, the rAAV particles comprise a nucleic acid encoding a polypeptide comprising an amino acid sequence with any of at least about 95%, at least about 96%, at least about 97%, at least about 98%, at least about 99%, or at least about 100% identity to the amino acid sequence of SEQ ID NO: 35 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the rAAV particles comprise a nucleic acid encoding a polypeptide comprising the amino acid sequence of SEQ ID NO: 35 and flanked by AAV2 inverted terminal repeats (ITRs). In some embodiments, the rAAV particles comprise a nucleic acid encoding a polypeptide comprising the amino acid sequence of SEQ ID NO: 35. In some embodiments, the rAAV particles comprise a nucleic acid encoding aflibercept and flanked by AAV2 inverted terminal repeats (ITRs). The sequence of SEQ ID NO: 35 is provided below:

(SEQ ID NO: 35)
SDTGRPFVEMYSEIPEIIHMTEGRELVIPCRVTSPNITVTLKKFPLDTLIPDGKRIIWDSRKGFIISNATYKE
IGLLTCEATVNGHLYKTNYLTHRQTNTIIDVVLSPSHGIELSVGEKLVLNCTARTELNVGIDFNWEYPSSKHQ
HKKLVNRDLKTQSGSEMKKFLSTLTIDGVTRSDQGLYTCAASSGLMTKKNSTFVRVHEKDKTHTCPPCPAPEL
LGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPG

In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or at least about 100% sequence homology to the nucleic acid sequence of SEQ ID NO: 36, and wherein the nucleic acid is flanked by AAV2 inverted terminal repeats (ITRs). In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or at least about 100% sequence homology to the nucleic acid sequence of aflibercept (e.g., SEQ ID NO: 36), and wherein the nucleic acid is flanked by AAV2 inverted terminal repeats (ITRs). In some embodiments, the nucleic acid sequence of aflibercept is derived from its amino acid sequence. In some embodiments, the nucleic acid sequence of aflibercept is codon optimized to improve its expression in a subject. In some embodiments, the rAAV particles comprise a nucleic acid with any of at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.9%, or 100% sequence homology to the nucleic acid sequence of SEQ ID NO: 40, and wherein the nucleic acid is flanked by AAV2 inverted terminal repeats (ITRs). In some embodiments, the rAAV particles comprise a nucleic acid comprising the nucleic acid sequence of SEQ ID NO: 40. In some embodiments, the rAAV particles comprise a nucleic acid comprising the nucleic acid sequence of SEQ ID NO: 40, and wherein the nucleic acid is flanked by AAV2 inverted terminal repeats (ITRs).

(SEQ ID NO: 40)
atggtgtcatactgggatactggagtcttgctttgtgccctgctgtcctgcctcctcctgactggctccagct
cgggctcagataccggtcgccccttcgtggagatgtactccgagatcccggaaattatccacatgactgaggg
gcgcgaacttgtgatcccctgccgggtcaccagcccgaacattactgtgactttgaagaagttccccctggac
accctgattccggatgggaagagaattatctgggattcacggaagggattcatcatcagcaacgcgacctaca
aggaaattggcctcctcacttgcgaagccactgtgaacggacacttgtacaagaccaactacctgacccaccg
ccagaccaacaccatcatcgacgtcgtcctgtccccttcgcacgggatcgagctctcggtgggagagaagttg
gtgcttaactgcaccgcccggacggaactgaatgtgggaatcgacttcaactgggaatacccgtccagcaagc
atcagcataagaagctggtgaaccgggacctcaagactcagtccggcagcgaaatgaagaagttcctgtcgac
cctcactattgacggagtgaccagatccgaccagggcctctacacttgcgccgcttccagcggactcatgacc
aagaagaacagcactttcgtgagggtgcatgagaaggacaagacccacacgtgtccgccgtgcccagccccag
agctgctgggaggcccttccgtgttcctgtttccgcccaagccaaaggataccctgatgatctcaaggacccc
tgaggtcacatgcgtcgtggtggatgtgtcgcacgaggaccctgaagtcaaattcaattggtatgtggacgga
gtggaagtccacaacgcgaaaaccaagccgagagaagaacagtacaattccacctaccgggtggtgtcggtgc
tgactgtgctgcaccaggactggctcaacggaaaggagtacaagtgcaaggtgtccaacaaggctctgcccgc
acctattgaaaagaccatctccaaggccaagggtcaacctcgcgagcctcaggtgtacactctgcctccaagc
cgggacgaactgactaagaaccaagtctctctgacctgtttggtgaagggcttctacccgtcagacatcgcag
tggagtgggagtcaaacggtcagccggagaacaactacaaaacaaccccccccgtgctggactccgacggctc
cttcttcctgtactccaagcttaccgtggataagagccgctggcaacagggcaacgtgttttcctgctccgtc
atgcacgaagccctgcacaaccattatacccagaagtccctgtcgctgtcccccgggaaatag

In some embodiments, the rAAV particles comprise a nucleic acid encoding a polypeptide comprising an amino acid sequence with any of at least about 95%, at least about 96%, at least about 97%, at least about 98%, at least about 99%, or 100% identity to the amino acid sequence of SEQ ID NO: 41 and flanked by AAV2 inverted terminal repeats (ITRs). In some embodiments, the rAAV particles comprise a nucleic acid encoding a polypeptide comprising an amino acid sequence with at least about 95% identity to the amino acid sequence of SEQ ID NO: 41 and flanked by AAV2 inverted terminal repeats (ITRs). In some embodiments, the rAAV particles comprise a nucleic acid encoding a polypeptide comprising the amino acid sequence of SEQ ID NO: 41 and flanked by AAV2 inverted terminal repeats (ITRs). In some embodiments, the rAAV particles comprise a nucleic acid encoding a polypeptide comprising the amino acid sequence of SEQ ID NO: 41.

(SEQ ID NO: 41)
MVSYWDTGVLLCALLSCLLLTGSSSGSDTGRPFVEMYSEIPEIIHMTEGRELVIPCRVTSPNITVTLKKFPLD
TLIPDGKRIIWDSRKGFIISNATYKEIGLLTCEATVNGHLYKTNYLTHRQTNTIIDVVLSPSHGIELSVGEKL
VLNCTARTELNVGIDFNWEYPSSKHQHKKLVNRDLKTQSGSEMKKELSTLTIDGVTRSDQGLYTCAASSGLMT
KKNSTFVRVHEKDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFSCSV
MHEALHNHYTQKSLSLSPGK

In some embodiments, the nucleic acid sequence of aflibercept is codon-optimized for expression in a primate or a human subject. Construction of a synthetic gene corresponding to the aflibercept amino acid sequence has been described in literature, e.g., Kanda A, Noda K, Saito W, Ishida S. Aflibercept Traps Galectin-1, an Angiogenic Factor Associated with Diabetic Retinopathy. Scientific Reports 5:17946 (2015) (describing “VEGF-Trap_{R1R2} (corresponding to aflibercept) cDNA was generated as a synthetic gene by IDT (Coralville, IA)”). Given the available amino acid sequence of aflibercept, any method known in the art can be used to generate the cDNA of aflibercept for use in a gene therapy or a rAAV described herein.

Codon optimization can be achieved with any method known in the art. Codon optimization refers to a process of modifying a nucleic acid sequence for enhanced expression of a gene in target or host cells of interest, e.g., human retinal cells, by replacing at least one codon (e.g., about or more than 1, 2, 3, 4, 5, 10, 15, 20, 25, 50, 100 or more codons) of a native sequence with codons that are used more frequently or are most frequently used in the host cell while maintaining the native amino acid sequence. Codon usage tables are readily available, including for examples, GenScript Codon Usage Frequency Table Tool at www(dot)genscript(dot)com/tools/codon-frequency-table; Codon Usage Database at www(dot)kazusa(dot)or(dot)jp/codon/; and Nakamura, Y., et al. “Codon usage tabulated from the international DNA sequence databases: status for the year 2000” Nucl. Acids Res. 28:292 (2000).

Homology refers to the percent conservation of residues of an alignment between two sequences, including, but not limited to functional fragments, sequences comprising insertions, deletions, substitutions, pseudofragments, pseudogenes, splice variants or artificially optimized sequences.

In some embodiments, the rAAV particles comprise a nucleic acid encoding aflibercept. In some embodiments, the polypeptide is aflibercept.

As used herein, “aflibercept” refers to a polypeptide or protein sequence, or a functional fragment or variant or mutant thereof, with any of at least 75%, 80%, 81%, 82%, 83%, 84%, 85%, 86%, 87%, 88%, 89%, 90%, 91%, 92%, 93%, 94%, 95%, 96%, 97%, 98%, 99% or more, or 100% homology to the aflibercept amino acid sequence identified above (SEQ ID NO: 35). Homology refers to the percent conservation of residues of an alignment between two sequences, including, but not limited to functional fragments, sequences comprising insertions, deletions, substitutions, pseudofragments, pseudogenes, splice variants or artificially optimized sequences.

In some embodiments, the amino acid sequence of aflibercept is any of at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% homologous to the aflibercept amino acid sequence of SEQ ID NO: 35. In some embodiments, the nucleic acid sequence encoding aflibercept disclosed herein is compared to the corresponding cDNA sequence of the aflibercept amino acid sequence identified above, and shows any of at least 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% sequence homology between the nucleic acid sequences of aflibercept (e.g., SEQ ID NO: 36). In some embodiments, aflibercept is any of at least 80%, 85%, 90%, 91%, 92%, 93%, 94%, 95%, 96%, 97%, 98%, 99%, 99.9%, or 100% spatially homologous to aflibercept (e.g., in terms of its secondary, tertiary, and quaternary structure or conformation). In some embodiments, aflibercept is any of at most 80%, 85%, 90%, 91%, 92%, 93%, 94%, 95%, 96%, 97%, 98%, 99%, 99.9%, or 100% spatially homologous to the aflibercept used in the standard of care (e.g., secondary, tertiary, and quaternary structure or conformation).

In some embodiments, the aflibercept gene product, or aflibercept transgene, as included in a gene therapy based on a rAAV, comprises a capsid variant as disclosed herein (e.g., the 7m8 variant), encodes a protein, fusion protein, or polypeptide that has any of at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% homology to the above amino acid sequence of SEQ ID NO: 35, or between the corresponding cDNA sequences of aflibercept (e.g., cDNA of aflibercept sequence used in a gene therapy compared to SEQ ID NO: 36). In some embodiments, the methods compositions disclosed herein comprise a functional fragment of aflibercept, or a variant or mutant thereof. In some embodiments, the nucleic acid sequence of aflibercept is modified or codon-optimized to enhance its activity, expression, stability, and/or solubility in vivo.

Aflibercept is a 115 kDa fusion protein, which can be glycosylated. Aflibercept comprises an IgG backbone fused to extracellular VEGF receptor sequences of the human VEGFR-1 and VEGFR-2, and functions like a soluble decoy receptor by binding VEGF-A with a greater affinity than its natural or endogenous receptors. See, for example, Stewart M W. Aflibercept (VEGF Trap-eye): the newest anti-VEGF drug. Br. J. Ophthalmol. 2012 September; 96 (9): 1157-8. Aflibercept's high affinity for VEGF interferes or disrupts subsequent binding and activation of native or endogenous VEGF receptors. Reduced VEGF activity can lead to decreased angiogenesis and vascular permeability. Inhibition of placental growth factor PIGF and VEGF-B by aflibercept may also contribute to the treatment of ocular diseases or disorders characterized by abnormal (e.g., excessive) angiogenesis and/or neovascularization. PIGF has been associated with angiogenesis and certain ocular diseases or disorders, such as wet AMD, may be associated with elevated levels of PIGF. VEGF-B overexpression can be associated with breakdown of the blood-retinal barrier and retinal angiogenesis. Thus, inhibition of VEGF-A, VEGF-B, and PIGF may all contribute to the efficacy of aflibercept.

### Methods for Preparation of Vectors for Delivering Transgenes to Target Cells

In some embodiments, the rAAV particles are manufactured using any method known in the art. In some embodiments, the rAAV particles are manufactured using a baculovirus expression vector system in Sf9 cells. Sf9 cells are an insect cell culture cell line commonly used for recombinant protein production using baculovirus. In some embodiments, the rAAV particles are manufactured using two baculoviruses in Sf9 cells. In some embodiments, the rAAV particles are manufactured using two baculoviruses in Sf9 cells, wherein a first baculovirus encodes the genes for AAV2 Rep and AAV2.7m8 Cap proteins and a second baculovirus encodes an anti-VEGF agent. In some embodiments, the rAAV particles are manufactured using two baculoviruses in Sf9 cells, wherein a first baculovirus encodes the genes for AAV2 Rep and AAV2.7m8 Cap proteins and a second baculovirus encodes an aflibercept (e.g., human aflibercept) cDNA expression cassette. In some embodiments, the rAAV particles are manufactured using two baculoviruses in Sf9 cells, wherein a first baculovirus encodes the genes for AAV2 Rep and AAV2.7m8 Cap proteins and a second baculovirus comprises a nucleic acid encoding a polypeptide comprising an amino acid sequence with at least about 95% identity to the amino acid sequence of SEQ ID NO: 35 and flanked by AAV2 inverted terminal repeats (ITRs). In some embodiments, the polypeptide comprises the amino acid sequence of SEQ ID NO: 35. In some embodiments, the polypeptide is aflibercept.

### Doses

In some embodiments, the unit dose of rAAV particles is administered to one eye of the individual. In some embodiments, the one eye of the individual is the right eye or the left eye. In some embodiments, the one eye of the individual is the right eye. In some embodiments, the one eye of the individual is the left eye. In some embodiments, the methods provided herein further comprise administering a unit dose of rAAV particles to the contralateral eye of the individual. In some embodiments, the one eye of the individual is the right eye and the contralateral eye is the left eye. In some embodiments, the one eye of the individual is the left eye and the contralateral eye is the right eye.

In some embodiments, the administering the unit dose of rAAV particles to the contralateral eye is at least about 2 weeks (e.g., at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, at least about 3 years, at least about 4 years, at least about 5 years, or more) after administering the unit dose of rAAV particles to the one eye. In some embodiments, the administering the unit dose of rAAV particles to the contralateral eye is at least about 2 weeks after administering the unit dose of rAAV particles to the one eye and the unit dose of rAAV particles administered to the contralateral eye of the individual is higher (e.g., any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more, higher) than the unit dose of rAAV particles administered to the one eye of the individual.

In some embodiments, the administering the unit dose of rAAV particles to the contralateral eye of the individual is up to about 1 week, up to about 2 weeks, up to about 3 weeks, or up to about 4 weeks after administering the unit dose of rAAV particles to the one eye. In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye. In some embodiments, the administering the unit dose of rAAV particles to the contralateral eye of the individual is up to about 2 weeks (e.g., about 0 days, 1 day, 2 days, 3 days, 4 days, 5 days, 6 days, 7 days, 8 days, 9 days, 10 days, 11 days, 12 days, 13 days, or 14 days) after administering the unit dose of rAAV particles to the one eye and the unit dose of rAAV particles administered to the contralateral eye of the individual is about the same as (e.g., less than 1% higher or lower, less than 5% higher or lower, less than 10% higher or lower, or less than 20% higher or lower), or lower (e.g., about 5%, about 10%, about 20%, about 30%, about 40%, about 50%, about 60%, about 70%, about 80%, or about 90% lower) than the unit dose of rAAV particles administered to the one eye of the individual. In some embodiments, the administering the unit dose of rAAV particles to the contralateral eye of the individual is up to about 2 weeks after administering the unit dose of rAAV particles to the one eye and the unit dose of rAAV particles administered to the contralateral eye of the individual is about the same (e.g., less than 1% higher or lower, less than 5% higher or lower, less than 10% higher or lower, or less than 20% higher or lower) as the unit dose of rAAV particles administered to the one eye of the individual. In some embodiments, the administering the unit dose of rAAV particles to the contralateral eye of the individual is up to about 2 weeks after administering the unit dose of rAAV particles to the one eye and the unit dose of rAAV particles administered to the contralateral eye of the individual is lower (e.g., about 5%, about 10%, about 20%, about 30%, about 40%, about 50%, about 60%, about 70%, about 80%, or about 90% lower) than the unit dose of rAAV particles administered to the one eye of the individual.

In some embodiments, the unit dose of rAAV particles is administered to one eye and/or to the contralateral eye of the individual. In some embodiments, the unit dose of rAAV particles is expressed as the number of vector genomes (vg). In some embodiments, the unit dose is about 6×10¹¹ vector genomes (vg) or less of the rAAV particles. In some embodiments, the unit dose is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg of the rAAV particles, including any value within these ranges, of the rAAV particles. In some embodiments, the unit dose is about 6×10¹⁰ vector genomes (vg) to about 2×10¹¹ vg of the rAAV particles. In some embodiments, the unit dose is about 6×10¹⁰ vg to about 2×10¹¹ vg, about 7×10¹⁰ vg to about 2×10¹¹ vg, about 8×10¹⁰ vg to about 2×10¹¹ vg, about 9×10¹⁰ vg to about 2×10¹¹ vg, about 10×10¹⁰ vg to about 2×10¹¹ vg, or about 1×10¹¹ vg to about 2×10¹¹ vg of the rAAV particles. In some embodiments, the unit dose is about 6×10¹⁰ vg to about 2×10¹¹ vg of the rAAV particles. In some embodiments, the unit dose is about 6×10¹⁰ vg to about 7×10¹⁰ vg, about 7×10¹⁰ vg to about 8×10¹⁰ vg, about 8×10¹⁰ vg to about 9×10¹⁰ vg, about 9×10¹⁰ vg to about 10×10¹⁰ vg, about 10×10¹⁰ vg to about 1×10¹¹ vg, or about 1×10¹¹ vg to about 2×10¹¹ vg of the rAAV particles. In some embodiments, the unit dose is about 6×10¹⁰ vg, about 7×10¹⁰ vg, about 8×10¹⁰ vg, about 9×10¹⁰ vg, about 10×10¹⁰ vg, about 1×10¹¹ vg, or about 2×10¹¹ vg of the rAAV particles. In some embodiments, the unit dose is about 6×10¹⁰ vg or about 2×10¹¹ vg of the rAAV particles. In some embodiments, the unit dose is about 6×10¹⁰ vg of the rAAV particles. In some embodiments, the unit dose is about 6×10¹⁰ vg, about 2×10¹¹ vg, or about 6× 10¹¹ vg. In some embodiments, the unit dose is about 6×10¹⁰ vg. In some embodiments, the unit dose is about 2×10¹¹ vg. In some embodiments, the unit dose is about 6×10¹¹ vg.

In some embodiments, the unit dose of rAAV particles is administered to one eye and/or to the contralateral eye of the individual. In some embodiments, the unit dose is expressed as the number of vector genomes (vg) per eye (vg/eye). In some embodiments, the unit dose is about 6×10¹¹ vg/eye or less of the rAAV particles, e.g., 1×10¹¹ vg/eye. In some embodiments, the unit dose is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×10¹⁰ to about 6×10¹⁰, between about 6×101° to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg/eye of the rAAV particles, including any value within these ranges, of the rAAV particles. In some embodiments, the unit dose is about 6×10¹⁰ vg/eye to about 2×10¹¹ vg/eye of the rAAV particles. In some embodiments, the unit dose is about 6×10¹⁰ vg/eye to about 2×10¹¹ vg/eye, about 7×10¹⁰ vg/eye to about 2×10¹¹ vg/eye, about 8×10¹⁰ vg/eye to about 2×10¹¹ vg/eye, about 9×10¹⁰ vg/eye to about 2×10¹¹ vg/eye, about 10×10¹⁰ vg/eye to about 2×10¹¹ vg/eye, or about 1×10¹¹ vg/eye to about 2×10¹¹ vg/eye of the rAAV particles. In some embodiments, the unit dose is about 6×10¹⁰ vg/eye to about 2×10¹¹ vg/eye of the rAAV particles. In some embodiments, the unit dose is about 6×10¹⁰ vg/eye to about 7×10¹⁰ vg/eye, about 7×10¹⁰ vg/eye to about 8×10¹⁰ vg/eye, about 8×10¹⁰ vg/eye to about 9×10¹⁰ vg/eye, about 9×10¹⁰ vg/eye to about 10×10¹⁰ vg/eye, about 10×10¹⁰ vg/eye to about 1×10¹¹ vg/eye, or about 1×10¹¹ vg/eye to about 2×10¹¹ vg/eye of the rAAV particles. In some embodiments, the unit dose is about 6×10¹⁰ vg/eye, about 7×10¹⁰ vg/eye, about 8×10¹⁰ vg/eye, about 9×10¹⁰ vg/eye, about 10×10¹⁰ vg/eye, about 1×10¹¹ vg/eye, or about 2×10¹¹ vg/eye of the rAAV particles. In some embodiments, the unit dose is about 6×10¹⁰ vg/eye or about 2×10¹¹ vg/eye of the rAAV particles. In some embodiments, the unit dose is about 6×10¹⁰ vg/eye of the rAAV particles. In some embodiments, the unit dose is about 6×10¹⁰ vg/eye, about 2×10¹¹ vg/eye, or about 6×10¹¹ vg/eye. In some embodiments, the unit dose is about 6×10¹⁰ vg/eye. In some embodiments, the unit dose is about 2×10¹¹ vg/eye. In some embodiments, the unit dose is about 6×10¹¹ vg/eye.

In some embodiments, the unit dose of rAAV particles is administered to one eye and/or to the contralateral eye of the individual. In some embodiments, E is a shorthand for base 10 for exponentiation, and xEy refers to x multiplied by base 10 to the y power/exponent. In some embodiments, the unit dose is expressed as the number of vector genomes (vg). In some embodiments, the unit dose is about 6E¹¹ vector genomes (vg) or less of the rAAV particles. In some embodiments, the unit dose is about 1E¹⁰ to about 2E¹⁰ between about 2E¹⁰ to about 3E¹⁰, between about 3E¹⁰ to about 4E¹⁰, between about 4E¹⁰ to about 5E¹⁰, between about 5E¹⁰ to about 6E¹⁰, between about 6E¹⁰ to about 7E¹⁰, between about 7E¹⁰ to about 8E¹⁰, between about 8E¹⁰ to about 9E¹⁰, between about 9E¹⁰ to about 10E¹⁰, between about 1E¹¹ to about 2E¹¹, between about 2E¹¹ to about 3E¹¹, between about 3E¹¹ to about 4E¹¹, between about 4E¹¹ to about 5E¹¹, or between about 5E¹¹ to about 6E¹¹ vg of the rAAV particles, including any value within these ranges, of the rAAV particles. In some embodiments, the unit dose is about 6E¹⁰ vector genomes (vg) to about 2E¹¹ vg of the rAAV particles. In some embodiments, the unit dose is about 6E¹⁰ vg to about 2E¹¹ vg, about 7E¹⁰ vg to about 2E¹¹ vg, about 8E¹⁰ vg to about 2E¹¹ vg, about 9E¹⁰ vg to about 2E¹¹ vg, about 10E¹⁰ vg to about 2E¹¹ vg, or about 1E¹¹ vg to about 2E¹¹ vg of the rAAV particles. In some embodiments, the unit dose is about 6E¹⁰ vg to about 2E¹¹ vg of the rAAV particles. In some embodiments, the unit dose is about 6E¹⁰ vg to about 7E¹⁰ vg, about 7E¹⁰ vg to about 8E¹⁰ vg, about 8E¹⁰ vg to about 9E¹⁰ vg, about 9E¹⁰ vg to about 10E¹⁰ vg, about 10E¹⁰ vg to about 1E¹¹ vg, or about 1E¹¹ vg to about 2E¹¹ vg of the rAAV particles. In some embodiments, the unit dose is about 6E¹⁰ vg, about 7E¹⁰ vg, about 8E¹⁰ vg, about 9E¹⁰ vg, about 10E¹⁰ vg, about 1E¹¹ vg, or about 2E¹¹ vg of the rAAV particles. In some embodiments, the unit dose is about 6E¹⁰ vg or about 2E¹¹ vg of the rAAV particles. In some embodiments, the unit dose is about 6E¹⁰ vg of the rAAV particles. In some embodiments, the unit dose is about 6E¹⁰ vg, about 2E¹¹ vg, or about 6E¹¹ vg. In some embodiments, the unit dose is about 6E¹⁰ vg. In some embodiments, the unit dose is about 2E¹¹ vg. In some embodiments, the unit dose is about 6E¹¹ vg.

In some embodiments, the unit dose of rAAV particles is administered to one eye and/or to the contralateral eye of the individual. In some embodiments, the unit dose is expressed as the number of vector genomes (vg) per eye (vg/eye). In some embodiments, the unit dose is about 6E¹¹ vg/eye or less of the rAAV particles. In some embodiments, the unit dose is about 1E¹⁰ to about 2E¹⁰, between about 2E¹⁰ to about 3E¹⁰, between about 3E¹⁰ to about 4E¹⁰, between about 4E¹⁰ to about 5E¹⁰, between about 5E¹⁰ to about 6E¹⁰ between about 6E¹⁰ to about 7E¹⁰, between about 7E¹⁰ to about 8E¹⁰, between about 8E¹⁰ to about 9E¹⁰, between about 9E¹⁰ to about 10E¹⁰, between about 1E¹¹ to about 2E¹¹, between about 2E¹¹ to about 3E¹¹, between about 3E¹¹ to about 4E¹¹, between about 4E¹¹ to about 5E¹¹, or between about 5E¹¹ to about 6E¹¹ vg/eye of the rAAV particles, including any value within these ranges, of the rAAV particles. In some embodiments, the unit dose is about 6E¹⁰ vg/eye to about 2E¹¹ vg/eye of the rAAV particles. In some embodiments, the unit dose is about 6E¹⁰ vg/eye to about 2E¹¹ vg/eye, about 7E¹⁰ vg/eye to about 2E¹¹ vg/eye, about 8E¹⁰ vg/eye to about 2E¹¹ vg/eye, about 9E¹⁰ vg/eye to about 2E¹¹ vg/eye, about 10E¹⁰ vg/eye to about 2E¹¹ vg/eye, or about 1E¹¹ vg/eye to about 2E¹¹ vg/eye of the rAAV particles. In some embodiments, the unit dose is about 6E¹⁰ vg/eye to about 2E¹¹ vg/eye of the rAAV particles. In some embodiments, the unit dose is about 6E¹⁰ vg/eye to about 7E¹⁰ vg/eye, about 7E¹⁰ vg/eye to about 8E¹⁰ vg/eye, about 8E¹⁰ vg/eye to about 9E¹⁰ vg/eye, about 9E¹⁰ vg/eye to about 10E¹⁰ vg/eye, about 10E¹⁰ vg/eye to about 1E¹¹ vg/eye, or about 1E¹¹ vg/eye to about 2E¹¹ vg/eye of the rAAV particles. In some embodiments, the unit dose is about 6E¹⁰ vg/eye, about 7E¹⁰ vg/eye, about 8E¹⁰ vg/eye, about 9E¹⁰ vg/eye, about 10E¹⁰ vg/eye, about 1E¹¹ vg/eye, or about 2E¹¹ vg/eye of the rAAV particles. In some embodiments, the unit dose is about 6E¹⁰ vg/eye or about 2E¹¹ vg/eye of the rAAV particles. In some embodiments, the unit dose is about 6E¹⁰ vg/eye of the rAAV particles. In some embodiments, the unit dose is about 6E¹⁰ vg/eye, about 2E¹¹ vg/eye, or about 6E¹¹ vg/eye. In some embodiments, the unit dose is about 6E¹⁰ vg/eye. In some embodiments, the unit dose is about 2E¹¹ vg/eye. In some embodiments, the unit dose is about 6E¹¹ vg/eye. One skilled in the art recognizes that 1E # notation format is equivalent to the 1×10^{#} notation format.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the vitreous fluid. In some embodiments, the unit dose of rAAV particles is a unit dose sufficient to achieve a concentration of the therapeutic protein (e.g., an anti-VEGF agent such as aflibercept) at about any one of 3, 3.5, 4, 4.5, 5, 5.5, 6, 6.5, 7, 7.5, 8, 8.5, 9, 9.5, 10 μg/ml, or more, including any range in between these values, in the vitreous fluid. In some embodiments, the unit dose of rAAV particles is a unit dose sufficient to cause expression of aflibercept in the vitreous fluid. In some embodiments, the unit dose of rAAV particles is a unit dose sufficient to achieve a concentration of aflibercept at about any one of 3, 3.5, 4, 4.5, 5, 5.5, 6, 6.5, 7, 7.5, 8, 8.5, 9, 9.5, 10 μg/ml, or more, including any range in between these values, in the vitreous fluid.

In some embodiments, the unit dose of rAAV particles administered to the one eye and/or to the contralateral eye of the individual is a unit dose sufficient to cause expression of the therapeutic protein (e.g., an anti-VEGF agent such as aflibercept) in the aqueous fluid. In some embodiments, the unit dose of rAAV particles is a unit dose sufficient to achieve a concentration of the therapeutic protein (e.g., an anti-VEGF agent such as aflibercept) of at least about 0.5, 0.55, 0.6, 0.65, 0.7, 0.75, 0.8, 0.85, 0.9, 0.95, 1.0 μg/ml, or more, including any range in between these values, in the aqueous fluid. In some embodiments, the unit dose of rAAV particles is a unit dose sufficient to cause expression of aflibercept in the aqueous fluid. In some embodiments, the unit dose of rAAV particles is a unit dose sufficient to achieve a concentration of aflibercept of at least about 0.5, 0.55, 0.6, 0.65, 0.7, 0.75, 0.8, 0.85, 0.9, 0.95, 1.0 μg/ml, or more, including any range in between these values, in the aqueous fluid.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the retina. In some embodiments, the unit dose of rAAV particles is a unit dose sufficient to achieve a concentration of the therapeutic protein (e.g., an anti-VEGF agent such as aflibercept) of at least about 3, 3.5, 4, 4.5, 5, 5.5, 6, 6.5, 7, 7.5, 8, 8.5, 9, 9.5, 10 μg/g, or more, including any range in between these values, in the retina. In some embodiments, the unit dose of rAAV particles is a unit dose sufficient to cause expression of aflibercept in the retina. In some embodiments, the unit dose of rAAV particles is a unit dose sufficient to achieve a concentration of aflibercept of at least about 3, 3.5, 4, 4.5, 5, 5.5, 6, 6.5, 7, 7.5, 8, 8.5, 9, 9.5, 10 μg/g, or more, including any range in between these values, in the retina.

In some embodiments, the unit dose of rAAV particles is administered to one eye and/or to the contralateral eye of the individual. In some embodiments, the unit dose of rAAV particles is a unit dose sufficient to cause expression of the therapeutic protein (e.g., an anti-VEGF agent such as aflibercept) in the choroid. In some embodiments, the unit dose of rAAV particles is a unit dose sufficient to achieve a concentration of the therapeutic protein (e.g., an anti-VEGF agent such as aflibercept) at about any one of 3, 3.5, 4, 4.5, 5, 5.5, 6, 6.5, 7, 7.5, 8, 8.5, 9, 9.5, 10 μg/g, or more, including any range in between these values, in the choroid. In some embodiments, the unit dose of rAAV particles is a unit dose sufficient to cause expression of aflibercept in the choroid. In some embodiments, the unit dose of rAAV particles is a unit dose sufficient to achieve a concentration of aflibercept at about any one of 3, 3.5, 4, 4.5, 5, 5.5, 6, 6.5, 7, 7.5, 8, 8.5, 9, 9.5, 10 μg/g, or more, including any range in between these values, in the choroid.

In some embodiments, the unit dose of rAAV particles is administered to one eye and/or to the contralateral eye of the individual. In some embodiments, the unit dose of rAAV particles is a therapeutically effective dose.

In some embodiments, the unit dose of rAAV particles is a therapeutically effective dose if the unit dose is sufficient to cause maintenance or a decrease of retinal thickness compared to the retinal thickness prior to administration of the unit dose of rAAV particles. In some embodiments, the unit dose of rAAV particles is a therapeutically effective dose if the unit dose is sufficient to cause a decrease of retinal thickness compared to the retinal thickness prior to administration of the unit dose of rAAV particles. In some embodiments, retinal thickness is central subfield thickness (CST) or central retinal thickness (CRT). In some embodiments, the unit dose of rAAV particles is a therapeutically effective dose if the unit dose is sufficient to cause a decrease of retinal thickness of more than any of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% compared to the retinal thickness prior to administration of the unit dose of rAAV particles. In some embodiments, the retinal thickness (e.g., CST or CRT) is determined by OCT or SD-OCT.

In some embodiments, the unit dose of rAAV particles is a therapeutically effective dose if the unit dose is sufficient to cause maintenance or a decrease in macular volume compared to the macular volume prior to administration of the unit dose of rAAV particles. In some embodiments, the unit dose of rAAV particles is a therapeutically effective dose if the unit dose is sufficient to cause a decrease in macular volume compared to the macular volume prior to administration of the unit dose of rAAV particles. In some embodiments, the unit dose of rAAV particles is a therapeutically effective dose if the unit dose is sufficient to cause a decrease in macular volume of more than any of about 5%, about 10%, about 15%, about 20%, about 25%, about 30%, about 35%, about 40%, about 45%, about 50%, about 55%, about 60%, or about 65% compared to the macular volume prior to administration of the unit dose of rAAV particles. In some embodiments, the macular volume is determined by OCT or SD-OCT.

In some embodiments, the unit dose of rAAV particles is a therapeutically effective dose if the unit dose is sufficient to cause maintenance or an improvement of visual acuity compared to the visual acuity prior to administration of the unit dose of rAAV particles. In some embodiments, the unit dose of rAAV particles is a therapeutically effective dose if the unit dose is sufficient to cause an improvement of visual acuity compared to the visual acuity prior to administration of the unit dose of rAAV particles. In some embodiments, the unit dose of rAAV particles is a therapeutically effective dose if the unit dose is sufficient to cause an improvement of visual acuity of more than any of about 5%, about 10%, about 20%, about 30%, about 40%, about 50%, about 60%, about 70%, about 80%, about 90%, about 100%, about 125%, about 150%, about 175%, about 200%, about 225%, about 250%, about 275%, about 300%, or more compared to the visual acuity prior to administration of the unit dose of rAAV particles. In some embodiments, visual acuity is best corrected visual acuity (BCVA). In some embodiments, the unit dose of rAAV particles is a therapeutically effective dose if the unit dose is sufficient to cause an improvement of BCVA compared to the BCVA prior to administration of the unit dose of rAAV particles. In some embodiments, BCVA is expressed as an ETDRS score, which corresponds to the number of letters correctly read (Vitale et al., (2016) JAMA Opthalmol 134 (9): 1041:1047). In some embodiments, the unit dose of rAAV particles is a therapeutically effective dose if the unit dose is sufficient to cause an improvement of BCVA of at least 15 ETDRS letters (Vitale et al., (2016) JAMA Opthalmol 134 (9): 1041:1047) (e.g., at least about 15, at least about 20, at least about 30, at least about 40, at least about 50, at least about 60, or about 70 letters) compared to the BCVA prior to administration of the unit dose of rAAV particles. In some embodiments, the unit dose of rAAV particles is a therapeutically effective dose if the unit dose is sufficient to cause maintenance of BCVA, wherein the individual loses fewer than 15 ETDRS letters (Vitale et al., (2016) JAMA Opthalmol 134 (9): 1041:1047) (e.g., 15 or less, 14 or less, 13 or less, 12 or less, 11 or less, 10 or less, 9 or less, 8 or less, 7 or less, 6 or less, 5 or less, 4 or less, 3 or less, 2 or less, 1, or 0 letters) compared to the BCVA prior to administration of the unit dose of rAAV particles.

In some embodiments, the unit dose of rAAV particles is a therapeutically effective dose if, after administration of the unit dose of rAAV particles, the individual is determined to have maintenance of vision. In some embodiments, the unit dose of rAAV particles is a therapeutically effective dose if, after administration of the unit dose of rAAV particles, the individual is determined have an improvement of vision. In some embodiments, the unit dose of rAAV particles is a therapeutically effective dose if, after administration of the unit dose of rAAV particles, the CST or CRT assessed by SD-OCT is decreased compared to prior to administration of the unit dose of rAAV particles. In some embodiments, the unit dose of rAAV particles is a therapeutically effective dose if, after administration of the unit dose of rAAV particles, the CST or CRT assessed by SD-OCT is maintained compared to prior to administration of the unit dose of rAAV particles.

In some embodiments, the unit dose of rAAV particles is a therapeutically effective dose if, after administration of the unit dose of rAAV particles, the macular volume is decreased compared to prior to administration of the unit dose of rAAV particles. In some embodiments, the unit dose of rAAV particles is a therapeutically effective dose if, after administration of the unit dose of rAAV particles, the macular volume is maintained compared to prior to administration of the unit dose of rAAV particles.

In some embodiments, the unit dose of rAAV particles is a therapeutically effective dose if, after administration of the unit dose of rAAV particles, the retinal thickness (e.g., central retinal thickness (CRT) or central subfield thickness (CST)) and macular volume are decreased compared to prior to administration of the unit dose of rAAV particles. In some embodiments, the unit dose of rAAV particles is a therapeutically effective dose if, after administration of the unit dose of rAAV particles, the retinal thickness (e.g., central retinal thickness (CRT) or central subfield thickness (CST)) and macular volume are maintained compared to prior to administration of the unit dose of rAAV particles.

In some embodiments, the unit dose of rAAV particles is a therapeutically effective dose if, after administration of the unit dose of rAAV particles, the individual requires less than one rescue therapy treatment (e.g., aflibercept injection) about any of every 4 weeks, every 5 weeks, every 6 weeks, every 7 weeks, every 8 weeks, every 9 weeks, every 10 weeks, or more after administration of the unit dose of rAAV particles in the one eye and/or the contralateral eye. In some embodiments, the unit dose of rAAV particles is a therapeutically effective dose if, after administration of the unit dose of rAAV particles, the individual does not require any rescue therapy treatment (e.g., aflibercept injection) for at least about any of 1 week, at least 2 weeks, at least 3 weeks, at least 4 weeks, at least 5 weeks, at least 6 weeks, at least 7 weeks, at least 8 weeks, at least 9 weeks, at least 10 weeks, at least 15 weeks, at least 20 weeks, at least 30 weeks, at least 40 weeks, at least 50 weeks, at least 60 weeks, at least 70 weeks, at least 80 weeks, at least 90 weeks, at least 100 weeks, at least 110 weeks, or more.

In some embodiments, the unit dose of rAAV particles is a therapeutically effective dose if, after administration of the unit dose of rAAV particles, the individual is determined to have a reduction in retinal fluid compared to the level of retinal fluid prior to administration of the unit dose of rAAV particles in the one eye and/or the contralateral eye. In some embodiments, the unit dose of rAAV particles is a therapeutically effective dose if, after administration of the unit dose of rAAV particles, the individual is determined to have maintenance in retinal fluid compared to the level of retinal fluid prior to administration of the unit dose of rAAV particles in the one eye and/or the contralateral eye. In some embodiments, the unit dose of rAAV particles is a therapeutically effective dose if, after administration of the unit dose of rAAV particles, the individual is determined to have a reduction in IRF and/or SRF in the one eye and/or the contralateral eye compared to the levels of IRF and/or SRF prior to administration of the unit dose of rAAV particles in the one eye and/or the contralateral eye.

In some embodiments, the unit dose of rAAV particles is a therapeutically effective dose if, after administration of the unit dose of rAAV particles, the individual is determined to have a resolution of pigment epithelial detachment (PED) compared to PED prior to administration of the unit dose of rAAV particles in the one eye and/or the contralateral eye.

In some embodiments, the unit dose of rAAV particles is a therapeutically effective dose if, after administration of the unit dose of rAAV particles, CNV lesions shrink compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions shrink by more than any of about 5%, 10%, 20%, 30%, 40%, 50%, 60%, 70%, 80%, 90%, or 100% compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions do not grow compared to CNV lesions present prior to administration of the unit dose of rAAV particles in the one eye and/or the contralateral eye. In some embodiments, the unit dose of rAAV particles is a therapeutically effective dose if, after administration of the unit dose of rAAV particles, CNV lesions do not grow by more than about any of about 1%, 2%, 3%, 4%, 5%, 6%, 7%, 8%, 9%, 10%, 15%, or 20% compared to CNV lesions present prior to administration of the unit dose of rAAV particles in the one eye and/or the contralateral eye.

In some embodiments, the unit dose of rAAV particles is a therapeutically effective dose if, after administration of the unit dose of rAAV particles, the individual is determined to have an improvement in anatomical features of the one eye and/or the contralateral eye compared to the anatomical features prior to administration of the unit dose of rAAV particles. In some embodiments, the unit dose of rAAV particles is a therapeutically effective dose if, after administration of the unit dose of rAAV particles, the individual is determined to have a stabilization and/or maintenance of anatomical features of the one eye and/or the contralateral eye compared to the anatomical features prior to administration of the unit dose of rAAV particles.

In some embodiments, the unit dose of rAAV particles is therapeutically effective if administration of the dose to the one eye and/or the contralateral eye of the individual reduces, stops, or prevents at least one symptom of the ocular neovascular disease or disorder. In the cases of ocular neovascular diseases or disorders characterized by abnormal (e.g., excessive) angiogenesis, such symptoms include, but are not limited to, e.g., visual distortions (such as impaired color vision, blurred vision, deterioration of central vision) and vision loss. In some embodiments, the unit dose of rAAV particles administered to the one eye and/or to the contralateral eye of the individual is a therapeutically effective dose if administration of the unit dose to the one eye and/or to the contralateral eye of the individual results in the maintenance, partial resolution, or complete resolution of one or more clinical features of the ocular neovascular disease. For example, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular neovascular disease as measured by any method known in the art. In some embodiments, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular neovascular disease as assessed by best corrected visual acuity (BCVA) (e.g., based on an ETDRS score; Vitale et al., (2016) JAMA Opthalmol 134 (9): 1041:1047), central retinal thickness as determined by SD-OCT, the number of rescue therapy treatments (e.g., aflibercept injections) required by the individual after administration of the unit dose of rAAV particles in the one eye and/or the contralateral eye, the presence of intraretinal fluid (IRF) and/or subretinal fluid (SRF), the resolution of pigment epithelial detachment (PED), choroidal neovascularization (CNV) lesion growth, anatomical features based on any methods known in the art (e.g., SD-OCT, OCT, fluorescein angiography, digital color fundus photography, etc.). In some embodiments, the unit dose of rAAV particles administered to the one eye and/or to the contralateral eye of the individual is therapeutically effective if administration of the dose to the one eye and/or to the contralateral eye of the individual results in complete resolution, partial resolution or maintenance of the ocular neovascular disease as assessed by ophthalmologic examination, intraocular pressure (e.g., using a Goldmann applanation tonometer or Tono-pen), indirect ophthalmoscopy, examination of the one eye and/or the contralateral eye and adnexa, eyelid and/or pupil responsiveness, belpharoptosis, abnormal pupil shape, unequal pupils, abnormal reaction to light, afferent pupillary defects, slit-lamp examination (including of the eyelids, conjunctiva, cornea, lens, iris, and anterior chamber), posterior segment abnormalities of the vitreous, optic nerve, peripheral retina, and retinal vasculature, SD-OCT, fluorescein angiography, digital color fundus photography (including images of the retina, optic disc, and/or macula), aqueous humor sampling, vitreous humor sampling, OCT-angiography (OCT-A), refraction, and visual acuity (BCVA).

In some embodiments, the unit dose of rAAV particles administered to the one eye of the individual is the same as the unit dose of rAAV particles administered to the contralateral eye of the individual. In some embodiments, the unit dose of rAAV particles administered to the one eye of the individual is different from the unit dose of rAAV particles administered to the contralateral eye of the individual. In some embodiments, the unit dose of rAAV particles administered to the one eye of the individual is higher, e.g., more than any of about 10%, about 20%, about 30%, about 40%, about 50%, about 60%, about 70%, about 80%, about 90%, about 100%, about 125%, about 150%, about 175%, about 200%, about 225%, about 250%, about 275%, about 300% or more, than the unit dose of rAAV particles administered to the contralateral eye of the individual. In some embodiments, the unit dose of rAAV particles administered to the contralateral eye of the individual is higher, e.g., more than any of about 10%, about 20%, about 30%, about 40%, about 50%, about 60%, about 70%, about 80%, about 90%, about 100%, about 125%, about 150%, about 175%, about 200%, about 225%, about 250%, about 275%, about 300% or more, than the unit dose of rAAV particles administered to the one eye of the individual. In some embodiments, the unit dose of rAAV particles is expressed as the number of vector genomes (vg) per eye (vg/eye). In some embodiments, the unit dose of rAAV particles is about 6×10¹¹ vg/eye or less of the rAAV particles. In some embodiments, the unit dose of rAAV particles is about 1×10¹⁰ to about 2×10¹⁰, between about 2×10¹⁰ to about 3×10¹⁰, between about 3×10¹⁰ to about 4×10¹⁰, between about 4×10¹⁰ to about 5×10¹⁰, between about 5×101° to about 6×10¹⁰, between about 6×10¹⁰ to about 7×10¹⁰, between about 7×10¹⁰ to about 8×10¹⁰, between about 8×10¹⁰ to about 9×10¹⁰, between about 9×10¹⁰ to about 10×10¹⁰, between about 1×10¹¹ to about 2×10¹¹, between about 2×10¹¹ to about 3×10¹¹, between about 3×10¹¹ to about 4×10¹¹, between about 4×10¹¹ to about 5×10¹¹, or between about 5×10¹¹ to about 6×10¹¹ vg/eye of the rAAV particles, including any value within these ranges, of the rAAV particles. In some embodiments, the unit dose of rAAV particles is about 6×10¹⁰ vg/eye to about 2×10¹¹ vg/eye of the rAAV particles. In some embodiments, the unit dose of rAAV particles is about 6×10¹⁰ vg/eye to about 2×10¹¹ vg/eye, about 7×10¹⁰ vg/eye to about 2×10¹¹ vg/eye, about 8×10¹⁰ vg/eye to about 2×10¹¹ vg/eye, about 9×10¹⁰ vg/eye to about 2×10¹¹ vg/eye, about 10×10¹⁰ vg/eye to about 2×10¹¹ vg/eye, or about 1×10¹¹ vg/eye to about 2×10¹¹ vg/eye of the rAAV particles. In some embodiments, the unit dose of rAAV particles is about 6×10¹⁰ vg/eye to about 2×10¹¹ vg/eye of the rAAV particles. In some embodiments, the unit dose of rAAV particles is about 6×10¹⁰ vg/eye to about 7×10¹⁰ vg/eye, about 7×10¹⁰ vg/eye to about 8×10¹⁰ vg/eye, about 8×10¹⁰ vg/eye to about 9×10¹⁰ vg/eye, about 9×10¹⁰ vg/eye to about 10×10¹⁰ vg/eye, about 10×10¹⁰ vg/eye to about 1×10¹¹ vg/eye, or about 1×10¹¹ vg/eye to about 2×10¹¹ vg/eye of the rAAV particles. In some embodiments, the unit dose of rAAV particles is about 6×10¹⁰ vg/eye, about 7×10¹⁰ vg/eye, about 8×10¹⁰ vg/eye, about 9×10¹⁰ vg/eye, about 10×10¹⁰ vg/eye, about 1×10¹¹ vg/eye, or about 2×10¹¹ vg/eye of the rAAV particles. In some embodiments, the unit dose of rAAV particles is about 6×10¹⁰ vg/eye or about 2×10¹¹ vg/eye of the rAAV particles. In some embodiments, the unit dose of rAAV particles is about 6×10¹⁰ vg/eye of the rAAV particles. In some embodiments, the unit dose of rAAV particles is about 6×10¹⁰ vg/eye, about 2×10¹¹ vg/eye, or about 6× 10¹¹ vg/eye. In some embodiments, the unit dose of rAAV particles is about 6×10¹⁰ vg/eye. In some embodiments, the unit dose of rAAV particles is about 2×10¹¹ vg/eye. In some embodiments, the unit dose of rAAV particles is about 6×10¹¹ vg/eye.

In some embodiments, the unit dose of rAAV particles administered to the one eye of the individual and the unit dose of rAAV particles administered to the contralateral eye of the individual are administered at the same time. In some embodiments, the unit dose of rAAV particles administered to the one eye of the individual and the unit dose of rAAV particles administered to the contralateral eye of the individual are administered at different times. In some embodiments, the unit dose administered to the contralateral eye is administered any of at least about 1 hour, at least about 2 hours, at least about 4 hours, at least about 8 hours, at least about 12 hours, at least about 24 hours, at least about 1 day, at least about 2 days, at least about 3 days, at least about 4 days, at least about 5 days, at least about 6 days, at least about 7 days, at least about 1 week, at least about 2 weeks, at least about 3 weeks, at least about 4 weeks, or more after administering of the unit dose to the one eye. In some embodiments, the unit dose administered to the contralateral eye is administered at least about 2 weeks after administering of the unit dose to the one eye.

In some embodiments, a single unit dose of rAAV particles is administered to the one eye and/or the contralateral eye of the individual. In some embodiments, the single unit dose of rAAV particles administered to the one eye and/or to the contralateral eye is a therapeutically effective dose. In some embodiments, more than one dose of rAAV particles (e.g., more than any of about 2, 3, 4, 5, or more unit doses) are administered to the one eye and/or the contralateral eye of the individual. In some embodiments, the more than one doses of rAAV particles administered to the one eye and/or to the contralateral are therapeutically effective doses.

In some embodiments, an anti-VEGF treatment, e.g., an IVT injection with an anti-VEGF agent such as aflibercept, is administered to the one eye and/or to the contralateral eye administered the rAAV particles at least about one week, e.g. at least about 7 days, prior to administration of the unit dose of rAAV particles (e.g., a unit dose of between about 2×10¹¹ vg/eye to about 6×10¹¹ vg/eye of rAAV particles). In some embodiments, an anti-VEGF treatment, e.g., an IVT injection with an anti-VEGF agent such as aflibercept, is administered to an eye on about Day 1 and the unit dose of rAAV particles, e.g., a unit dose of between about 2×10¹¹ vg/eye to about 6×10¹¹ vg/eye of rAAV particles, is administered to the eye on about Day 8. In some embodiments, the unit dose of rAAV particles is about 2×10¹¹ vg/eye or about 6×10¹¹ vg/eye of rAAV particles. In some embodiments, the ocular neovascular disease is diabetic macular edema.

### Pharmaceutical Formulations

In some embodiments, the unit dose of rAAV particles is in a pharmaceutical formulation. In some embodiments, the pharmaceutical formulation comprises the rAAV particles, one or more osmotic or ionic strength agents, one or more buffering agents, one or more surfactants, and one or more solvents. In some embodiments, the osmotic or ionic strength agent is sodium chloride. In some embodiments, the one or more buffering agents are sodium phosphate monobasic and/or sodium phosphate dibasic. In some embodiments, the surfactant is Poloxamer 188. In some embodiments, the solvent is water. In some embodiments, the pharmaceutical formulation comprises the rAAV particles, sodium chloride, sodium phosphate monobasic, sodium phosphate dibasic, and a surfactant.

In some embodiments, the pharmaceutical formulation comprises about 1×10¹⁰ vg/mL to about 1×10¹³ vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹¹ vg/mL to about 6×10¹² vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹¹ vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5. In some embodiments, the pharmaceutical formulation comprises about 6×10¹² vg/mL of rAAV particles, about 150 mM to about 200 mM sodium chloride, about 1 mM to about 10 mM monobasic sodium phosphate, about 1 mM to about 10 mM dibasic sodium phosphate, and about 0.0005% (w/v) to about 0.005% (w/v) poloxamer 188, wherein the pharmaceutical formulation has a pH of about 7.0 to about 7.5.

In some embodiments, the rAAV particles in the pharmaceutical formulation are present at a concentration of about 1×10¹⁰ vg/ml to about 1×10¹³ vg/ml. In some embodiments, the rAAV particles in the pharmaceutical formulation are present at a concentration of about 1×10⁰⁹ vg/ml to about 6×10¹⁴ vg/ml. In certain embodiments, the rAAV particles in the pharmaceutical formulation are present at a concentration of about 1×10⁰⁹ vg/ml to about 2×10⁰⁹ vg/ml, about 2×10⁰⁹ vg/ml to about 3×10⁰⁹, about 3×10⁰⁹ vg/ml to about 4×10⁰⁹, about 4×10⁰⁹ vg/ml to about 5×10⁰⁹, about 5×10⁰⁹ vg/ml to about 6×10⁰⁹, about 6×10⁰⁹ vg/ml to about 7×10⁰⁹, about 7×10⁰⁹ vg/ml to about 8×10⁰⁹, about 8×10⁰⁹ vg/ml to about 9×10⁰⁹, about 9×10⁰⁹ vg/ml to about 10×10⁰⁹, about 10×10⁰⁹ vg/ml to about 1×10¹⁰ about 1×10¹⁰ vg/ml to about 2×10¹⁰, about 2×10¹⁰ vg/ml to about 3×10¹⁰, about 3×10¹⁰ vg/ml to about 4×10¹⁰, about 4×10¹⁰ vg/ml to about 5×10¹⁰, about 5×10¹⁰ vg/ml to about 6×10¹⁰, about 6×10¹⁰ vg/ml to about 7×10¹⁰, about 7×10¹⁰ vg/ml to about 8×10¹⁰, about 8×10¹⁰ vg/ml to about 9×10¹⁰, about 9×10¹⁰ vg/ml to about 10×10¹⁰, about 10×10¹⁰ vg/ml to about 1×10¹¹, about 1×10¹¹ vg/ml to about 2×10¹¹, about 2×10¹¹ vg/ml to about 3×10¹¹, about 3×10¹¹ vg/ml to about 4×10¹¹, about 4×10¹¹ vg/ml to about 5×10¹¹, about 5×10¹¹ vg/ml to about 6×10¹¹ about 6×10¹¹ vg/ml to about 7×10¹¹, about 7×10¹¹ vg/ml to about 8×10¹¹, about 8×10¹¹ vg/ml to about 9×10¹¹, about 9×10¹¹ vg/ml to about 10×10¹¹, about 1×10¹² vg/ml to about 2×10¹², about 2×10¹² vg/ml to about 3×10¹², about 3×10¹² vg/ml to about 4×10¹², about 4×10¹² vg/ml to about 5×10¹², about 5×10¹² vg/ml to about 6×10¹², about 6×10¹² vg/ml to about 7×10¹², about 7×10¹² vg/ml to about 8×10¹², about 8×10¹² vg/ml to about 9×10¹², about 9×10¹² vg/ml to about 10×10¹², about 1×10¹³ vg/ml to about 2×10¹³, about 2×10¹³ vg/ml to about 3×10¹³, about 3×10¹³ vg/ml to about 4×10¹³, about 4×10¹³ vg/ml to about 5×10¹³, about 5×10¹³ vg/ml to about 6×10¹³, about 6×10¹³ vg/ml to about 7×10¹³, about 7×10¹³ vg/ml to about 8×10¹³ about 8×10¹³ vg/ml to about 9×10¹³, about 9×10¹³ vg/ml to about 10×10¹³, about 1×10¹⁴ vg/ml to about 2×10¹⁴, about 2×10¹⁴ vg/ml to about 3×10¹⁴, about 3×10¹⁴ vg/ml to about 4×10¹⁴, about 4×10¹⁴ vg/ml to about 5×10¹⁴, or about 5×10¹⁴ vg/ml to about 6×10¹⁴ vg/mL. In some embodiments, the pharmaceutical formulation comprises about 6×10¹¹ vg/mL to about 6×10¹² vg/mL of rAAV particles. In some embodiments, the pharmaceutical formulation comprises about 6×10¹² vg/mL of rAAV particles. In some embodiments, the pharmaceutical formulation comprises about 6×10¹¹ vg/mL of rAAV particles.

In some embodiments, the sodium chloride in the pharmaceutical formulation is present at a concentration of about 150 mM to about 200 mM. In certain embodiments, the sodium chloride in the pharmaceutical formulation is present at a concentration of about 150 mM, about 160 mM, about 170 mM, about 180 mM, about 190 mM, or about 200 mM. In certain embodiments, the sodium chloride in the pharmaceutical formulation is present at a concentration of about 180 mM.

In some embodiments, the sodium phosphate monobasic is present in the pharmaceutical formulation at a concentration of about 1 mM to about 10 mM. In some embodiments, the sodium phosphate monobasic is present in the pharmaceutical formulation at a concentration of any of about 1 mM, about 2 mM, about 3 mM, about 4 mM, about 5 mM, about 6 mM, about 7 mM, about 8 mM, about 9 mM, or about 10 mM. In certain embodiments, the sodium phosphate monobasic is present in the pharmaceutical formulation at a concentration of about 5 mM.

In some embodiments, the sodium phosphate dibasic is present in the pharmaceutical formulation at a concentration of about 1 mM to about 10 mM. In some embodiments, the sodium phosphate dibasic is present in the pharmaceutical formulation at a concentration of any of about 1 mM, about 2 mM, about 3 mM, about 4 mM, about 5 mM, about 6 mM, about 7 mM, about 8 mM, about 9 mM, or about 10 mM. In certain embodiments, the sodium phosphate dibasic is present in the pharmaceutical formulation at a concentration of about 5 mM.

In some embodiments, the Poloxamer 188 is present in the pharmaceutical formulation at a concentration of about 0.0005% (w/v) to about 0.005% (w/v). In some embodiments, the Poloxamer 188 is present in the pharmaceutical formulation at a concentration of any of about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), or about 0.005% (w/v). In certain embodiments, the Poloxamer 188 is present in the pharmaceutical formulation at a concentration of about 0.001% (w/v).

In some embodiments, the pharmaceutical formulation has a pH of about 7.0 to about 7.5. In some embodiments, the pharmaceutical formulation has a pH of about 7.0, about 7.1, about 7.2, about 7.3, about 7.4, or about 7.5. In certain embodiments, the pharmaceutical formulation has a pH of about 7.3. In some embodiments, hydrochloric acid and sodium hydroxide are used to adjust the pH of the pharmaceutical formulation.

In some embodiments, the pharmaceutical formulation comprises about 6×10¹² vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3. In some embodiments, the pharmaceutical formulation comprises about 6×10¹¹ vg/mL of rAAV particles, about 180 mM sodium chloride, about 5 mM monobasic sodium phosphate, about 5 mM dibasic sodium phosphate, and about 0.001% (w/v) poloxamer 188, wherein the pharmaceutical formulation has a pH of about 7.3.

In some embodiments, the pharmaceutical formulations are suitable for administration to the one eye and/or the contralateral eye of the individual, e.g., a human patient, via intravitreal (IVT) injection to achieve a desired therapeutic or prophylactic effect. In some embodiments, the pharmaceutical formulation is supplied as a reconstituted homogenous solution. In some embodiments, the solution is a suspension. In some embodiments, the pharmaceutical formulation is supplied as a frozen suspension, and is thawed prior to administration to the one eye and/or the contralateral eye of the individual. In some embodiments, the solution is isotonic.

In other embodiments, the pharmaceutical composition comprising e.g., an AAV2.7m8 vector that comprises a nucleic acid sequence encoding the anti-VEGF agent (e.g., aflibercept or a functional fragment or variant thereof), is supplied in a lyophilized form, and is reconstituted prior to administration to the one eye and/or the contralateral eye of the individual. In some embodiments, the methods provided herein further comprise the steps of reconstituting, dissolving, or solubilizing a lyophilized pharmaceutical composition comprising rAAV (e.g., AAV2.7m8) and encoding the anti-VEGF agent (e.g., aflibercept or a functional fragment or variant thereof) in a buffer prior to administration to the subject. In some embodiments, such lyophilized pharmaceutical composition comprises one or more of the following: a cryoprotectant, a surfactant, a salt, a stabilizer, or any combination thereof.

In some embodiments, the pharmaceutical formulation is a homogenous solution. In some embodiments, the homogenous solution is supplied in a pre-filled syringe. In some embodiments, the pharmaceutical formulation is supplied as a suspension. In some embodiments, a suspension is a solution. In some embodiments, the suspension is refrigerated. In some embodiments, the suspension is frozen. In some embodiments, methods provided herein further comprise the step of warming the refrigerated suspension to room temperature and/or agitating the suspension to ensure that the active ingredient(s) are dissolved and/or evenly distributed in solution prior to administering to the one eye and/or the contralateral eye of the individual (e.g., via IVT injection). In some embodiments, methods provided herein further comprise the step of thawing the frozen suspension and warming to room temperature and/or agitating the suspension to ensure that the active ingredient(s) are dissolved and/or evenly distributed in solution prior to administering to the one eye and/or the contralateral eye of the individual (e.g., via IVT injection). In some embodiments, the suspension is diluted prior to administration to the subject (e.g., via IVT injection). In some embodiments, the suspension is supplied as a pre-filled syringe.

In some embodiments, the pharmaceutical formulation is provided as a frozen suspension. In some embodiments, the suspension comprises a pharmaceutically acceptable excipient, e.g., surfactant, glycerol, non-ionic surfactant, buffer, glycol, salt, and any combination thereof.

In some embodiments, the suspension is a solution. In some embodiments, the suspension comprises micelles.

In some embodiments, for storage stability and convenience of handling, a pharmaceutical formulation, comprising rAAV (e.g., AAV2.7m8) and a nucleic acid sequence that encodes the anti-VEGF agent (e.g., aflibercept or a functional fragment or variant thereof), is formulated as a lyophilized, freeze dried, or vacuum dried powder that is reconstituted with saline, buffer, or water prior to administration to the one eye and/or the contralateral eye of the individual. Alternately, the pharmaceutical formulation is formulated as an aqueous solution, such as a suspension or a homogeneous solution. A pharmaceutical formulation can contain rAAV particles comprising a nucleic acid sequence that encodes aflibercept. Various excipients, such as phosphate, PBS, or Tris buffer, glycol, glycerol, saline, surfactant (e.g., pluronic or polysorbate), or any combination thereof, can be used to stabilize a pharmaceutical formulation. Additionally, cryoprotectants, such as alcohols can be used as a stabilizer under freezing or drying conditions. In some embodiments, the gene therapy is provided as a suspension, a refrigerated suspension, or a frozen suspension.

In some embodiments, a suspension of the pharmaceutical formulation as disclosed herein has a volume of any of about 20 μL, 30 μL, 40 μL, 50 μL, 60 μL, 70 μL, 80 μL, 90 μL, 100 μL, 200 μL, 300 μL, 400 μL, 500 μL, 600 μL, 700 μL, 800 μL, 900 μL, or 1000 μL. In some embodiments, a suspension of the pharmaceutical formulation as disclosed herein has a volume of about 250 μL. In some embodiments, the suspension of the pharmaceutical formulation as disclosed herein has a volume of between 0.1 to 0.5 mL, between 0.1 to 0.2 mL, between 0.3 to 0.5 mL, between 0.5-1.0 mL, between 0.5-0.7 mL, between 0.6 to 0.8 mL, between 0.8 to 1 mL, between 0.9 to 1.1 mL, between 1.0 to 1.2 mL, or between 1.0 to 1.5 mL. In other embodiments, the volume is no more than 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL, or 1.5 mL. In some embodiments, the suspension of the pharmaceutical formulation as disclosed herein has a volume of about 0.25 mL.

In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal). In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with, a ready-to-use stopper (e.g., a stopper made of chlorobutyl), and sealed (e.g., with a sterile aluminum tear-off seal), wherein the vial contains a volume of between 0.1 to 0.5 mL, between 0.1 to 0.2 mL, between 0.2 to 0.3 mL, between 0.3 to 0.4 mL, or between 0.4 mL to 0.5 mL of the suspension of the pharmaceutical formulation. In some embodiments, a suspension of the pharmaceutical formulation as disclosed herein is provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of about 0.25 mL of the suspension of the pharmaceutical formulation.

In some embodiments, pharmaceutical formulations disclosed herein are designed, engineered, or adapted for administration to a primate (e.g., non-human primate and human subjects) via intravitreal or subretinal injection. In some embodiments, a pharmaceutical formulation comprising rAAV particles comprising a nucleic acid sequence that encodes the anti-VEGF agent (e.g., aflibercept) is formulated for intravitreal injection into an eye of an individual. In some embodiments, the pharmaceutical composition is formulated to or reconstituted to a concentration that allows intravitreal injection of a volume not more than about or not more than any of 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL. In some embodiments, a unit dose of the pharmaceutical formulation comprises a volume not more than about or not more than any of 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL. In some embodiments, methods disclosed herein comprise intravitreal injection of a volume of any of about 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 30 μL or about 100 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 30 μL of a solution or suspension of a pharmaceutical formulation comprising a rAAV (e.g., AAV2.7m8) and a nucleic acid sequence that encodes the anti-VEGF agent (e.g., aflibercept). In some embodiments, methods disclosed herein comprise intravitreal injection of a volume of about 100 μL of a solution or suspension of a pharmaceutical formulation comprising a rAAV (e.g., AAV2.7m8) and a nucleic acid sequence that encodes the anti-VEGF agent (e.g., aflibercept).

In some embodiments, an AAV2.7m8 particle comprising a nucleic acid sequence of the anti-VEGF agent (e.g., aflibercept) transgene described herein is a component of a gene therapy pharmaceutical formulation. In some embodiments, a rAAV particle of any serotype comprising the 7m8 variant capsid protein as described herein is used to make a frozen suspension or a freeze-dried or lyophilized formulation composition. In some embodiments, the gene therapy is formulated as a refrigerated or frozen suspension. In some embodiments, the rAAV particle is rAAV2. In some embodiments, the lyophilized or suspension of the pharmaceutical formulation comprises rAAV2 comprising the 7m8 variant capsid protein and a DNA sequence that encodes the anti-VEGF agent (e.g., aflibercept). In some embodiments, the suspension is refrigerated or frozen.

In some embodiments, the administration of the unit dose of rAAV particles to the one eye and/or to the contralateral eye of the individual is by intravitreal (IVT) injection. For IVT injection, the rAAV particles can be delivered in the form of a suspension of a pharmaceutical formulation (e.g., as described herein). Initially, topical anesthetic is applied to the surface of the eye followed by an ophthalmic antiseptic solution. The eye is held open, with or without instrumentation, and the rAAV particles are injected through the sclera with a short, narrow needle, e.g., a 30-gauge needle, into the vitreous cavity of the one eye and/or the contralateral eye of the individual under direct observation. Typically, a volume of between about 25 μL to about 250 μL (e.g., any of about 25 μL, about 30 μL, about 40 μL about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, or about 250 μL) of an rAAV particle suspension may be delivered to the eye by IVT injection. In some embodiments, the unit dose of rAAV particles comprises a volume of about 100 μL. In some embodiments, the unit dose of rAAV particles comprises a volume of about 30 μL. In some embodiments, the IVT injection is performed in combination with removal of vitreous fluid. In some embodiments, a vitrectomy may be performed, and the entire volume of vitreous gel is replaced by an infusion of the rAAV particle suspension (e.g., about 4 mL of the rAAV particle suspension). A vitrectomy is performed using a cannula of appropriate bore size (e.g., 20 gauge to 27 gauge), wherein the volume of vitreous gel that is removed is replaced by infusion of fluid, e.g., saline, an isotonic solution, a rAAV particle suspension, from the infusion cannula. IVT administration is generally well tolerated. At the conclusion of the procedure, there is sometimes mild redness at the injection site. There is occasional tenderness, but most patients do not report any pain. No eye patch or eye shield is necessary after this procedure, and activities are not restricted. Sometimes, an antibiotic eye drop is prescribed for several days to help prevent infection.

In some embodiments, the pharmaceutical formulation is a unit dose (e.g., a therapeutically effective dose) to be administered to the one eye and/or the contralateral eye of an individual (e.g., a human or non-human primate) via IVT injection for the treatment of an ocular disease or disorder characterized by abnormal (e.g., excessive) angiogenesis or neovascularization. In some embodiments, the pharmaceutical formulation comprises a unit dose (e.g., a therapeutically effective dose) as described in further detail elsewhere herein. In some embodiments, the volume of the unit dose (e.g., a therapeutically effective dose) of a viral vector (e.g., an rAAV vector disclosed herein) administered to the subject is no more than any one of about 25 μL, 30 μL, 35 μL, 40 μL, 45 μL, 50 μL, 55 μL, 60 μL, 65 μL, 70 μL, 75 μL, 80 μL, 85 μL, 90 μL, 95 μL, 100 μL, 110 μL, 120 μL, 130 μL, 140 μL, 150 μL, 160 μL, 170 μL, 180 μL, 190 μL, 200 μL, 210 μL, 220 μL, 230 μL, 240 μL, or 250 μL, including any range in between these values. Minimizing the volume of the unit dose to be administered to the subject may obviate or mitigate changes in ocular pressure and other adverse effects associated with IVT injection (e.g., elevated intraocular pressure, inflammation, irritation, or pain).

Pharmaceutical formulations suitable for ocular use include sterile aqueous solutions or dispersions and sterile powders for the extemporaneous preparation of sterile injectable solutions, suspension, or dispersion. For intravitreal administration, suitable carriers include physiological saline, bacteriostatic water, phosphate buffered saline (PBS), and/or an isotonic agent, e.g., glycerol. In certain embodiments, the pharmaceutical formulation is sterile and fluid to the extent that easy syringability or injectability exists. In certain embodiments, the pharmaceutical formulation is stable under the conditions of manufacture and storage and is preserved against the contaminating action of microorganisms such as bacteria and fungi. In some embodiments, the pharmaceutical composition can include an isotonic agent, such as a salt or glycerol. In some embodiments, a surfactant or a stabilizer is added to the pharmaceutical composition to prevent aggregation.

In some embodiments, the pharmaceutical formulation contains an excipient or a carrier. A carrier is a solvent or dispersion medium containing, for example, water, saline, ethanol, a polyol (for example, glycerol, propylene glycol, and liquid polyethylene glycol, and the like), and any combination thereof. The proper fluidity can be maintained, for example, by the use of a coating such as lecithin, by the maintenance of the required particle size in the case of dispersion and by the use of surfactants such as polysorbates (e.g., Tween™, polysorbate 20, polysorbate 80), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™), cyclodextrins, and ethylbenzethonium chloride (Hyamine™). Prevention of the action of microorganisms can be achieved by various antibacterial and antifungal agents, for example, parabens, chlorobutanol, phenol, ascorbic acid, cresol, thimerosal, and the like. In many embodiments, isotonic agents are included in the pharmaceutical formulation, for example, sugars, polyalcohols such as mannitol, sorbitol, and/or sodium chloride. Prolonged absorption of the internal compositions can be brought about by including in the composition an agent that delays absorption, for example, aluminum monostearate and gelatin. In some embodiments, the pharmaceutical carrier includes sodium phosphate, sodium chloride, polysorbate, and sucrose. In some embodiments, a pharmaceutical formulation comprises a surfactant, e.g., non-ionic surfactant such as polysorbate, poloxamer, or pluronic. In some embodiments, the addition of a non-ionic surfactant reduces aggregation in the pharmaceutical composition.

Also provided herein are kits comprising at least one pharmaceutical formulation described herein. In some embodiments, the kit comprises a frozen suspension of a pharmaceutical formulation (e.g., one unit dose in a vial). In some embodiments, the kit comprises a lyophilized or freeze-dried pharmaceutical formulation (e.g., one unit dose in a vial) disclosed herein and a solution for dissolving, diluting, and/or reconstituting the lyophilized pharmaceutical composition. In some embodiments, the solution for reconstituting or dilution is supplied as a pre-filled syringe. In some embodiments, a kit comprises a freeze-dried or lyophilized pharmaceutical composition comprising rAAV (e.g., AAV2.7m8) and a solution for reconstituting the pharmaceutical composition to a desired concentration or volume. In some embodiments, the kit includes a buffer that helps to prevent aggregation upon reconstituting the pharmaceutical composition disclosed herein. In some embodiments, the pharmaceutical composition is provided in a pre-filled syringe. In some embodiments, a kit comprises a dual-chamber syringe or container wherein one of the chambers contains a buffer for dissolving or diluting the pharmaceutical composition. In some embodiments, the kit comprises a syringe for injection. In some embodiments, the reconstituted solution is filtered before administration. In some embodiments, the kit comprises a filter or a filter syringe for filtering the reconstituted pharmaceutical composition before administration to a patient. In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal). In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of between 0.1 to 0.5 mL, between 0.1 to 0.2 mL, between 0.2 to 0.3 mL, between 0.3 to 0.4 mL, or between 0.4 mL to 0.5 mL of the suspension of the pharmaceutical formulation. In some embodiments, the kit comprises a suspension of the pharmaceutical formulation comprising the rAAV particles as disclosed herein provided as a sterile-filtered, frozen suspension in a sterile, ready-to-use vial (e.g., a 0.5 mL vial; e.g., a Crystal Zenith® vial) with a ready-to-use stopper (e.g., a stopper made of chlorobutyl), and sealed (e.g., with a sterile aluminum tear-off seal), wherein the vial contains a volume of about 0.25 mL of the suspension of the pharmaceutical formulation. In some embodiments, the kit further comprises instructions for use, e.g., instructions for treating an ocular neovascular disease with the rAAV particles disclosed herein.

### Ocular Neovascular Diseases

In one aspect, the present disclosure provides methods for treating an ocular neovascular disease in an individual. In another aspect, the present disclosure provides methods for reducing retinal fluid in the eye of an individual with an ocular neovascular disease.

In some embodiments, the ocular neovascular disease is age-related macular degeneration (AMD), wet-AMD, retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy, diabetic retinal edema, or any combination thereof. In some embodiments, the ocular neovascular disease is active choroidal neovascularization (CNV) secondary to age-related macular degeneration (AMD).

In some embodiments, the ocular neovascular disease is recurrent and/or persistent wAMD. In some embodiments, the ocular neovascular disease is active subfoveal CNV secondary to AMD. In some embodiments, the active subfoveal CNV secondary to AMD occupies ≥50% of the total lesion size. In some embodiments, the active subfoveal CNV secondary to AMD occupies ≥50% of the total lesion size with evidence of leakage on fluorescein angiogram (FA), fluid on spectral domain optical coherence tomography (SD-OCT), and/or subretinal hemorrhage on color fundus photography. In some embodiments, the active subfoveal CNV secondary to AMD occupies ≥50% of the total lesion size with evidence of leakage on fluorescein angiogram (FA), fluid on spectral domain optical coherence tomography (SD-OCT), and/or subretinal hemorrhage on color fundus photography, and the entire dimension of the lesion does not exceed 12 macular photocoagulation study disc areas. In some embodiments, the one eye and/or the contralateral eye of the individual exhibited best corrected visual acuity (BCVA) based on an ETDRS letters assessment of 78-25 (e.g., less than any of about 78, about 75, about 70, about 65, about 60, about 55, about 50, about 45, about 40, about 35, about 30, or about 25) prior to administration of the unit dose of rAAV particles of the present disclosure. In some embodiments, the one eye and/or the contralateral eye of the individual exhibited best corrected visual acuity (BCVA) based on an ETDRS letters assessment of more than any of about 5, about 10, about 15, about 20, about 25, about 30, about 35, about 40, about 45, about 50, about 55, about 60, about 65, about 70, about 75, about 80, about 85, about 90, about 95, or about 100 prior to administration of the unit dose of rAAV particles of the present disclosure.

In some embodiments, the individual had polypoidal choroidal vasculopathy (PCV) in the one eye and/or the contralateral eye prior to administration of the unit dose of rAAV particles.

In some embodiments, ETDRS letters assessment is done at about 0.5 meters, about 1 meter, about 2 meters, about 3 meters, or about 4 meters. In some embodiments, ETDRS letters assessment is done at about 4 meters.

In some embodiments, the individual received at least one prior treatment (e.g., at least one, at least two, at least three, at least four, at least 5 or more treatments) with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in about the last 12 weeks (e.g., about 3 or about 4 months) prior to administration of the unit dose of rAAV particles. In some embodiments, the individual received 2 or 3 prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during about the last 12 weeks (e.g., about 3 or about 4 months) prior to administration of the unit dose of rAAV particles to the one eye and/or the contralateral eye. In some embodiments, the individual received at least about 1, at least about 5, at least about 10, at least about 20, at least about 30, at least about 40, at least about 50, at least about 60, at least about 70, at least about 80, at least about 90, at least about 100, at least about 110, at least about 120, or more prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye. In some embodiments, the individual had a calculated anti-VEGF agent (e.g., bevacizumab, brolucizumab, ranibizumab, and/or aflibercept) injection interval in the one eye and/or the contralateral eye of about 2 weeks, about 3 weeks, 4 weeks, about 5 weeks, about 6 weeks, about 7 weeks, about 8 weeks, about 9 weeks, about 10 weeks, about 11 weeks, about 12 weeks, or more. In some embodiments, the individual had a calculated anti-VEGF (e.g., bevacizumab, brolucizumab, ranibizumab, and/or aflibercept) injection interval in the one eye and/or the contralateral eye of about 5-7 weeks, about 4-10 weeks, about 4-7 weeks, or about 4-6 weeks. In some embodiments, the individual received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye any of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, or at least about 20 days prior to administration of the unit dose of rAAV particles to the one eye and/or the contralateral eye. In some embodiments, the individual received a prior treatment with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or the contralateral eye about 7 days, about 10 days, or about 14 days prior to administration of the unit dose of rAAV particles to the one eye and/or the contralateral eye. In some embodiments, the prior treatment comprises an intraocular, subretinal or intravitreal injection with an anti-VEGF agent. In some embodiments, the anti-VEGF agent is bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept. In some embodiments, the anti-VEGF agent is aflibercept. In some embodiments, the individual received between 1 and 20 (e.g., any of 1, 2, 3, 4, 5, 6, 7, 8, 9, 10, 11, 12, 13, 14, 15, 16, 17, 18, 19, or 20) prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during the last about 12 months prior to administration of the unit dose of rAAV particles to the one eye and/or the contralateral eye. In some embodiments, the individual received about 9 or about 10 prior treatments with an anti-VEGF agent (e.g., bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept) in the one eye and/or in the contralateral eye during the last about 12 months prior to administration of the unit dose of rAAV particles to the one eye and/or the contralateral eye.

In some embodiments, the individual demonstrated a meaningful response to a prior treatment with anti-VEGF agent. In some embodiments, the anti-VEGF agent is aflibercept, a functional variant thereof, or a functional fragment thereof. In some embodiments, the anti-VEGF agent comprises a polypeptide comprising an amino acid sequence with at least about 95% identity to the amino acid sequence of SEQ ID NO: 35. In some embodiments, the individual demonstrated a meaningful response to a prior anti-VEGF treatment (e.g., aflibercept, a functional variant thereof, or a functional fragment thereof) for the ocular neovascular disease in the one eye and/or in the contralateral eye prior to administration of the unit dose of rAAV particles to one eye and/or the contralateral eye. In some embodiments, the individual is determined to have a meaningful response to a prior anti-VEGF treatment (e.g., aflibercept, a functional variant thereof, or a functional fragment thereof) for the ocular neovascular disease if a reduction of ≥30% (e.g., any of at least 30%, at least 40%, at least 50%, at least 60%, at least 70%, at least 80%, at least 90%, or 100%) of central retinal thickness (CRT) or central subfield thickness (CST) is observed any of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, or more, after the anti-VEGF treatment, compared to the central retinal thickness (CRT) or central subfield thickness (CST) at the initial diagnosis in the one eye and/or the contralateral eye. In some embodiments, the individual is determined to have a meaningful response to a prior anti-VEGF treatment (e.g., aflibercept, a functional variant thereof, or a functional fragment thereof) for the ocular neovascular disease if a reduction of ≥30% (e.g., any of at least 30%, at least 40%, at least 50%, at least 60%, at least 70%, at least 80%, at least 90%, or 100%) in central retinal thickness (CRT) or central subfield thickness (CST) is observed more than any of about 7 days, about 10 days, or about 14 days after the anti-VEGF treatment, compared to the central retinal thickness (CRT) or central subfield thickness (CST) prior to administration of the prior anti-VEGF treatment in the one eye and/or the contralateral eye.

In some embodiments, the central subfield thickness and/or central retinal thickness is determined by SD-OCT in the one eye and/or the contralateral eye. Central subfield thickness is the mean thickness of the retina across the central subfield of an ETDRS grid, a 1 mm diameter circle centered on the fovea.

In some embodiments, the individual is determined to have a meaningful response to a prior treatment with anti-VEGF agent (e.g., aflibercept, a functional variant thereof, or a functional fragment thereof) for the ocular neovascular disease if a reduction of ≥20% (e.g., any of at least 20%, at least 30%, at least 40%, at least 50%, at least 60%, at least 70%, at least 80%, at least 90%, or 100%) in central subfield thickness and/or central retinal thickness is observed compared to the central subfield thickness and/or central retinal thickness prior to administration of the prior treatment with the anti-VEGF agent. In some embodiments, the individual is determined to have a meaningful response to a prior treatment with anti-VEGF agent (e.g., aflibercept, a functional variant thereof, or a functional fragment thereof) for the ocular neovascular disease if a reduction of ≥20% (e.g., any of at least 20%, at least 30%, at least 40%, at least 50%, at least 60%, at least 70%, at least 80%, at least 90%, or 100%) in central subfield thickness and/or central retinal thickness is observed any of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, or more, after administration of the prior treatment with an anti-VEGF agent, compared to the central subfield thickness and/or central retinal thickness prior to administration of the prior treatment with an anti-VEGF agent. In some embodiments, the individual is determined to have a meaningful response to a prior treatment with an anti-VEGF agent (e.g., aflibercept, a functional variant thereof, or a functional fragment thereof) for the ocular neovascular disease if a reduction of >20% (e.g., any of at least 20%, at least 30%, at least 40%, at least 50%, at least 60%, at least 70%, at least 80%, at least 90%, or 100%) in central subfield thickness and/or central retinal thickness is observed about 7 days, about 10 days, or about 14 days after administration of the prior treatment with the anti-VEGF agent, compared to the central subfield thickness and/or central retinal thickness prior to administration of the prior treatment with an anti-VEGF agent. In some embodiments, the central subfield thickness and/or central retinal thickness is determined by SD-OCT in the one eye and/or the contralateral eye. In some embodiments, the individual is determined to have a meaningful response to a prior treatment with an anti-VEGF agent (e.g., aflibercept, a functional variant thereof, or a functional fragment thereof) for the ocular neovascular disease if normalization of CST is observed with no observable vascular exudation after the anti-VEGF treatment in the one eye and/or the contralateral eye. Normalization refers to a CST value that is normal for that class of patient (e.g., based on age, sex, etc.)

In some embodiments, the individual is determined to have a meaningful response to a prior anti-VEGF treatment (e.g., aflibercept, a functional variant thereof, or a functional fragment thereof) for the ocular neovascular disease if a reduction of ≥20% (e.g., any of at least 20%, at least 30%, at least 40%, at least 50%, at least 60%, at least 70%, at least 80%, at least 90%, or 100%) in central retinal thickness (CRT) or central subfield thickness (CST) is observed any of at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, or more, after the anti-VEGF treatment, compared to the central retinal thickness (CRT) or central subfield thickness (CST) prior to administration of the anti-VEGF treatment (e.g., aflibercept), as determined by SD-OCT in the one eye and/or the contralateral eye. In some embodiments, the individual is determined to have a meaningful response to a prior anti-VEGF treatment (e.g., aflibercept, a functional variant thereof, or a functional fragment thereof) for the ocular neovascular disease if a reduction of ≥20% (e.g., any of at least 20%, at least 30%, at least 40%, at least 50%, at least 60%, at least 70%, at least 80%, at least 90%, or 100%) in central retinal thickness (CRT) or central subfield thickness (CST) is observed about 7 days, about 10 days, or about 14 days after the anti-VEGF treatment, compared to the central retinal thickness (CRT) or central subfield thickness (CST) prior to administration of the anti-VEGF treatment, as determined by SD-OCT in the one eye and/or the contralateral eye.

In some embodiments, the individual has not received a prior treatment for an ocular neovascular disease. In some embodiments, the individual has not received a prior treatment in the one eye and/or the contralateral eye for an ocular neovascular disease. In some embodiments, the individual has not received a prior anti-VEGF treatment. In some embodiments, the individual has not received a prior anti-VEGF treatment in the one eye and/or the contralateral eye. In some embodiments, the individual has not received a prior aflibercept treatment. In some embodiments, the individual has not received a prior aflibercept treatment in the one eye and/or the contralateral eye.

In some embodiments, the ocular disease or disorder treated according to the methods described herein is diabetic macular edema. Diabetic macular edema (DME) is a swelling of the retina in diabetes mellitus due to leaking of fluid from blood vessels within the macula. The macula is the central portion of the retina, a small area rich in cones, the specialized nerve endings that detect color and upon which daytime vision depends. As macular edema develops, blurring occurs in the middle or just to the side of the central visual field. Visual loss from diabetic macular edema can progress over a period of months and make it impossible to focus clearly. Common symptoms of DME are blurry vision, floaters, double vision, and eventually blindness if it goes untreated. In some embodiments, methods and pharmaceutical compositions as disclosed herein are used to treat DME. In some embodiments, treatment of DME is assessed by measuring refraction or visual acuity (e.g., BCVA using ETDRS letters). In some embodiments, visual acuity is measured starting at a distance of about 4 meters prior to dilation of the one eye and/or the contralateral eye. In some embodiments, visual acuity is considered to be maintained if the individual loses fewer than 15 letters in the ETDRS score after administration of the unit dose of rAAV particles compared to prior to administration of the unit dose of rAAV particles. In some embodiments, treatment of DME is assessed by SD-OCT and/or OCT-A. In some embodiments, treatment of DME is assessed by measuring central subfield thickness and/or macular volume using SD-OCT. In some embodiments, treatment of DME is assessed by the number of treatments with an anti-VEGF agent (e.g., aflibercept) administered after administration of the unit dose of rAAV particles (e.g., aflibercept rescue treatments). In some embodiments, one or more aflibercept rescue treatments are administered after administration of the unit dose of rAAV particles if an increase in central subfield thickness (CST) of >50 μm occurs, assessed by SD-OCT, compared to the lower of the two CST measurements recorded prior to administration of the unit dose of rAAV particles (e.g., on Day 1) and about 3 weeks after administration of the unit dose of rAAV particles (e.g., on Week 4). In some embodiments, one or more aflibercept rescue treatments are administered after administration of the unit dose of rAAV particles if loss of >5 letters in BCVA occurs due to worsening DME disease activity compared to the higher of the two BCVA measurements recorded prior to administration of the unit dose of rAAV particles (e.g., on Day 1) and about 3 weeks after administration of the unit dose of rAAV particles (e.g., on Week 4). In some embodiments, treatment of DME is assessed using the Diabetic Retinopathy Severity Scale (DRSS), e.g., using ultra-wide field color fundus photography, compared to DRSS prior to administration of the unit dose of rAAV particles. In some embodiments, treatment of DME is assessed by the occurrence of vision threatening complications (e.g., anterior segment neovascularization, diabetic macular edema, high-risk PDR development, vitreous hemorrhage, or tractional retinal detachment). In some embodiments, vision threatening complications are assessed by ultra-wide field imaging and clinical examination. In some embodiments, the administering the unit dose of rAAV particles to the one eye and/or to the contralateral eye of the individual results in a 2-step or in a 3-step improvement in Diabetic Retinopathy Severity Scale (DRSS). In some embodiments, the administering the unit dose of rAAV particles to the one eye and/or to the contralateral eye of the individual results in a 2-step improvement in Diabetic Retinopathy Severity Scale (DRSS). In some embodiments, the administering the unit dose of rAAV particles to the one eye and/or to the contralateral eye of the individual results in a 3-step improvement in Diabetic Retinopathy Severity Scale (DRSS).

In some embodiments, the ocular disease or disorder treated according to the methods described herein is a retinal vein occlusion. Retinal vein occlusion is a blockage of the small veins that carry blood away from the retina. The retina is the layer of tissue at the back of the inner eye that converts light images to nerve signals and sends them to the brain. Retinal vein occlusion is most often caused by hardening of the arteries (atherosclerosis) and the formation of a blood clot. Blockage of smaller veins (branch veins or BRVO) in the retina often occurs in places where retinal arteries that have been thickened or hardened by atherosclerosis cross over and place pressure on a retinal vein. Symptoms of retinal vein occlusion can include a sudden blurring or vision loss in all or part of one eye.

In some embodiments, the ocular disease or disorder treated according to the methods described herein is choroidal neovascularization (CNV), also known as wet age-related macular degeneration (wAMD). Choroidal neovascularization can involve the growth of new blood vessels that originate from the choroid through a break in the Bruch membrane into the sub-retinal pigment epithelium (sub-RPE) or subretinal space, which can be a major cause of visual loss. CNV can create a sudden deterioration of central vision, noticeable within a few weeks. Other symptoms can include color disturbances, and metamorphopsia (distortions in which straight lines appears wavy). Hemorrhaging of the new blood vessels can accelerate the onset of symptoms of CNV. CNV may also include feeling of pressure behind the eye.

The advanced “wet” form (neovascular or exudative) of AMD may frequently cause a rapid and often substantial loss of central vision in patients. In the wet form of AMD, choroidal neovascularization forms and develops into a network of vessels that may grow under and through the retinal pigment epithelium. As this is accompanied by leakage of plasma and/or hemorrhage into the subretinal space, there could be severe sudden loss of central vision if this occurs in the macula. The present disclosure contemplates treatment or prevention of AMD, wet AMD. In some embodiments, methods and pharmaceutical compositions as disclosed herein are used to treat AMD.

In some embodiments, methods described herein are used to prevent or treat an ocular disease or disorder in a subject who has received prior treatment with bevacizumab, brolucizumab, ranibizumab, faricimab, abicipar pegol, conbercept, OPT-302, KSI-301, injectable sunitinib maleate (GB-102), PAN-90806 (PanOptica), and/or aflibercept. In some embodiments, methods described herein are used to prevent or treat an ocular disease or disorder that is responsive to treatment with bevacizumab, brolucizumab, ranibizumab, and/or aflibercept.

In some embodiments, the individual was diagnosed with the ocular neovascular disease at least 1 day, at least 1 week, at least 1 month, at least 2 months, at least 4 months, at least 6 months, at least 12 months, at least 18 months, at least 24 months, at least 30 months, at least 36 months, at least 42 months, at least 48 months, at least 54 months, at least 60 months, at least 66 months, at least 72 months, at least 78 months, at least 84 months, at least 90 months, 96 months, at least 102 months, at least 108 months, at least 114 months, at least 120 months, at least 126 months, at least 132 months, or more, prior to administration of the unit dose of rAAV particles to the one eye and/or the contralateral eye.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1-Low Dose ADVM-022 for the Treatment of Neovascular AMD

Intravitreal (IVT) administration of anti-vascular endothelial growth factor (VEGF) (such as aflibercept) is the standard of care for neovascular age-related macular degeneration. A shortcoming of these therapies is the need for frequent injections associated with fluctuation in central subretinal fluid ultimately impacting best corrected visual acuity. Therefore, there is an urgent need to develop new VEGF long-acting therapeutic modalities. To date, a single IVT injection of ADVM-022 has resulted in robust levels of aflibercept at therapeutic concentrations out as long as 104 weeks, as demonstrated in an ongoing, long-term clinical study, OPTIC. Both the aforementioned study and the current, non-clinical study described here utilize ADVM-022, an adeno-associated virus (AAV) gene therapy vector encoding aflibercept packaged in the chimeric AAV.7m8 capsid. ADVM-022 has been shown to reduce annual injection frequency by >80%, and in most patients eliminate the need for any additional injections of anti-VEGF. Previous non-clinical studies demonstrated a nearly flat dose response suggesting a lower dose could also yield efficacious aflibercept levels.

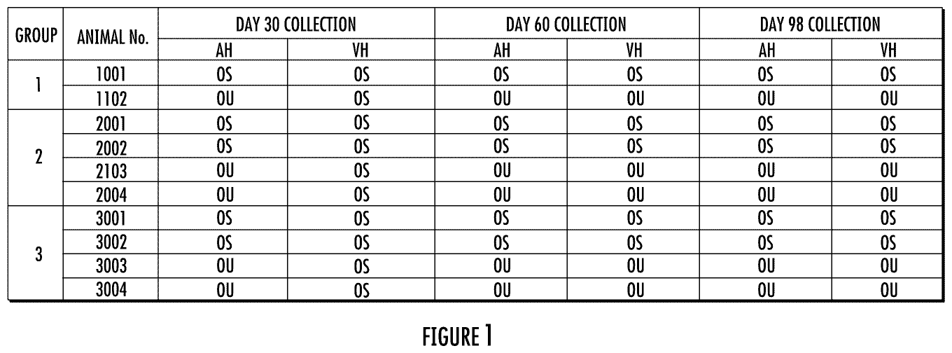

To assess whether lower doses of ADVM-022 provide relevant ocular aflibercept levels we performed a good laboratory practice (GLP) toxicology study using non-human primates (NHP). In this study, NHP subjects were administered bilateral IVT ADVM-022 at either 3E10 vg/eye or 1E11 vg/eye (n=4/group), or vehicle (n=2/group). Vitreous (VH) and aqueous humors (AH) were collected for aflibercept quantification on days 30, 60 and 98 post dose. See FIG. 1. In addition, ophthalmic examinations and tonometry were performed once pretreatment, and throughout the study. Electroretinography (ERG) and optical coherence tomography (OCT) were performed once pretreatment, and at weeks 4 and 12.

An ELISA detection assay was used to measure Aflibercept levels in non-human primate aqueous and vitreous humors. Briefly, 1 μg/mL of capture reagent (rhVEGF165, R&D Systems, Cat #293-VE-500) was diluted in coating buffer (R&D System, Cat #DY006) and coated onto a 96-well microtiter plate overnight at 2-8° C. The next day, the plate was washed with wash buffer (KPL, Cat #5150-0011) and blocked with protein-free blocking buffer (Pierce, Cat #37572) for at least 60 minutes at room temperature. This was followed by a wash step. Aflibercept standards (Eylea, Regeneron, Cat #NDC 61755-005-55) ranging from 10 ng/ml to 0.078 ng/ml and samples diluted to at least 1:50 in reagent diluent (1×PBS and 1% BSA, Pierce, Cat #A9576-50 mL) were loaded on the plate and incubated for 2 hours at room temperature with shaking at 200 rpm followed by a wash step. Subsequently, a detection antibody, horse radish peroxidase- (HRP-) conjugated mouse-anti-human IgG Fc (Jackson ImmunoResearch, Cat #209-035-098) was added to the plate and incubated for 2 hours at room temperature with shaking at 200 rpm and then washed. The plate was developed using SuperSignal ELISA Pico Chemiluminescent HRP substrate (ThermoFisher, Cat #37069) and read on a SpectraMax M3 plate reader (Molecular Devices). Luminescence is plotted versus aflibercept concentration.

Administration of ADVM-022 at both doses resulted in meaningful aflibercept levels with peak aflibercept levels at approximately 4-weeks post dose. Mean peak aflibercept levels at the 3E10 vg/eye dose were 0.942 μg/mL for VH and 0.535 μg/mL for AH. At the 1E11 vg/eye dose, mean peak aflibercept levels were 1.339 μg/mL for VH and 0.834 μg/mL for AH. These peak levels were comparable with aflibercept levels observed in previous studies utilizing higher doses of ADVM-022. See Tables 2 and 3.

Overall IVT administration of ADVM-022 in NHP subjects was well tolerated. No adverse systemic clinical signs were observed during the 3-month study. In life observations were dose-dependent and limited to non-adverse slight to mild dose-dependent ocular inflammation characterized by pigment and cells in the VH. No abnormalities of the anterior segment or lens were found, and no iris changes were observed by transillumination. Microscopic findings were limited to minimal mononuclear cell infiltrations, considered non-adverse based on minor severity, in agreement with absence of abnormal findings in ocular physiology and function in animals.

In summary, the findings from the non-clinical NHP study suggest administration of ADVM-022 at doses as low as 3E10 vg/eye (human equivalent dose of 6E10 vg/eye) may provide therapeutic levels of aflibercept in the clinic.

### Example 2 Biodistribution Analysis

NHP tissues collected at termination of the in life part of the study intended for vector genome DNA analysis were snap frozen on dry ice and stored at −80° C.

During collection of tissue samples for mRNA analysis, instruments were wiped with RNaseZap™ Wipes from ThermoFisher or similar before use. Tissue samples collected for mRNA analysis were incubated in RNAlater at (2 to 8° C.) for 24 hours, in RNAse and DNAse free tube and then the RNAlater was discarded. After that tissues were transferred to dry ice or a freezer set to maintain −80° C.

Tissues for the vector DNA and mRNA biodistribution analysis included: ciliary body with iris (right and left), cornea (right and left), choroid (right and left), lens (right), optic nerve (right and left), lateral geniculate nucleus (left), optic chiasm (left part), optic tract (left), spleen, salivary glands (right and left), lymph nodes (left mandibular and mesenteric), eyelid (right and left), liver (left lateral lobe), heart (left ventricular), kidney (right and left), lung (left caudal lobe), testis (right and left), cerebellum (left), and visual cortex (right and left). For vector genome DNA analysis, whole blood was collected to venipuncture to K2EDTA tubes and frozen over dry ice or in a freezer set to maintain-80° C., within 60 minutes of blood collection. For the RNA analysis, blood samples were collected to RNAprotect Animal Blood Tubes (QIAGEN or similar). Blood samples were placed on dry ice within 60 minutes of collection and stored in a freezer set to maintain −80° C. until analysis.

Vector DNA biodistribution was measured by quantitative polymerase chain reaction (qPCR). The number of vector DNA copies were inferred from a copy number standard curve and calculated per microgram (μg) of tissue DNA. In brief, Total DNA was extracted from Cynomolgus monkey tissue and blood using QIAsymphony DSP DNA mini kit on QIAsymphony. qPCR reactions were performed on 96-well plates. Each plate was run with a standard curve, a set of QC samples, and study samples. The forward primer (ADVM-022F) sequence was 5′-GAGTGACAATGACATCCACTTTG-3, (SEQ ID NO: 42). The reverse primer (ADVM-022R) sequence was 5′-AGACTCCAGTATCCCAGTATGA-3′ (SEQ ID NO: 43). The ADVM-022 probe sequence (ADVM-022P) was 5′-6FAM-AAACTTGGACCTGGGAGTGGACAC (SEQ ID NO: 44)-MGBNFQ-3′. The standard curve was run in 3 replicates for the standard points at 106, 105, 103, 102, 50, 20, 10, and 0 copies per reaction. The 0 copy standards, containing only matrix, were included to confirm reagent purity. Triplicate QC samples were prepared at 105 (QC high), 104 (QC mid), and 103 (QC low) copies/reaction in a background of 1,000 ng Cynomolgus monkey matrix DNA per reaction. When possible, the tissue DNA samples were tested at 1,000 ng per reaction and the blood DNA samples were tested at 100 ng per reaction. The blood DNA was tested at a lower reaction based on previous observations that 1,000 ng of blood DNA per reaction inhibited qPCR. If it was not possible to load the amounts specified above for a specific sample (because the DNA concentration was too low or the sample volume was limiting), a smaller amount of sample DNA was analyzed. All sample reactions were run in triplicate, and the third reaction was spiked with 200 copies of pAVA210 DNA to evaluate potential qPCR inhibition. If qPCR inhibition was observed as shown by the measured value in the third spiked well at less than 110 copies of the target DNA, the sample DNA were reanalyzed at lower amount.

### Example 3. Transgene mRNA Expression Analysis

Transgene mRNA expression analysis was performed using RTqPCR method. In brief, RNA was purified from Cynomolgus monkey tissue samples using test site operation procedures, and cDNA was synthetized using the Maxima H Minus cDNA Synthesis Master Mix. TaqMan-based RT-qPCR reactions were performed on 96-well plates. Each plate was run with a standard curve, 2 sets of QC samples, and study samples. The standard curve was run in 3 replicates using serial dilutions of pAVA266-Flip pFB-C11-AfliCo plasmid, at 107, 106, 104, 103, 50, 25, 10, and 0 copies per reaction. The 0 copy standards were included to confirm reagent purity. Duplicate QC samples were prepared at 106 (QC high), 104 (QC mid), and 103 (QC low) copies/reaction of pAVA266-Flip pFB-C11-AfliCo, mRNA DNA per reaction. The analyses utilized following set of primers. The forward primer AfliCo-F1 sequence was 5′-TGCCCGCACCTATTGAAAAG-3′ (SEQ ID NO: 45). The reverse primer AfliCo-R1 sequence was 5′-GAGGCTCGCGAGGTTGAC-3′ (SEQ ID NO: 46). The AfliCo-P1 Probe sequence was 5′-6-FAM-CATCTCCAAGGCCAAG-MGBNFQ-3′ (SEQ ID NO: 47). When possible, the tissue cDNA samples were tested at 10 ng per reaction. If it was not possible to load the amount specified above for a specific sample (because the DNA concentration was too low or the sample volume was limiting), a smaller amount of sample DNA was analyzed. As a control to monitor the vector DNA contamination in the isolated RNA samples, each RNA sample was analyzed in a single well by qPCR at 10 ng of total RNA per well. As another control to monitor overall RNA integrity, relative quantitation of the monkey housekeeping gene Hprt1 cDNA, was also performed in duplicate qPCR wells.

### Example 4. Ocular Compartment Analysis

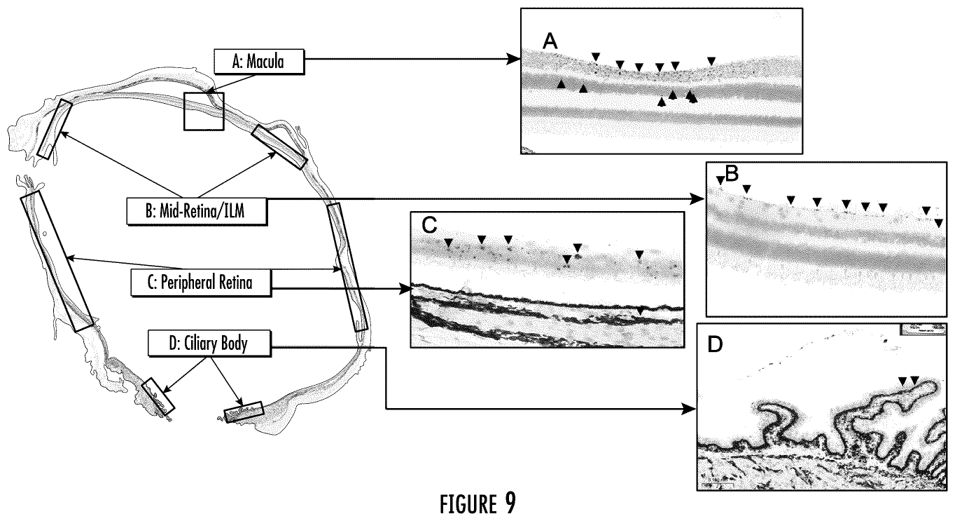

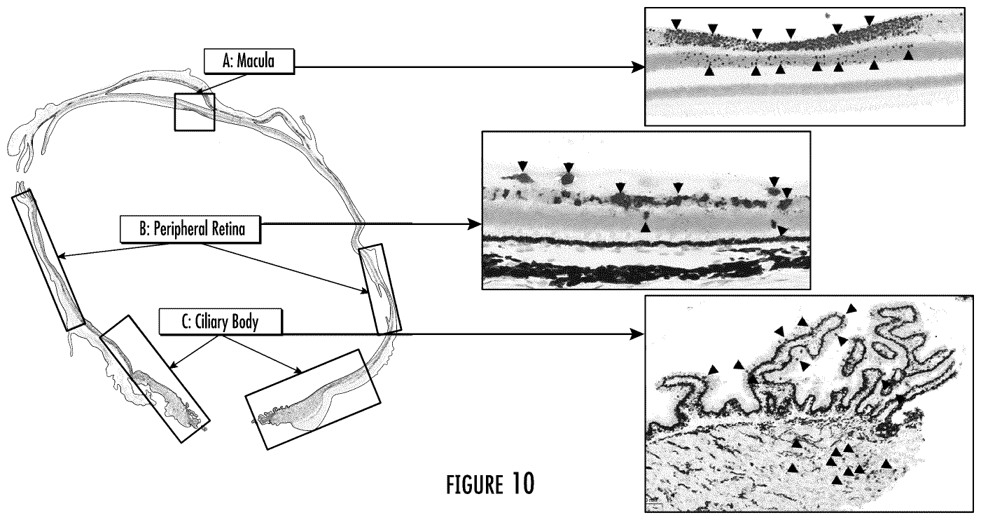

ADVM-022 vector genome DNA and transgenic mRNA microscopic distribution in ocular compartments were evaluated using BaseScope In-Situ Hybridization Methods (ISH). For the analysis, upon eye enucleation, the cornea, iris and lens were removed from the globes. The remaining posterior segment and isolated anterior structures were fixed in 10% neutral buffered formalin for 24 hours and stored in 70% ethanol up to 72-hours. The tissues were prepared for paraffin embedding using vacuum infiltration processing and placed into paraffin blocks and stored at 4° C. until sectioning. Transverse sections of the posterior segment were collected through the macula at 5-μm thickness and mounted on charged slides. Transverse sections of the isolated anterior structures were collected at 5-μm thickness and mounted on charged slides. Slides were stored at 4° C. until shipment to Advanced Cell Diagnostics (ACD). Ten sequential slides were shipped to ACD for subsequent in-situ hybridization (ISH) analysis. ISH was performed using the BaseScope LS Red Reagent Kit (Cat. No. 323600) following optimized pretreatment conditions which included epitope retrieval of 15 minutes at 88° C., and protease III treatment for 15 minutes at 40° C. ISH labeling of ADVM-022 vector genome DNA was performed using a 1-ZZ paired probe to the sense of the CMV promoter. Results from one such experiment are shown in FIG. 9. Labeling of ADVM-022 derived Aflibercept mRNA was performed using a 1-ZZ paired probe complementary to the splicing junction, generated after the intron has been spliced from mRNA during maturation, which allows for the detection of ADVM-022 expressed mRNA without DNA interference. Results from one such experiment are shown in FIG. 10. Counterstain of Gill's hematoxylin for 2 minutes at RT was performed after probe, amplification, and chromogen steps in the LS kit assay.

### Example 5. A Phase 2 Study to Assess Safety and Efficacy of AAV2.7m8-Aflibercept in Anti-VEGF Treatment-Experienced Patients with Neovascular (Wet) Age-Related Macular Degeneration (nAMD)

This Example describes a Phase 2 study to assess the safety, tolerability, and efficacy of AAV2.7m8-aflibercept, an rAAV vector containing the VEGF inhibitor aflibercept and the AAV2.7m8 protein capsid, in anti-VEGF treatment-experienced patients with neovascular (wet) age-related macular degeneration (nAMD).

### I. Study Objectives and Endpoints

This study is designed to evaluate the safety, tolerability and efficacy of a single intravitreal injection of AAV2.7m8-aflibercept at two dose levels (6×10¹⁰ vg/eye and 2×10¹¹ vg/eye) in patients with nAMD that have persistent retinal fluid and a demonstrated response to aflibercept. The objectives and endpoints of this study are provided below.

### Primary Objectives and Endpoints

The primary objectives of this study include:
To assess the efficacy, tolerability and safety of a single intravitreal (IVT) injection of AAV2.7m8-aflibercept in anti-VEGF treatment-experienced patients with neovascular (wet) age-related macular degeneration (nAMD).

The primary endpoints of this study include:
Incidence and severity of ocular and non-ocular adverse events.
Mean change in BCVA using an ETDRS visual acuity chart from Baseline at Week 50/end of study.

### Secondary Objectives and Endpoints

The secondary objectives of this study include:
To assess the durability of a single IVT injection of AAV2.7m8-aflibercept.
To evaluate the effect of AAV2.7m8-aflibercept on central subfield thickness (CST).
To evaluate the effect of AAV2.7m8-aflibercept on Best Corrected Visual Acuity (BCVA) using an ETDRS visual acuity chart.
To assess the effectiveness of prophylactic corticosteroid treatment regimens on minimizing post-prophylactic inflammation events.

The secondary endpoints of this study include:
Percentage of participants who lose/gain at least 5, 10, or 15 letters in BCVA compared with Baseline through Week 50.
Mean change in BCVA from Baseline at Week 26.
Percentage of participants who are supplemental aflibercept injection-free through Week 50.
Percent reduction in mean rate of anti-VEGF injections over one year relative to the number of anti-VEGF injections received in the year prior to Baseline.
Mean change in central subfield thickness (CST) from baseline through Week 26 and Week 50.
Percentage of participants without CST fluctuations>50 μm through Week 50.
Percentage of participants without post-prophylactic inflammation through Week 50.

For assessment of secondary endpoints, baseline measurements are based on pre-injection values at the Screening visit in which aflibercept is administered.

### II. Study Population

The study population includes male and female participants, 50 years or older, diagnosed with active choroidal neovascularization (CNV) secondary to age-related macular degeneration (AMD) and have a history of responsiveness to anti-VEGF treatment.

Prior to the Screening visit (Baseline), eligible participants have received: a minimum of 2 anti-VEGF injections in the previous 4 months, anti-VEGF injections for no more than 3 calendar years (36 months) prior to Baseline, and no anti-VEGF injection in the 28 days prior to receiving aflibercept at Baseline.

### Inclusion Criteria

Individuals that meet the following criteria are included in this study:
Male or female participants, age≥50 years.
Have a central subfield macular thickness of at least 275 μm in the study eye at the Screening visit (Baseline) as assessed by SD-OCT, confirmed by the Central Reading Center (CRC) and agreed upon by the Investigator.
The study eye at the Screening visit (Baseline) has current evidence of active primary or recurrent subfoveal CNV or CNV with subfoveal involvement with fluid, as assessed by Spectral Domain Optical Coherence Tomography (SD-OCT) and confirmed by the Central Reading Center (CRC) and agreed upon by the Investigator.
Received a minimum of 2 anti-VEGF injections in the 4 months prior to the Screening visit (Baseline), received anti-VEGF injections for no more than 3 calendar years (36 months) prior to the Screening Visit, and no anti-VEGF injection in the 28 days prior to receiving aflibercept at Baseline.
Vision of the study eye at Baseline:
   BCVA in the range of 25-78 ETDRS letters, inclusive (approximate Snellen equivalent visual acuity range of 20/32-20/320).
Vision of the non-study eye at Baseline:
   BCVA≥50 ETDRS letters (approximate Snellen equivalent of 20/100 or better).
Demonstrates a meaningful anatomic response 7 to 14 days after IVT aflibercept injection administered at the Screening visit (Baseline), as confirmed by the CRC, agreed upon by the Investigator, and defined as:
   Reduction from the Screening visit (Baseline) in CST by ≥20% as assessed using SD-OCT, or
   Reduction of 30% in thickest paracentral subfield within ETDRS 3 mm grid, or
   No anatomical evidence of SRF or IRF if fluid is present at Baseline by clinical examination or SD-OCT.

### Exclusion Criteria

Individuals that meet any of the following general criteria are excluded from the study:
Serum anti-AAV.7m8 neutralizing antibody titer levels of 1:125 or higher determined from blood draw during Screening period (Day −21 to Day −14).
History of other disease, metabolic dysfunction, physical examination finding, or clinical laboratory findings giving reasonable suspicion of a condition that contraindicates the use of AAV2.7m8-aflibercept, contraindicates the use of systemic prednisone for 10 weeks, compromises the participant's ability to comply with the planned study activities, or that might affect the interpretation of the results of the study or render the participant at high risk for treatment complications in the opinion of the Investigator.
Ocular or periocular infection (e.g., conjunctivitis, chalazion, significant blepharitis), or intraocular inflammation (grade trace or above by Standardization of Uveitis Nomenclature [SUN] criteria), in either eye within 1 month prior to or at the Randomization visit (Day −7). Mild anticipated post-operative inflammation subsequent to the aflibercept injection administered at the Screening visit that resolves is acceptable.
Received any prior gene therapy at any time or any investigational treatment or medical device within 3 months of the Screening visit or 5 half-lives of the investigational medicinal product (whichever is longer). Observational studies involving over-the-counter vitamins, supplements, or diets are not exclusionary.
Evidence of poorly controlled diabetes or a hemoglobin A1c (HbA1c)≥7.0% within the Screening period.
History or evidence of significant uncontrolled concomitant disease within 6 months of the Screening Visit, such as cardiovascular disease, hypertension (defined as blood pressure systolic over 180 mmHg or diastolic over 100 mmHg), or nervous system, pulmonary, renal, hepatic, endocrine, or gastrointestinal disorders.
History of allergy to aflibercept, corticosteroid, or fluorescein dye or sodium fluorescein used in angiography. Mild allergy amenable to treatment is allowable.
Any history of ongoing bleeding disorders or International Normalized Ratio (INR)>3.0. The use of aspirin or other anticoagulants (e.g., Factor Xa inhibitors) is not an exclusion.
Use of systemic steroids or immunosuppressive treatments, or any systemic anti-VEGF therapy within 3 months prior to the Randomization visit (Day −7).
Any febrile illness within 1 week prior to the Randomization visit (Day −7).
History of malignancy within the last 5 years except for the following adequately treated:
   Local basal cell carcinoma of the skin.
   Carcinoma in situ of the cervix or breast.
   Papillary, noninvasive bladder cancer.
   Prostate cancer Stage 1 and 2 for which observation is clinically indicated with stable prostate-specific antigen for 6 months.
   Any other cancer that has been in complete remission for at least 2 years or considered surgically cured.
History within the 12 months prior to the Screening Visit or evidence of renal dysfunction (i.e., creatinine clearance≤50 mL/min) or hepatic dysfunction (i.e., AST or ALT≥2.5×ULN) at Screening.
Positive for human immunodeficiency virus (HIV) infection or hepatitis B or C at Screening (unless having received a documented cure for hepatitis C), or history or documented evidence of the following systemic or ocular bacterial or viral infections: SARS-COV-2 2019 (COVID-19; No history of COVID-19 infection as confirmed by PCR analysis within the past 3 months and no vaccination with a COVID-19 vaccine within the 6 weeks prior to or 6 weeks after the Randomization Visit (Day-7)), syphilis, or known to be positive (in either eye) for ocular herpes simplex virus (HSV), varicella-zoster virus (VZV), cytomegalovirus (CMV) infection including viral uveitis, retinitis or keratitis.

Individuals that have history or evidence of any of the following ocular criteria in the study eye are excluded from the study:
Evidence of subretinal hemorrhage that is either 50% or more of the total lesion area, or if the blood is under the fovea and is ≥one disc area in size as confirmed by the CRC at Screening (if blood is under the fovea, fovea must be surrounded 270 degrees by visible CNV).
Evidence of scar or macular fibrosis making up ≥50% of the total lesion area, atrophy, or other structural damage in the center of the fovea as confirmed by the CRC at Screening.
Vascularized pigmented epithelial detachment (PED) height>350 μm in the macula, as confirmed by the CRC at Screening.
History or evidence of an intraocular condition that reduces visual acuity or confounds assessment of the fundus (e.g. anterior segment neovascularization, significant vitreous hemorrhage, fibrovascular proliferation, or tractional retinal detachment) as determined by Investigator on clinical examination or SD-OCT at the Screening visit or the Randomization visit (Day −7).
History or evidence of macular or retinal disease other than nAMD, including structural abnormalities at the fovea that reduces visual acuity or confounds assessment of the macula (e.g., DME, RVO, dense hard exudates, pigment abnormalities, foveal atrophy, vitreomacular traction or epiretinal membrane that induces retinal striae within 1 mm of the foveal center, macular hole [Gass Stage 2 and above], central serous chorioretinopathy, suspected retinal angiomatous, proliferation, polypoidal choroidal vasculopathy, ocular histoplasmosis, trauma, pathologic myopia, or vascular disease in the eye [benign conditions of the vitreous or peripheral retina are non-exclusionary]), as determined by Investigator on clinical examination or confirmed by the CRC in agreement with the Investigator at Screening.
History or evidence of significant intraocular disease (other than nAMD), or media opacity that could compromise vision or reduce the potential for visual improvement during the course of the study, require medical or surgical intervention, and/or preclude proper visualization or imaging of the retina (e.g., central corneal scarring, corneal dystrophy, scleromalacia, or significant cataract including posterior subcapsular cataract or nuclear sclerotic cataract grade 2+ or worse), as determined by Investigator on clinical examination or SD-OCT at the Screening visit or the Randomization visit (Day −7).
Retinal detachment (with or without repair), or retinal pigment epithelium rip/tear at the Screening visit or the Randomization visit (Day −7).
Uncontrolled ocular hypertension or glaucoma at the first Screening visit or at the Randomization visit (Day-7) (e.g., Intraocular pressure [IOP]≥23 mmHg despite treatment with anti-glaucoma medication), cup/disc ratio>0.7, history of IOP elevation related to topical or local ocular corticosteroid administration, or current use of 2 or more IOP lowering medications at the Screening Visit or the Randomization Visit (Day −7) (combination IOP lowering medication counts as two IOP lowering medications and is disallowed).
Aphakia and/or complete or partial absence of posterior capsule at the Screening Visit or the Randomization Visit (Day −7), or cataract extraction or yttrium aluminum garnet (YAG) laser capsulotomy in the study eye within 3 months prior to the Randomization Visit (Day −7) (partial absence of the posterior capsule due to YAG laser capsulotomy performed >3 months prior to Day −7 is acceptable).
Trabeculectomy or glaucoma shunt, or minimally invasive glaucoma surgery, penetrating keratoplasty, endothelial keratoplasty, or therapeutic ocular radiation within 3 months of Screening.
Prior treatment with photodynamic therapy or retinal laser for the treatment of nAMD.
Any history of vitrectomy or any other vitreoretinal surgery within 3 months prior to the Randomization visit (Day −7).
Refractive surgery within 3 months of Screening.
Anticipated need for intraocular surgery within the next year in the opinion of the Investigator.
Intraocular or periocular steroid treatment for any ocular condition (e.g., Triesence™, Iluvien®, or Ozurdex®) within 6 months prior to the Randomization visit (Day −7).

### III. Study Design

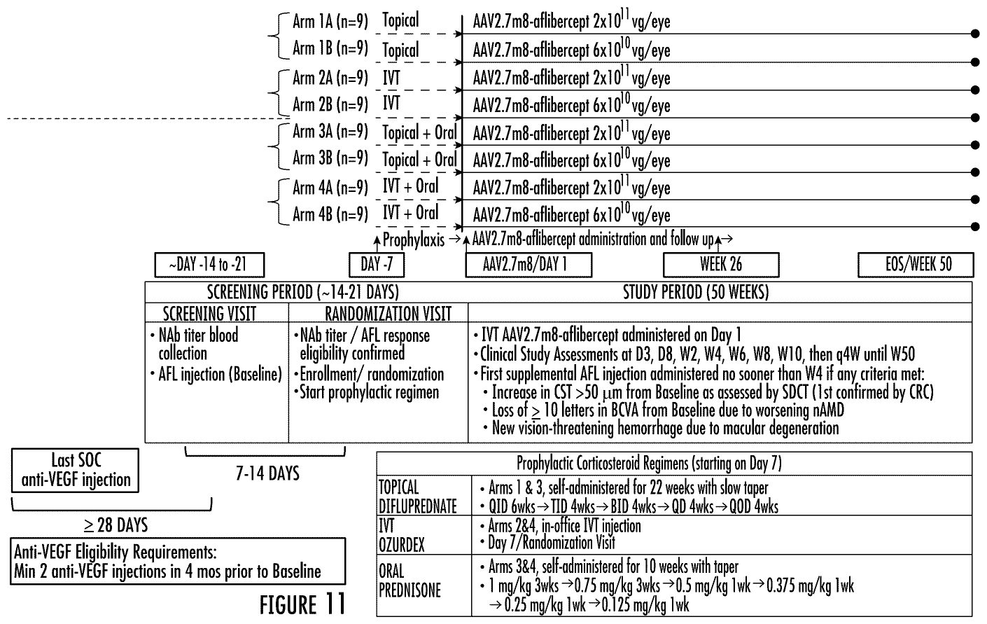

This study is a Phase 2, multi-center, randomized, double-masked, parallel group study evaluating two dose levels of AAV2.7m8-aflibercept (2×10¹¹ vg/eye [A] or 6×10¹⁰ vg/eye [B]) accompanied by evaluation of the safety and anti-inflammatory effectiveness of four prophylactic corticosteroid treatment regimens (topical [1], intravitreal [2], topical+oral [3], or intravitreal+oral [4]) (Table 1). Eligible study participants are randomized in a 1:1:1:1:1:1:1:1 ratio. Only one eye per participant is selected as the study eye. If both eyes are eligible the eye with the worse BCVA at Day −7 is the study eye. If both eyes have the same BCVA, the study eye is the right eye. See, FIG. 11 for a diagram of the study design.

All randomized study participants have study visits at Day 1, Day 3, Day 8, Week 2, Week 4, Week 6, Week 8, Week 10, and then every 4 weeks through Week 50. The primary analysis is performed at Week 50/EOS. All participants that complete the study through Week 50 are requested to take part in a separate long-term extension study with continued safety monitoring.

TABLE 1
Study Treatment Arms

| AAV2.7m8- | Study Arm | Prophylactic Corticosteroid |
|---|---|---|
| aflibercept Dose | (No. Randomized) | Regimen |
| 2 × 10¹¹ vg/eye in | Arm 1A (n = 9) | Topical ^{a} |
| 0.05 mL | Arm 2A (n = 9) | IVT Ozurdex ® ^{b} |
| | Arm 3A (n = 9) | Topical ^{a} + Oral ^{c} |
| | Arm 4A (n = 9) | IVT Ozurdex ® ^{b} + Oral ^{c} |
| 6 × 10¹⁰ vg/eye in | Arm 1B (n = 9) | Topical ^{a} |
| 0.05 mL | Arm 2B (n = 9) | IVT Ozurdex ® ^{b} |
| | Arm 3B (n = 9) | Topical ^{a} + Oral ^{c} |
| | Arm 4B (n = 9) | IVT Ozurdex ® ^{b} + Oral ^{c} |

^{a} Topical is Durezol 0.05% difluprednate ophthalmic emulsion eyedrops.
^{b} Ozurdex ® is dexamethasone intravitreal implant, 0.7 mg.
^{c} Oral is oral prednisone tablets, USP.

### Screening and Randomization

Eligible participants received their last standard of care (SOC) anti-VEGF injection at least 28 days prior to receiving the IVT aflibercept injection at Baseline.

Eligibility for randomization into the study period involves two or more study visits and may last up to 21 days prior to administration of AAV2.7m8-aflibercept at Day 1. At the Screening Visit (one or more visits between Day −21 and Day −14) blood is collected for the purposes of assessing neutralizing antibody (nAb) titers and blood chemistries, and a single intravitreal injection of aflibercept (2 mg in a 0.05-mL intravitreal injection) is administered for the purpose of assessing a meaningful anatomic response to aflibercept anti-VEGF treatment. The Screening Visit at which aflibercept is administered is considered Baseline and occurs at least 28 days after the participant's last standard of care (SOC) anti-VEGF injection. Evidence of sufficient intraretinal and/or subretinal fluid at Baseline that, in the opinion of the Investigator, would justify a 50 μL intravitreal injection of aflibercept to treat the active primary or recurrent subfoveal choroidal neovascularization (CNV) or CNV with subfoveal involvement with fluid is required. Approximately 7-14 days after the Screening Visit, eligibility by nAb titer and IVT aflibercept response is confirmed at the Randomization Visit (Day −7). Potential participants with nAb titers of 1:125 or higher and/or no meaningful anatomic response to IVT aflibercept ˜7-14 days after receiving the aflibercept injection at the Screening Visit are excluded.

A meaningful anatomic response is determined if any of the following three criteria are met at the Randomization Visit, as confirmed by Central Reading Center (CRC) and agreed upon by the Investigator:
Reduction from the Screening visit (Baseline) in CST by >20% as assessed by SD-OCT;
Reduction of 30% in thickest paracentral subfield within ETDRS 3 mm grid; or.
No anatomical evidence of intraretinal fluid (IRF) or subretinal fluid (SRF) if fluid is present at baseline by clinical examination or SD-OCT.

Eligible participants based on nAb titer and anatomic response to aflibercept are randomized to one of the 8 treatment arms (see, Table 1). The identity of the dose assignment is concealed by identical appearance, packaging, labeling and schedule of administration to mask the participant, the Investigator, and clinical staff.

### Prophylactic Corticosteroid Therapy

Randomized participants begin their assigned prophylactic corticosteroid regimen at the Randomization Visit/Day −7 (i.e., 7 days prior to AAV2.7m8-aflibercept dosing on Day 1).

Prophylactic corticosteroid therapy to be evaluated with each AAV2.7m8-aflibercept dose includes either a 22-week topical difluprednate regimen (Durezol) (Arms 1A & 1B), IVT dexamethasone implant (Ozurdex®) applied as a single in-office injection (Arms 2A & 2B), a combination of the 22-week topical difluprednate regimen with a 10-week oral prednisone regimen (Arms 3A & 3B), or a combination of IVT Ozurdex® applied as a single in-office injection with a 10-week oral prednisone regimen (Arms 4A & 4B).

Participants assigned to treatment Arm 1 (Arms 1A & 1B) and Arm 3 (Arms 3A & 3B) self-administer topical difluprednate 0.05% (Durezol® USPI) eye drops daily for 22 weeks with a slow taper. The taper ends with a self-administered dosing frequency of 1 drop every other day for the last 4 weeks (Table 2).

TABLE 2
Topical Difluprednate Prophylactic Regimen.

| Study Days | Total Number | Drops Administered |
|---|---|---|
| (No. of weeks) | of Days | per Day |
| Days-7 to 35 (6 weeks) | 42 | 4 times |
| Days 36 to 63 (4 weeks) | 28 | 3 times |
| Days 64 to 91 (4 weeks) | 28 | 2 times |
| Days 92 to 119 (4 weeks) | 28 | 1 time |
| Days 120 to 147 (4 weeks) | 28 | 1 time every other day |
| Day 148 | 0 | STOP—if no signs of |
| | | inflammation |

Tapering of corticosteroid eye drop administration frequency is only reduced according to the taper described in Table 2, and only in the absence of active inflammation. If corneal complications from use of topical difluprednate occur, consult with Medical Monitor. For participants who experience an elevation in IOP secondary to difluprednate use, focus is on utilizing IOP lowering medications to control IOP while maintaining the difluprednate dosing schedule. If this is not possible, the Medical Monitor is consulted.

Participants assigned to treatment Arm 2 (Arms 2A & 2B) and Arm 4 (Arms 4A & 4B) receive an in-office IVT injection of Ozurdex® (dexamethasone intravitreal implant, 0.7 mg) at Day −7. Intravitreal injection of Ozurdex® is administered after all other study-required assessments have been completed.

It is recognized that IVT injection of Ozurdex® (like all IVT injections) may result in acutely elevated intraocular pressure (IOP). To monitor participant safety, a post-injection safety assessment is performed immediately after administration of prophylactic IVT Ozurdex® implant. Treatment of post-injection elevated IOP, if required, is per Investigator discretion. Furthermore, in certain steroid-responsive individuals, elevated IOP may be encountered weeks to months after injection. IOP is monitored as part of all post-injection follow-up visits. Elevated intraocular pressure is treated at the discretion of the Investigator. The Medical Monitor is consulted in cases where the IOP cannot be controlled with routine use of IOP-lowering medications.

Participants assigned to treatment Arm 3 (Arms 3A & 3B) and Arm 4 (Arms 4A & 4B) self-administer oral prednisone daily at a dose based on body weight at the Screening visit for 10 weeks according to the schedule below (Table 3).

TABLE 3
Oral Prednisone Dosing Regimen.

| | | Daily Dose for Day-7 to Day 1, Week 1 |
|---|---|---|
| Body Weight at the | | Week 2, Week 3, Week 4, Week 5, |
| Screening Visit | | Week 6, Week 7, Week 8, and Week 9^{a} |
| ≥80 | kg | 80 mg, 80 mg, 80 mg, 60 mg, 60 mg, 60 mg, |
| | | 40 mg, 30 mg, 20 mg, 10 mg^{a} 5 mg^{a} |
| 70-79 | kg | 70 mg, 70 mg, 70 mg, 50 mg, 50 mg, 50 mg, |
| | | 40 mg, 30 mg, 20 mg, 10 mg^{a} 5 mg^{a} |
| 60-69 | kg | 60 mg, 60 mg, 60 mg, 45 mg, 45 mg, 45 mg, |
| | | 30 mg, 20 mg, 10 mg, 5 mg |
| 50-59 | kg | 50 mg, 50 mg, 50 mg, 35 mg, 35 mg, 35 mg, |
| | | 25 mg, 15 mg, 10 mg, 5 mg |
| 40-49 | kg | 40 mg, 40 mg, 40 mg, 30 mg, 30 mg, 30 mg, |
| | | 20 mg, 15 mg, 10 mg, 5 mg |

^{a}For participants in the body weight range ≥70 kg, Week 9 dosing includes taper to 10 mg/day for 4 days followed by 5 mg/day for 3 days

### Study Treatment

On Day 1, eligible participants receive an injection of AAV2.7m8-aflibercept administered intravitreally in the study eye (7 days after starting their assigned prophylactic corticosteroid regimen). The identity of the AAV2.7m8-aflibercept dose assigned to a participant is masked to the Investigator, clinical staff, and the participant. If the participant receives an Ozurdex® injection, the AAV2.7m8-aflibercept ocular injection is in a quadrant at least 90° from the quadrant in which Ozurdex® is injected. Participants return for study visits on Days 3 and 8, Weeks 2, 4, 6, 8, 10, and every 4 weeks thereafter through Week 50, for clinical evaluation.

### Supplemental Aflibercept Treatment

Participants are assessed for the need of supplemental aflibercept treatment starting at Week 4. The first supplemental aflibercept injection (if required) occurs no sooner than Week 4 and requires any of the following criteria to be met:
Increase in CST>50 μm from Baseline, as assessed by SD-OCT (confirmed by CRC);
Loss of >10 letters in BCVA (using an ETDRS visual acuity chart) from Baseline due to worsening nAMD; or
Vision-threatening hemorrhage due to macular degeneration.

The above criteria are considered prior to administration of any supplemental aflibercept treatment (initial and subsequent injections). Central Reading Center confirmation of meeting the CST criteria is not required prior to administration of a supplemental aflibercept treatment after the initial supplemental injection. Aflibercept is not injected in eyes with active inflammation.

### Study Duration

Total study duration is up to 53 weeks, including a 3-week screening period prior to Day 1, and a 50-week study period following dosing with AAV2.7m8-aflibercept. The primary analysis is performed at Week 50/end of study.

All participants who complete the study through Week 50 may take part in a long-term extension study with continued safety monitoring.

### IV. Study Assessments and Procedures

### Efficacy Assessments and Procedures

Efficacy of AAV2.7m8-aflibercept is evaluated based on the following assessments:
Refraction and BCVA as measured using an ETDRS visual acuity chart.
Central subfield thickness as measured by a central reading center using SD-OCT images.
Number of supplemental aflibercept injections post AAV2.7m8-aflibercept administration.
Presence of IRF.
Presence of SRF.
Aqueous humor (AH) concentration of total aflibercept.

### Refraction and Best-Corrected Visual Acuity

Refraction and BCVA are measured using an ETDRS visual acuity chart at a starting distance of 4 meters, prior to dilating eyes or any examination requiring contact with the eye, or administration of ocular treatment.

### Spectral Domain Optical Coherence Tomography

SD-OCT imaging assessments are performed using the same equipment for all study visits of a participant. SD-OCT images are submitted to the CRC for determination of participant eligibility at Screening, as well as for determination of need for supplemental aflibercept as required throughout the Study Period.

### Safety Assessments

The safety of AAV2.7m8-aflibercept is assessed through the collection of adverse events (AEs), vital signs, physical examinations, ocular examinations, imaging, and laboratory evaluations.

### Ophthalmic Examination

Components of the full ophthalmic examination performed in this study are described in Table 4.

TABLE 4
Full Ophthalmic Examination.

| Test | Test Parameters |
|---|---|
| Slit-lamp | External examination of the eye and adnexa, |
| Biomicroscopy | screening of eyelid/pupil responsiveness, |
| | slit-lamp examination of eyelids, conjunctiva, |
| | cornea, lens, iris, and anterior chamber. |
| Indirect | Evaluation of posterior segment abnormalities |
| Ophthalmoscopy | of the vitreous, optic nerve, peripheral retina, |
| (Dilated) | and retinal vasculature. |
| Intraocular | Measurement of IOP conducted using a |
| Pressure | Goldmann applanation tonometer. |

In the event of significant intraocular inflammation (IOI), low IOP, and/or vision loss, gonioscopy is performed as part of the full ophthalmic examination if feasible.

### Post-Injection Assessment

The post-injection assessment is performed on the study eye 0-5 minutes and 30±15 minutes following IVT aflibercept injection (Screening and/or supplemental), prophylactic IVT Ozurdex® implant injection, and/or IVT AAV2.7m8-aflibercept injection. The post-injection assessment involves an assessment of vision (e.g., count fingers) and measurement of IOP. Direct visualization to assess the central retinal artery, presence of retinal detachment, or presence of new intraocular hemorrhage may be performed.

### Fundus Autofluorescence

Fundus Autofluorescence (FAF) imaging is performed to assess occurrence of geographic atrophy.

Additional assessments include Color Fundus Photography (CFP), Optical coherence tomography (OCT) images of the optic nerve head and retinal nerve fiber layer, and Fluorescein Angiography (FA).

### Additional Imaging for Safety Management

Additional ocular examination and imaging assessments are captured in the presence of significant intraocular inflammation, as described below.
Slit Lamp Photography (including retroillumination photography and red reflex photography for assessment of PSC cataract) is performed for all study participants and in the event of significant intraocular inflammation. If red reflex photography of the iris cannot be obtained with a slit lamp camera, it is acceptable to use fundus photographs.
Anterior Segment OCT images are assessed in the event of significant intraocular inflammation.
Ultra-wide Field Color Fundus Photography (UWFCFP) is performed in the event of significant intraocular inflammation. If UWFCFP is not available, standard CFP images are used for safety management.
Ultra-wide Field Fluorescein Angiography (UWFFA) is performed in the event of significant intraocular inflammation. If UWFFA is not available, standard FA images are used for safety management.
Gonioscopy is performed prior to dilation on both eyes in the event of significant intraocular inflammation. A comparison is made between the two eyes in terms of pigment deposition in the chamber angle as well as the visible depth of the chamber angle.

### Other Assessments and Procedures

The following additional assessments and procedures are performed during this study.

### Aqueous Humor Sampling

Aqueous humor (AH) samples are collected during the study. AH samples are only collected if considered safe for the participant (as determined by the Investigator) and if the participant's IOP at the time of sampling is >10 mmHg.

AH samples are collected to determine aflibercept levels, e.g., at Week 10, Week 26, and Week 50/EOS study visits. In addition, AH samples are obtained prior to administration of the first supplemental aflibercept injection so long as it occurs at Week 10 or later. Following the first supplemental aflibercept injection, regularly scheduled AH sampling occurs if the visit happens at least 12 weeks after the most recent aflibercept injection.

An AH sample is obtained upon the occurrence of a participant's first episode of significant intraocular inflammation so long as the participant's IOP at the time of sampling is >10 mmHg and the Investigator believes it safe to obtain an AH sample in the presence of IOI. This sample is for the assessment of cells/material in the anterior chamber. Furthermore, if significant IOI exists, further samples of AH for aflibercept levels are not obtained until the inflammation has resolved for at least 12 weeks.

### Antibodies and Immune Responses

Anti-drug antibodies (ADA) are assessed in participant serum samples. ADA assessments include total antibodies to AAV.7m8 and neutralizing antibodies to AAV.7m8.

Presence of total anti-AAV.7m8 antibodies (TAbs) are measured in serum (and aqueous humor, where possible) in a ligand binding assay. Presence of neutralizing anti-AAV.7m8 antibodies (NAbs) is measured in serum (and aqueous humor, where possible) in a cell-based assay. NAb titer is measured in serum at Screening for the purpose of assessing participant eligibility. Participants with a serum Nab titer 1:125 or higher are excluded from participation in the study.

Humoral response against aflibercept is measured in serum (and aqueous humor, where possible) in a ligand binding assay.

Cellular immunity against AAV.7m8 capsid and aflibercept protein is assessed in whole blood in an enzyme-linked immune absorbent spot (ELISpot) assay.

### Aflibercept Protein Expression

Presence of total aflibercept protein is measured in serum (and aqueous humor, where possible) in a ligand binding assay.

### Viral Shedding

Tears from both eyes and whole blood are collected from a subset of participants. Viral shedding samples are collected after AAV2.7m8-aflibercept has been injected, and continue to be collected until 3 consecutive samples (for both blood and tears of both eyes) have tested negative for viral shedding. Samples are analyzed by quantitative-Polymerase Chain Reaction (q-PCR) for evidence of viral shedding related to AAV2.7m8-aflibercept.

### Participant Preference Survey

A Participant Preference Survey (PPS) is provided to participants for completion at the Week 26 and Week 50/EOS/or ET visit. The PPS is designed to gather feedback on participants' experience with study treatment and includes items on willingness to receive study treatment in the fellow eye and ability to manage the assigned steroid regimen. If a participant cannot complete the survey due to vision-related or other reasons, it may be administered by a study site staff member.

### Vitreous Humor and Other Ocular Tissue Sampling

If vitrectomy is medically necessary, a vitreous humor sample may be obtained. Vitreous humor samples are analyzed primarily for aflibercept concentrations. The remaining samples may be analyzed for other biomarkers. If any other surgical procedures become medically necessary in the study and/or fellow eye during the course of the study, other ocular tissues may be provided, including aqueous or vitreous humor and lens, to be analyzed for other biomarkers.

### Statistics and Study Analysis Populations

The safety population includes all participants who have received AAV2.7m8-aflibercept, and are analyzed according to the dose received. AEs are coded using the MedDRA (Most Recent Version) classification to give a preferred term (PT) and system organ class (SOC) for each event.

The efficacy analysis endpoints are analyzed at Week 26 (primary timepoint) and Week 50. Efficacy analyses include all subjects with study treatment and at least one post-study assessment. Baseline values for endpoints refer to measurements taken at the Screening Visit in which aflibercept was administered.

The primary efficacy endpoint, change in BCVA in ETDRS letters from baseline at Week 50/EOS, is analyzed by mixed model repeated measures (MMRM).

### V. Concomitant Medications and Treatments

Concomitant medications are any prescription or over-the-counter drugs other than study protocol-specified procedural medications and pre- and post-IVT injection medications used by a participant 30 days before Screening/Baseline until the participant's EOS or ET visit.

At the discretion of the evaluating physician, participants may continue to receive all medications and standard treatments administered for other conditions except for treatments noted in the exclusion criteria described above and as noted in the prohibited medications below.

The following medications are prohibited during the study:
Any systemic anti-VEGF agent, including bevacizumab.
Systemic drugs known to cause macular edema (e.g., fingolimod, tamoxifen, chloroquine/hydroxychloroquine).
Any anti-VEGF agent in the study eye other than the study drug or aflibercept injection 2 mg, as defined in the study protocol.
IVT steroids in the study eye (e.g., Ozurdex®, Iluvien® or Triesence®), unless for the treatment of (or prevention of) an ocular adverse event.
Systemic immunosuppressive drugs (e.g., intravenous steroids, methotrexate, azathioprine, cyclosporin, adalimumab, infliximab, etanercept), unless prescribed by the Investigator for intraocular inflammation refractory to topical, oral, or intravitreal steroids (inhaled or non-ocular topical steroids an NSAIDs are allowed).
Use of any other investigational study treatment or device within 30 days prior to screening and during the study.
Medications contraindicated for concomitant use with oral prednisone.
COVID-19 vaccination within 6 weeks prior to or 6 weeks after the Randomization Visit (Day −7).

Individuals with visually significant cataracts are not randomized or enrolled in the study. However, if a cataract develops during the study, cataract surgery in the study eye may be performed if clinically indicated and is scheduled no sooner than 3 months after AAV2.7m8-aflibercept injection at Day 1, and/or no sooner than 7 days after the last injection of supplemental aflibercept, and/or no sooner than 3 months after the resolution of active intraocular inflammation. Participants may continue on the study after cataract surgery. Individuals who develop AMD in the fellow (non-study) eye may receive standard of care therapy.

### VI. Safety and Adverse Events

The reporting period for adverse events (AEs), serious adverse events (SAEs), and adverse events of special interest (AESIs) begins at the time a subject signs the informed consent form until a participant completes the study, discontinues prematurely, or withdraws consent. If a participant discontinues prematurely after receiving AAV2.7m8-aflibercept, AEs are recorded until discontinuation or 30 days after the last study visit, whichever is later. Between the time of informed consent and study treatment administration, only AEs (non-serious and serious) assessed as related to study procedures are reported. After treatment administration (Day 1), all clinically significant AEs are reported. Each participant is followed until a) the end of the AE reporting period at 30 days after the last study visit or b) when any ongoing AAV2.7m8-aflibercept-related AEs and/or SAEs have resolved or become stable. Any clinically significant safety assessment that is associated with nAMD is not reported as an AE or SAE, unless judged by the Investigator to be more severe than expected for the subject's condition.

Participants that reach Week 50 are invited to participate in a separate long-term extension study with continued safety monitoring. Participants that received AAV2.7m8-aflibercept are followed for at least 5 years from date of AAV2.7m8-aflibercept dosing or until they are lost to follow-up, whichever occurs first.

### Adverse Events, Serious Adverse Events, and Adverse Events of Special Interest

An AE is any untoward medical occurrence in an enrolled participant regardless of its causal relationship to AAV2.7m8-aflibercept, including but not limited to: abnormal laboratory findings or clinical tests, including those that worsen from baseline and are clinically significant in the medical and scientific judgment of the Investigator; any new disease or exacerbation of an existing condition; recurrence of a sporadic medical condition (e.g., fever) not present at Screening/Baseline; adverse events related to a study protocol-mandated intervention, including those that occur prior to study treatment (e.g., aflibercept injection).

An SAE is any untoward medical occurrence in an enrolled participant, regardless of its causal relationship to AAV2.7m8-aflibercept, that results in death, a life-threatening condition, inpatient hospitalization or prolongation of existing hospitalization, or persistent or significant disability or incapacity or is a congenital anomaly or birth defect. Important medical events that may not meet these criteria may be considered SAEs when, based on appropriate medical judgment, they may jeopardize the participant or may require medical or surgical intervention to prevent one of the outcomes listed in above. All malignant neoplasms are assessed as serious in the category of “medically significant” if other seriousness criteria are not met. Any suspected transmission of an infectious agent by a medicinal agent is also considered a serious adverse event. Development or progression of a cataract and/or cataract surgery is not considered reportable as an SAE, unless the cataract or cataract surgery results in permanent disability.

Adverse events of special interest (AESIs) include sight-threatening adverse events in the study eye. An adverse event is sight-threatening if it meets one or more of the following: it causes a decrease of ≥30 letters in BCVA using an ETDRS visual acuity chart compared with the prior visit and is not associated with worsening of cataract; it requires surgical or medical intervention (e.g., conventional surgery, vitrectomy) to prevent permanent loss of sight (e.g., IOP>30 mmHg or ≤5 mmHg); and/or it causes severe intraocular inflammation (e.g., endophthalmitis [presumed infectious], papillitis, choroiditis, retinitis, vasculitis, or grade 3+ or 4+ anterior chamber cell/flare, vitreous cells/haze, or vitritis).

The Medical Dictionary for Regulatory Activities (MedDRA) is used to code all AEs.

Events are considered related to AAV2.7m8-aflibercept if there is a reasonable possibility of a causal relationship rather than if a causal relationship cannot be ruled out. When assessing causality, Investigators assess biological balancing (i.e., the probability that the suspect drug caused the AE must be weighed against the probability that an alternative candidate caused it).

### Management of Intraocular Inflammation and Low Intraocular Pressure

Additional ocular examination and imaging assessments are captured in the presence of active intraocular inflammation and/or significant vision loss (greater than or equal to 15 letters loss from Baseline).

If any new, worsening, or recurrent intraocular inflammation (with or without low intraocular pressure) is observed, additional assessments are collected to fully characterize inflammation location and severity. In all cases of new intraocular inflammation (with or without low intraocular pressure) BCVA and a full ophthalmic examination including slit lamp biomicroscopy, indirect ophthalmic exam, and IOP, as well as SD-OCT, are performed to assess the intraocular inflammation and/or pressure. The iris is carefully examined for the presence of transillumination defects, and slit lamp photos are obtained if present. In the absence of contraindications, intravitreal or sub-tenon corticosteroid, e.g., Triesence®, is considered as second-line steroid management for persistent inflammation or new cases of low IOP despite intensive treatment with topical difluprednate. Immunomodulating therapy is considered for persistent low IOP in the presence or absence of inflammation.

Cases of significant intraocular inflammation include one or more of:
Anterior segment inflammation characterized by an increase of 2 or more units as defined by the SUN criteria.
New keratic precipitates.
New or worsening of vitreous inflammation characterized by a 2-step increase in vitreous cells or vitreous haze.
Intraocular pressure≤5 mmHg or a ≥10 mmHg decrease from Baseline (new).
New iris transillumination defect.
Iris color change.
Cases of inflammation associated with a ≥10 letter loss in BCVA.

Additional assessments in these circumstances may include ad-hoc image collection for safety management of the study eye, including:
Gonioscopy of both eyes.
Anterior Segment OCT.
Ultra-wide Field Color Fundus Photos.
Ultra-wide Field Fluorescein Angiography.
Slit Lamp Photos.
Spectral Domain OCT.

If considered safe to collect, ad-hoc aqueous humor samples are collected for safety management at the first instance of significant intraocular inflammation for a participant. If intraocular inflammation is noted with IOP≤10 mmHg, no aqueous humor sample is taken.

1. A method for treating an ocular neovascular disease in an individual, the method comprising administering a unit dose of about 2×10¹¹ vector genomes (vg) or less of recombinant adeno-associated virus (rAAV) particles to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

2. The method of claim 1, wherein retinal fluid in the eye of the individual with an ocular neovascular disease is reduced.

3. A method for reducing retinal fluid in an eye of an individual with an ocular neovascular disease, the method comprising administering a unit dose of rAAV particles to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

4. The method of claim 3, wherein the individual has received at least one treatment of an anti-VEGF agent in about 12 weeks prior to administration of the unit dose of rAAV particles.

5. The method of claim 3 or claim 4, wherein the amount or presence of retinal fluid in the one eye of the individual is refractory to prior treatment with an anti-VEGF agent.

6. The method of claim 4 or claim 5, wherein the anti-VEGF agent is aflibercept.

7. The method of any one of claims 2-6, wherein the retinal fluid in the one eye is reduced by at least about 60%.

8. The method of any one of claims 2-7, wherein the retinal fluid in the one eye is reduced by about 80% compared to the level of retinal fluid in the one eye of the individual prior to administration of the rAAV to the individual.

9. The method of any one of claims 2-8, wherein the retinal fluid is subretinal fluid (SRF) or intraretinal fluid (IRF).

10. The method of any one of claims 3-9, wherein the unit dose of rAAV particles is about 6×10¹¹ vector genomes per eye (vg/eye) or less.

11. A method for treating an ocular neovascular disease in an individual, the method comprising:
(a) administering an anti-VEGF agent to one eye of the individual; and
(b) administering a unit dose of about 6×10¹¹ vector genomes (vg) or less of recombinant adeno-associated virus (rAAV) particles to the one eye of the individual after administration of the anti-VEGF agent, wherein the individual is a human, and wherein the rAAV particles comprise:
(i) a nucleic acid encoding a polypeptide comprising an amino acid sequence with at least about 95% identity to the amino acid sequence of SEQ ID NO: 35 and flanked by AAV2 inverted terminal repeats (ITRs), and
(ii) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

12. The method of claim 11, comprising administering the unit dose of rAAV particles to the one eye of the individual about 1 week or about 7 days after administration of the anti-VEGF agent.

13. The method of claim 11 or claim 12, comprising administering the anti-VEGF agent to the one eye of the individual on Day 1, and administering the unit dose of rAAV particles to the one eye of the individual on Day 8.

14. The method of any one of claims 11-13, wherein the anti-VEGF agent comprises aflibercept.

15. The method of claim 14, where the aflibercept is administered at a dose of about 2 mg by intravitreal injection.

16. The method of any one of claims 1-15, wherein the unit dose of rAAV particles is between about 6×10¹⁰ to about 6×10¹¹ vector genomes per eye (vg/eye).

17. The method of any one of claims 1-16, wherein the unit dose of rAAV particles is between about 6×10¹⁰ to about 2× 10¹¹ vector genomes per eye (vg/eye).

18. The method of any one of claims 1-16, wherein the unit dose of rAAV particles is between about 2×10¹¹ to about 6×10¹¹ vector genomes per eye (vg/eye).

19. The method of claim 18, wherein the unit dose of rAAV particles is about 2×10¹¹ or about 6×10¹¹ vector genomes per eye (vg/eye).

20. The method claim 19, wherein the unit dose of rAAV particles is about 2×10¹¹ vector genomes per eye (vg/eye).

21. The method claim 19, wherein the unit dose of rAAV particles is about 6×10¹¹ vector genomes per eye (vg/eye).

22. The method of any one of claims 1-21, wherein the individual has one or more symptoms of an ocular neovascular disease in the contralateral eye.

23. The method of any one of claims 1-22, further comprising administering a unit dose of rAAV particles to the contralateral eye of the individual.

24. The method of claim 23, wherein the administering the unit dose of rAAV particles to the contralateral eye is up to about 2 weeks after administering the unit dose of rAAV particles to the one eye.

25. The method of claim 24, wherein:
(a) the administering the unit dose of rAAV particles to the contralateral eye is on the same day as the administering the unit dose of rAAV particles to the one eye; or
(b) the administering the unit dose of rAAV particles to the contralateral eye is between about 1 day to about 14 days after administering the unit dose of rAAV particles to the one eye.

26. The method of claim 24 or claim 25, wherein the unit dose of rAAV particles administered to the contralateral eye of the individual comprises the same or less vector genomes per eye (vg/eye) than the unit dose of rAAV particles administered to the one eye of the individual.

27. The method of claim 23, wherein the administering the unit dose of rAAV particles to the contralateral eye is at least about 2 weeks after administering the unit dose of rAAV particles to the one eye.

28. The method of claim 27, wherein the unit dose of rAAV particles administered to the contralateral eye of the individual comprises more vector genomes per eye (vg/eye) than the unit dose of rAAV particles administered to the one eye of the individual.

29. The method of any one of claims 1-28, wherein the nucleic acid comprises the nucleic acid sequence of SEQ ID NO: 40 or a sequence having at least 85% identity thereto.

30. The method of any one of claims 1-29, wherein the polypeptide comprises the amino acid sequence of SEQ ID NO: 35.

31. The method of any one of claims 1-30, wherein the polypeptide comprises the amino acid sequence of SEQ ID NO: 41.

32. The method of any one of claims 1-31, wherein the polypeptide is aflibercept.

33. The method of any one of claims 1-32, wherein the nucleic acid further comprises a first enhancer region, a promoter region, a 5′UTR region, a second enhancer region, and a polyadenylation site.

34. The method of any one of claims 1-33, wherein the nucleic acid comprises, in the 5′ to 3′ order:
(a) a first enhancer region;
(b) a promoter region;
(c) a 5′UTR region;
(d) a nucleic acid encoding a polypeptide comprising an amino acid sequence with at least about 95% identity to the amino acid sequence of SEQ ID NO: 35;
(e) a second enhancer region; and
(f) a polyadenylation site;
and flanked by AAV2 inverted terminal repeats (ITRs).

35. The method of claim 33 or claim 34, wherein the first enhancer region comprises a CMV sequence comprising the sequence of SEQ ID NO: 22 or a sequence having at least 85% identity thereto.

36. The method of any one of claims 33-35, wherein the promoter region comprises a CMV sequence comprising the sequence of SEQ ID NO: 23 or a sequence having at least 85% identity thereto.

37. The method of any one of claims 34-36, wherein the nucleic acid encoding a polypeptide comprises the nucleic acid sequence of SEQ ID NO: 40 or a sequence having at least 85% identity thereto.

38. The method of any one of claims 34-37, wherein the polypeptide comprises the amino acid sequence of SEQ ID NO: 35 or a sequence having at least 95% identity thereto.

39. The method of any one of claims 34-38, wherein the polypeptide comprises the amino acid sequence of SEQ ID NO: 41 or a sequence having at least 95% identity thereto.

40. The method of any one of claims 34-39, wherein the polypeptide is aflibercept.

41. The method of any one of claims 33-40, wherein the 5′UTR region comprises, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto.

42. The method of any one of claims 33-41, wherein the second enhancer region comprises a full EES sequence comprising the sequence of SEQ ID NO: 26 or a sequence having at least 85% identity thereto.

43. The method of any one of claims 33-42, wherein the polyadenylation site comprises a HGH polyadenylation site comprising the sequence of SEQ ID NO: 27 or a sequence having at least 85% identity thereto.

44. The method of any one of claims 1-32, wherein the nucleic acid further comprises (a) a first enhancer region comprising a CMV sequence comprising the sequence of SEQ ID NO: 22 or a sequence having at least 85% identity thereto; (b) a promoter region, comprising a CMV sequence comprising the sequence of SEQ ID NO: 23 or a sequence having at least 85% identity thereto; (c) a 5′UTR region comprising, in 5′ to 3′ order, a TPL sequence comprising the sequence of SEQ ID NO: 24 or a sequence having at least 85% identity thereto, and an eMLP sequence comprising the sequence of SEQ ID NO: 25 or a sequence having at least 85% identity thereto; (d) a second enhancer region comprising a full EES sequence comprising the sequence of SEQ ID NO: 26 or a sequence having at least 85% identity thereto; and (e) a HGH polyadenylation site comprising the sequence of SEQ ID NO: 27 or a sequence having at least 85% identity thereto.

45. The method of any one of claims 1-44, wherein the nucleic acid comprises the sequence of SEQ ID NO: 39 or a sequence having at least 85% identity thereto.

46. The method of any one of claims 1-45, wherein the AAV2 capsid protein comprises the amino acid sequence LALGETTRPA (SEQ ID NO: 1) inserted between positions 587 and 588 of the capsid protein, wherein the amino acid residue numbering corresponds to an AAV2 VP1 capsid protein.

47. The method of any one of claims 1-46, wherein the AAV2 capsid protein comprises the amino acid sequence LGETTRP (SEQ ID NO: 14) inserted between positions 587 and 588 of the AAV2 VP1 comprising the sequence of SEQ ID NO: 13.

48. The method of any one of claims 1-47, wherein the AAV2 capsid protein comprises the amino acid sequence LALGETTRPA (SEQ ID NO: 1) inserted between positions 587 and 588 of the AAV2 VP1 comprising the sequence of SEQ ID NO: 13.

49. The method of any one of claims 1-46, wherein the rAAV particles comprise an AAV2 VP1 capsid protein comprising a GH loop that comprises the amino acid sequence of SEQ ID NO: 38 or an amino acid sequence having at least 90% sequence identity to SEQ ID NO: 38.

50. The method of any one of claims 1-49, wherein the administration of the unit dose of rAAV particles to the one eye and/or the contralateral eye is by intravitreal administration.

51. The method of any one of claims 1-50, wherein the unit dose of rAAV particles is in a pharmaceutical formulation.

52. The method of claim 51, wherein the pharmaceutical formulation comprises the rAAV particles, sodium chloride, sodium phosphate and a surfactant.

53. The method of claim 52, wherein the pharmaceutical formulation comprises about 150 to about 200 mM sodium chloride, about 1 to about 10 mM monobasic sodium phosphate, about 1 to about 10 mM dibasic sodium phosphate, about 0.0005% (w/v) to about 0.005% (w/v) poloxamer 188, and about 6×10¹³ to about 6×10¹⁰ vector genomes (vg) per mL (vg/mL) of the rAAV particles, wherein the pharmaceutical formulation has a pH of about 7.0 to about 7.5.

54. The method of claim 53, wherein the pharmaceutical formulation comprises about 180 mM sodium chloride, about 5 mM monobasic sodium phosphate, about 5 mM dibasic sodium phosphate, about 6×10¹² vg/mL of the rAAV particles, and about 0.001% (w/v) poloxamer 188, wherein the pharmaceutical formulation has a pH of about 7.3.

55. The method of claim 53, wherein the pharmaceutical formulation comprises about 180 mM sodium chloride, about 5 mM monobasic sodium phosphate, about 5 mM dibasic sodium phosphate, about 2×10¹² vg/mL of the rAAV particles, and about 0.001% (w/v) poloxamer 188, wherein the pharmaceutical formulation has a pH of about 7.3.

56. The method of claim 53, wherein the pharmaceutical formulation comprises about 180 mM sodium chloride, about 5 mM monobasic sodium phosphate, about 5 mM dibasic sodium phosphate, about 6×10¹¹ vg/mL of the rAAV particles, and about 0.001% (w/v) poloxamer 188, wherein the pharmaceutical formulation has a pH of about 7.3.

57. The method of any one of claims 1-56, wherein the unit dose of rAAV particles administered to the one eye and/or to the contralateral eye in a volume of about 25 μL to about 250 μL.

58. The method of claim 57, wherein the unit dose of rAAV particles administered to the one eye and/or to the contralateral eye comprises a volume of about 100 μL.

59. The method of claim 57, wherein the unit dose of rAAV particles administered to the one eye and/or to the contralateral eye comprises a volume of about 30 μL.

60. The method of any one of claims 1-59, wherein the individual received prior treatment for the ocular neovascular disease with an anti-VEGF agent.

61. The method of claim 60, wherein the individual has received 1 or 2 injections of an anti-VEGF agent in the one eye and/or in the contralateral eye prior to administration of the rAAV particles in the one eye and/or in the contralateral eye.

62. The method of any one of claim 1-3 or 7-59, wherein the individual has not received prior treatment for the ocular neovascular disease with an anti-VEGF agent.

63. The method of any one of claims 60-62, wherein the anti-VEGF agent is aflibercept.

64. The method of any one of claims 1-63, wherein the ocular neovascular disease is wet age-related macular degeneration (AMD), retinal neovascularization, choroidal neovascularization diabetic retinopathy, proliferative diabetic retinopathy, retinal vein occlusion, central retinal vein occlusion, branched retinal vein occlusion, diabetic macular edema, diabetic retinal ischemia, ischemic retinopathy, diabetic retinal edema, or any combination thereof.

65. The method of any one of claim 1-10 or 16-64, wherein the unit dose of rAAV particles is administered in combination with steroid treatment.

66. The method of claim 65, wherein the steroid treatment is a corticosteroid treatment.

67. The method of claim 65 or claim 66, wherein the steroid treatment is a systemic steroid treatment.

68. The method of any one of claims 65-67, wherein the steroid treatment is an oral steroid treatment.

69. The method of any one of claims 65-68, wherein the steroid treatment is a prednisone treatment.

70. The method of claim 65 or claim 66, wherein the steroid treatment is a topical steroid treatment.

71. The method of claim 70, wherein the steroid treatment is a difluprednate treatment.

72. The method of any one of claims 65-71, wherein the steroid is administered before, during and/or after administration of the unit dose of rAAV particles to the one eye and/or to the contralateral eye.

73. The method of any one of claims 70-72, wherein the steroid treatment is a topical steroid treatment and the topical steroid treatment is a daily steroid treatment for up to about 4 weeks, up to about 6 weeks, or up to about 8 weeks from administering the unit dose of rAAV particles.

74. The method of claim 73, wherein the topical steroid treatment comprises about four administrations of topical steroid per day on about week 1, about three administrations of topical steroid per day on about week 2, about two administrations of topical steroid per day on about week 3, and about one administration per day of topical steroid on about week 4; timing starting with and following administration of the unit dose of rAAV particles.

75. The method of claim 73, wherein the topical steroid treatment comprises about four administrations of topical steroid per day for about 3 weeks after administration of the unit dose of rAAV particles, followed by about 3 administrations of topical steroid per day for about 1 week, followed by about 2 administrations of topical steroid per day for about 1 week, and followed by about 1 administration of topical steroid per day for about 1 week.

76. The method of claim 73, wherein the topical steroid treatment comprises about four administrations of topical steroid per day for about four weeks, followed by about three administrations of topical steroid per day for about one week, followed by about two administrations of topical steroid per day for about one week, and followed by about one administration of topical steroid per day for about one week; timing starting at about one week prior to administration of the unit dose of rAAV particles.

77. The method of any one of claims 11-64, further comprising administering a topical steroid treatment.

78. The method of claim 77, wherein the topical steroid treatment is a difluprednate treatment.

79. The method claim 77 or claim 78, wherein the topical steroid treatment comprises about four administrations of topical steroid per day for about four weeks, followed by about three administrations of topical steroid per day for about one week, followed by about two administrations of topical steroid per day for about one week, and followed by about one administration of topical steroid per day for about one week; timing starting with and following administration of the anti-VEGF agent.

80. The method of any one of claims 71-79, wherein the topical steroid comprises difluprednate 0.05% at a dose of about 1 μg to about 3 μg.

81. The method of any one of claims 71-80, wherein the topical steroid comprises difluprednate 0.05% at a dose of about 2.5 μg.

82. The method of any one of claims 1-81, wherein the administering the unit dose of rAAV particles to the one eye and/or to the contralateral eye of the individual results in maintenance or a decrease of retinal thickness compared to the retinal thickness prior to administration of the unit dose of rAAV particles.

83. The method of claim 82, wherein the administering the unit dose of rAAV particles to the one eye and/or to the contralateral eye of the individual results in a decrease in retinal thickness compared to the retinal thickness prior to administration of the unit dose of rAAV particles.

84. The method of claim 83, wherein the decrease in retinal thickness is at least about 10% compared to the retinal thickness prior to administration of the unit dose of rAAV particles.

85. The method of any one of claims 82-84, wherein retinal thickness is central subfield thickness (CST) or central retinal thickness (CRT).

86. The method of any one of claims 1-85, wherein the administering the unit dose of rAAV particles to the one eye and/or to the contralateral eye of the individual results in maintenance or a decrease in macular volume compared to the macular volume prior to administration of the unit dose of rAAV particles.

87. The method of claim 86, wherein the administering the unit dose of rAAV particles to the one eye and/or to the contralateral eye of the individual results in a decrease in macular volume compared to the macular volume prior to administration of the unit dose of rAAV particles.

88. The method of claim 87, wherein the decrease in macular volume is at least about 10% compared to the macular volume prior to administration of the unit dose of rAAV particles.

89. The method of any one of claims 1-88, wherein the administering the unit dose of rAAV particles to the one eye and/or to the contralateral eye of the individual results in maintenance or an improvement of visual acuity compared to the visual acuity prior to administration of the unit dose of rAAV particles.

90. The method of claim 89, wherein the administering the unit dose of rAAV particles to the one eye and/or to the contralateral eye of the individual results in an improvement of visual acuity compared to the visual acuity prior to administration of the unit dose of rAAV particles.

91. The method of claim 89 or claim 90, wherein visual acuity is best corrected visual acuity (BCVA).

92. The method of any one of claims 1-91, wherein administration of the unit dose of rAAV particles to the one eye and/or to the contralateral eye of a plurality of individuals results in at least about 50% of the individuals in the plurality not requiring an anti-VEGF rescue treatment.

93. The method of claim 92, wherein at least about 50% of the individuals in the plurality do not require an anti-VEGF rescue treatment for at least about 20 weeks, at least about 36 weeks, at least about 52 weeks, at least about 56 weeks, or more after administration of the unit dose of rAAV particles.

94. The method of any one of claims 1-91, wherein administration of the unit dose of rAAV particles to the one eye and/or to the contralateral eye of a plurality of individuals results in at least about 67% of the individuals in the plurality not requiring an anti-VEGF rescue treatment.

95. The method of claim 94, wherein at least about 67% of the individuals in the plurality do not require an anti-VEGF rescue treatment for at least about 20 weeks, at least about 36 weeks, at least about 52 weeks, at least about 60 weeks, at least about 64 weeks, or at least about 66 weeks after administration of the unit dose of rAAV particles.

96. The method of any one of claims 1-91, wherein administration of the unit dose of rAAV particles to the one eye and/or to the contralateral eye of a plurality of individuals results in at least about 78% of the individuals in the plurality not requiring an anti-VEGF rescue treatment.

97. The method of claim 96, wherein at least about 78% of the individuals in the plurality do not require an anti-VEGF rescue treatment for at least about 20 weeks, at least about 36 weeks, or more after administration of the unit dose of rAAV particles.

98. The method of any one of claims 1-91, wherein administration of the unit dose of rAAV particles to the one eye and/or to the contralateral eye of a plurality of individuals results in 100% of the individuals in the plurality not requiring an anti-VEGF rescue treatment.

99. The method of claim 98, wherein 100% of the individuals in the plurality do not require an anti-VEGF rescue treatment for at least about 64 weeks, at least about 68 weeks, at least about 72 weeks, at least about 76 weeks, at least about 80 weeks, at least about 84 weeks, or more after administration of the unit dose of rAAV particles.

100. The method of any one of claims 1-99, wherein administration of the unit dose of rAAV particles to the one eye and/or to the contralateral eye of a plurality of individuals results in a reduction in the annualized anti-VEGF injection rate of at least about 80%, at least about 85%, at least about 87%, at least about 90%, at least about 95%, at least about 99%, or 100% compared to the annualized anti-VEGF injection rate prior to administration of the unit dose of rAAV particles.

101. The method of any one of claim 1-10, 16-76, or 80-100, wherein the ocular neovascular disease is wet age-related macular degeneration (AMD).

102. The method of any one of claim 1-3, 11-64, 77-83, 85-87, or 89-91, wherein the ocular neovascular disease is diabetic macular edema (DME).

103. The method of claim 102, wherein the administering the unit dose of rAAV particles to the one eye and/or to the contralateral eye of the individual results in a 2-step or in a 3-step improvement in Diabetic Retinopathy Severity Scale (DRSS).

104. A unit dose of about 6×10¹¹ vector genomes (vg) or less of recombinant adeno-associated virus (rAAV) particles for use in a method for treating an ocular neovascular disease in an individual, the method comprising administering said unit dose to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

105. A unit dose of rAAV particles for use in a method for reducing retinal fluid in an eye of an individual with an ocular neovascular disease, the method comprising administering said unit dose to one eye of the individual, wherein the individual is a human, and wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein.

106. The unit dose for use according to claim 104 or claim 105 wherein said method further comprises administering an anti-VEGF agent to said one eye of the individual before administration of said unit dose.

107. A method for treating an ocular neovascular disease in an individual, the method comprising administering of recombinant adeno-associated virus (rAAV) particles to one eye of the individual, wherein the individual is a human,
wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein, and
wherein the unit dose of rAAV particles is administered in combination with an oral steroid treatment and a topical steroid treatment or an oral steroid treatment and an intravitreal (IVT) steroid treatment.

108. The method of claim 107, wherein the oral steroid treatment and the topical steroid treatment or the oral steroid treatment and the IVT are a corticosteroid treatments.

109. The method of claim 107 or 108, wherein the topical steroid treatment is a difluprednate treatment.

110. The method of any one of claims 107-109, wherein the topical steroid treatment comprises difluprednate 0.05% at a dose of about 1 μg to about 3 μg.

111. The method of any one of claims 107-110, wherein the topical steroid treatment comprises difluprednate 0.05% at a dose of about 2.5 μg.

112. The method of any one of claims 107-111, wherein the topical steroid treatment comprises
a. about four administrations of topical steroid per day on about day 7 prior to administration of the unit dose of rAAV particles to about day 35 after administration of the unit does of rAAV particles,
b. about three administrations of topical steroid per day on about day 36 to about day 63 after administration of the unit dose of rAAV particles,
c. about two administrations of topical steroid per day on about day 64 to about day 91 after administration of the unit dose of rAAV particles,
d. about one administrations of topical steroid per day on about day 92 to about day 119 after administration of the unit dose of rAAV particles, and
e. about one administrations of topical steroid every other day starting on about day 120 to about day 147 after administration of the unit dose of rAAV particles.

113. The method of any one of claims 107-112, wherein the topical steroid treatment comprises
a. about four administrations of topical steroid per day about one week prior to administration of the unit dose of rAAV particles to about five weeks after administration of the unit does of rAAV particles,
b. about three administrations of topical steroid per day about 6 weeks to about 9 weeks after administration of the unit dose of rAAV particles,
c. about two administrations of topical steroid per day about 10 weeks to about 13 weeks after administration of the unit dose of rAAV particles,
d. about one administrations of topical steroid per day about 14 weeks to about 17 weeks after administration of the unit dose of rAAV particles, and
e. about one administrations of topical steroid every other day about 18 to about 21 weeks after administration of the unit dose of rAAV particles.

114. The method of any one of claims 107-113, wherein the oral steroid treatment is a prednisone treatment.

115. The method of claim 114, wherein the oral steroid treatment is administered at a dose of:
a. 1ess than about 0.8 mg/kg/day to about 1.00 mg/kg/day for the week prior to through week two following administering the unit dose of rAAV particles,
b. less than about 0.59 mg/kg/day to about 0.75 mg/kg/day on weeks three to five following administering the unit dose of rAAV particles,
c. less than about 0.4 mg/kg/day to about 0.6 mg/kg/day on week six after administering the unit dose of rAAV particles,
d. less than about 0.25 mg/kg/day to about 0.375 mg/kg/day on week seven after administering the unit dose of rAAV particles,
e. less than about 0.14 mg/kg/day to about 0.28 mg/kg/day on week eight after administering the unit dose of rAAV particles, and
f. less than about 0.063 mg/kg/day to about 0.125 mg/kg/day on week nine after administering the unit dose of rAAV particles.

116. The method of claim 114 or 115, wherein the oral steroid treatment is administered at a dose of:
a. 1ess than or equal to about 1.00 mg/kg/day for the week prior to through week two following administering the unit dose of rAAV particles,
b. less than or equal to about 0.75 mg/kg/day on weeks three to five following administering the unit dose of rAAV particles,
c. less than or equal to about 0.6 mg/kg/day on week six after administering the unit dose of rAAV particles,
d. less than or equal to about 0.375 mg/kg/day on week seven after administering the unit dose of rAAV particles,
e. less than or equal to about 0.28 mg/kg/day on week eight after administering the unit dose of rAAV particles, and
f. less than or equal to about 0.125 mg/kg/day on week nine after administering the unit dose of rAAV particles.

117. The method of any one of claims 114-116, where the oral steroid treatment is administered at a dose of about 0.125 mg/kg/day to about 0.14 mg/kg/day on the first four days of week nine after administering the unit dose of rAAV particles and about 0.0625 mg/kg/day to about 0.070 mg/kg/day on the first four days of week nine after administering the unit dose of rAAV particles if the individual weighs 70 kg or more.

118. The method of any one of claims 107-117, wherein the IVT steroid treatment is a triamcinolone acetonide treatment or a dexamethasone treatment.

119. The method of claim 118, wherein the IVT steroid treatment is a triamcinolone acetonide treatment of about 2 mg.

120. The method of claim 118 or 119, wherein the IVT steroid treatment comprises administration of the IVT steroid treatment one week prior to administration of the unit dose of rAAV particles and at week 12 after administration of the unit dose of rAAV particles.

121. The method of claim 118, wherein the IVT steroid treatment is a dexamethasone of about 0.7 mg.

122. The method of claim 121, wherein the IVT steroid treatment is an intravitreal implant one week prior to administration of the unit does of rAAV particles.

123. The method of any one of claims 107-122, wherein the oral steroid treatment and/or the topical steroid treatment is administered before, during and/or after administration of the unit dose of rAAV particles.

124. A unit dose of recombinant adeno-associated virus (rAAV) particles for use in a method for treating an ocular neovascular disease in an individual, the method comprising administering said unit dose to one eye of the individual, wherein the individual is a human,
wherein the rAAV particles comprise:
a) a nucleic acid encoding a polypeptide comprising an amino acid sequence with at least about 95% identity to the amino acid sequence of SEQ ID NO: 35 and flanked by AAV2 inverted terminal repeats (ITRs), and
b) an AAV2 capsid protein comprising an amino acid sequence LGETTRP (SEQ ID NO: 14) inserted between positions 587 and 588 of the capsid protein, wherein the amino acid residue numbering corresponds to an AAV2 VP1 capsid protein, and
wherein the unit dose of rAAV particles is administered in combination with an oral steroid treatment and a topical steroid treatment or an oral steroid treatment and an intravitreal (IVT) steroid treatment.
